(19) United States
(12) Patent Application Publication
(10) Pub. No.: US 2014/0309229 A1
(43) Pub. Date: Oct. 16, 2014

(54) METHODS FOR SELECTING AND TREATING CANCER IN PATIENTS WITH IGF-1R/IR INHIBITORS

(71) Applicant: BRISTOL-MYERS SQUIBB COMPANY, Princeton, NJ (US)

(72) Inventors: Fei Huang, Princeton, NJ (US); Han Chang, Princeton Junction, NJ (US); Joan M. Carboni, Yardley, PA (US)

(73) Assignee: BRISTOL-MYERS SQUIBB COMPANY, Princeton, NJ (US)

(21) Appl. No.: 14/351,213

(22) PCT Filed: Oct. 12, 2012
(86) PCT No.: PCT/US2012/059982

§ 371 Date: Apr. 11, 2014

Related U.S. Application Data
(60) Provisional application No. 61/546,756, filed on Oct. 13, 2011.
(60) Provisional application No. 61/566,773, filed on Dec. 5, 2011.

(51) Int. Cl. C12Q 1/68; A61K 31/53

(52) U.S. Cl. CPC C12Q 1/6881; A61K 31/53
USPC 514/243; 435/6.11

(57) ABSTRACT

The present invention relates generally to the field of pharmacogenomics, and more specifically to methods and procedures to determine drug sensitivity in patients to allow the identification of individualized genetic profiles which will aid in selecting cancer patients that are responsive to IGF-1R/IR inhibition.

This application claims benefit to provisional application U.S. Ser. No. 61/546,756 filed Oct. 13, 2011; and to provisional application U.S. Ser. No. 61/566,773, filed Dec. 5, 2011; under 35 U.S.C. §119(e). The entire teachings of the referenced applications are incorporated herein by reference.

### FIELD OF THE INVENTION

The present invention relates generally to the field of pharmacogenomics, and more specifically to methods and procedures to determine drug sensitivity in patients to allow the identification of individualized genetic profiles which will aid in selecting cancer patients that are responsive to IGF-1R/IR inhibition.

### BRIEF DESCRIPTION OF THE SEQUENCE LISTING

Incorporated herein by reference in its entirety is a Sequence Listing entitled, “11826.PCT_ST25.txt”, comprising SEQ ID NO:1 through SEQ ID NO:11, which include nucleic acid and/or amino acid sequences disclosed herein. The Sequence Listing has been submitted herewith in IBM/PC MS-DOS text format via EFS. The Sequence Listing was first created on Oct. 12, 2012, and is 55 KB in size.

### BACKGROUND OF THE INVENTION

Targeted agents have emerged as important therapies in the treatment of a variety of human malignancies. Initial success is often hampered by a relatively rapid acquisition of drug resistance and subsequent relapse particularly in patients with advanced disease. Like conventional chemotherapy drugs, to which resistance has been well established as an important challenge in cancer therapy, the more recently developed kinase inhibitors are also subject to acquired resistance (Jänne et al., Nat. Rev. Drug Discov., 8(9):709-723 (2009); Engelman et al., Curr. Opin. Genet. Dev., 18:73-79 (2008)). The mechanisms of acquired drug resistance are beginning to be elucidated largely through two strategies: one is the molecular analysis of clinical specimens from patients who initially had clinical response to treatment therapy then relapsed on the drug; another is through in vitro cell culture modeling. The latter involves culturing drug-sensitive tumor-derived cell lines in the presence of continuous drug exposure until most of the cells are eliminated and then the cultures are eventually enriched with drug-resistant cell populations, which then can be characterized by genomic approaches to identify resistance mechanisms (Janne et al., Nat. Rev. Drug Discov., 8(9):709-723 (2009); Engelman et al., Curr. Opin. Genet. Dev., 18:73-79 (2008)).

Since activation and expression of insulin-like growth factor (IGF) signaling components contribute to proliferation, survival, angiogenesis, metastasis, and resistance to anti-cancer therapies in many human malignancies (7), the IGF system has become an attractive therapeutic target. The IGF system consists of two closely related receptors insulin receptor (IR), the type I-IGF receptor (IGF-1R/IR), and three ligands (IGF-I, IGF-II, and insulin). IR/IGF1R hybrid receptors signal similarly to IGF1R holoreceptors and have recently been implicated in cancer (Denley et al., Cytokine Growth Factor Rev., 16:421-439 (2005); Pandini et al., Clin. Cancer Res., 5:1935-1944 (1999)).

Insulin receptor plays an important role in regulating IGF action, either as a hybrid or holoreceptor, and IGF-1R/IR hybrid receptors are activated by IGF-I and IGF-II (Morrione et al., Proc. Natl. Acad. Sci. USA, 94:3777-3782 (1997)). The central components of the insulin-like growth factor (IGF) system consists of closely related receptors, the type I and II-IGF receptors (IGF-1R/IR and IGF-2R), two insulin receptor (IR) isoforms (IR-A and IR-B), three ligands (IGF-I, IGF-II, and insulin), and several IGF-binding proteins (IGFBP1-6). IGF-1R/IR hybrid receptors have recently been implicated in cancer and are activated by IGF-I and IGF-II with signals similar to the IGF-1R/IR homo-receptors (Morrione, A. et al., “Insulin-like growth factor II stimulates cell proliferation through the insulin receptor”, Proc. Natl. Acad. Sci. USA, 94:3777-3782 (1997); Denley, A. et al., “Molecular interactions of the IGF system”, Cytokine Growth Factor Rev., 16:421-439 (2005); and Pandini, G. et al., “Insulin and insulin-like growth factor-I (IGFI) receptor overexpression in breast cancers leads to insulin/IGF-I hybrid receptor overexpression: evidence for a second mechanism of IGF-I signaling”, Clin. Cancer Res., 5:1935-1944 (1999)). Binding of IGF ligands to the receptors results in autophosphorylation of receptors followed by phosphorylation of adaptor proteins IRS1, IRS2, and SHC, which are essential transducers and amplifiers of IGF signaling, triggers activation of mitogenic signaling pathways [Ras/Raf/mitogen-activated protein kinase (MAPK)] and antiapoptotic/survival pathways (PI3K-Akt/mTor) (LeRoith, D. et al., “The insulin-like growth factor system and cancer”, Cancer Lett., 195:127-137 (2003); and Baserga, R. et al., “The IGF-I receptor in cancer biology”, Int. J. Cancer, 107:873-877 (2003)).

Inhibition of both IGF-1R/IR and IR may be necessary to completely disrupt the malignant phenotype regulated by this signaling pathway (Law et al., Cancer Res., 68(24):10238-10246 (Dec. 15, 2008)). IGF signaling pathway is a major regulator of cellular proliferation, stress response, apoptosis, and transformation in mammalian cells, which is dysregulated and activated in a wide range of human cancers and this system is becoming one of the most intensively investigated molecular targets in oncology. Currently, there are close to 30 drug candidates being investigated that target the IGF-1R/IR receptors and a number of them are in clinical trials including IGF-1R/IR antibodies and small molecule inhibitors (Gualberto et al., Oncogene, 28(34):3009-3021 (2009); Rodon et al., Mol. Cancer Ther., 7(9):2575-2588 (2008); Weroha et al., J. Mamm. Gland Biol. Neoplasia, 13:471-483 (2008)).

BMS-754807 is a potent and selective reversible small molecule inhibitor of IGF1R family kinases, it targets both IGF-1R/IR and IR and has a wide spectrum of antitumor efficacy (Carboni et al., “BMS-754807, a small molecule inhibitor of IGF1R for clinical development”, Proceedings of the 100th Annual Meeting of the American Association for Cancer Research, Apr. 18-22, 2009, Denver, Colo., Abstract No. 1742). Targeting IGF-1R/IR signaling results in cancer cell growth inhibition both in vitro and in vivo by BMS-754807. This drug is currently in clinical development for the treatment of a variety of human cancers and pre-clinical defined efficacious exposures have been achieved with oral administration of single, tolerable doses in humans (Clements et al., AACR-NCI-EORTC Molecular Targets and Cancer Therapeutics Meeting 2009, Abstract No. A101) and pharmacological activity of BMS-754807 on pharmacodynamic biomarkers has been observed in cancer patients (Desai et al., AACR-NCI-EORTC Molecular Targets and Cancer Therapeutics Meeting 2009, Abstract No. A109).

In addition, early clinical evidence has demonstrated that anti-IGF-1R/IR antibodies have promising clinical benefit as a single agent or in combination with chemotherapy (Olmos et al., J. Clin. Oncol., 26:553s (2008); Tolcher et al., J. Clin. Oncol., 25:118s (2007); Karp et al., ASCO Meeting Abstracts 2008, 26 15_suppl:8015; Haluska et al. ASCO Meeting Abstracts 2007, 25 18_suppl:3586). With increasing numbers of small molecular IGF-1R/IR inhibitors entering clinical testing, it is highly probable they will soon provide definitive data on their value in future cancer treatments. However, like other cancer drugs, the IGF-1R/IR antibodies and small molecule inhibitors could also face a very important and general drawback, i.e., development of resistance.

New prognostic and predictive markers, which may facilitate individualized patient therapy are needed to accurately predict patient response to treatments, and in particular, identify the development of resistance to small molecule or biological molecule drugs, in order to identify the best treatment regimens. The problem may be solved by the identification of new parameters that could better predict the patient's sensitivity to treatment. The classification of patient samples is a crucial aspect of cancer diagnosis and treatment. The association of a patient's response to a treatment with molecular and genetic markers can open up new opportunities for treatment development in non-responding patients, or distinguish a treatment's indication among other treatment choices because of higher confidence in the efficacy. Further, the pre-selection of patients who are likely to respond well to a medicine, drug, or combination therapy may reduce the number of patients needed in a clinical study or accelerate the time needed to complete a clinical development program (Cockett, M. et al., Curr. Opin. Biotechnol., 11:602-609 (2000)).

However, the ability to determine which patients are responding to IGF-1R/IR therapies or predict drug sensitivity in patients is particularly challenging because drug responses reflect not only properties intrinsic to the target cells, but also a host's metabolic properties. Efforts to use genetic information to predict or monitor drug response have primarily focused on individual genes that have broad effects, such as the multidrug resistance genes mdr1 and mrp1 (Sonneveld, P., J. Intern. Med., 247:521-534 (2000)).

The development of microarray technologies for large scale characterization of gene mRNA expression pattern has made it possible to systematically search for molecular markers and to categorize cancers into distinct subgroups not evident by traditional histopathological methods (Khan, J. et al., Cancer Res., 58:5009-5013 (1998); Alizadeh, A. A. et al., Nature, 403:503-511 (2000); Bittner, M. et al., Nature, 406:536-540 (2000); Khan, J. et al., Nature Medicine, 7(6):673-679 (2001); and Golub, T. R. et al., Science, 286:531-537 (1999); Alon, U. et al., Proc. Natl. Acad. Sci. USA, 96:6745-6750 (1999)). Such technologies and molecular tools have made it possible to monitor the expression level of large numbers of transcripts within a cell population at any given time (see, e.g., Schena et al., Science, 270:467-470 (1995); Lockhart et al., Nature Biotechnology, 14:1675-1680 (1996); Blanchard et al., Nature Biotechnology, 14:1649 (1996); U.S. Pat. No. 5,569,588 to Ashby et al.).

Recent studies demonstrate that gene expression information generated by microarray analysis of human tumors can predict clinical outcome (van't Veer, L. J. et al., Nature, 415:530-536 (2002); Shipp, M. et al., Nature Medicine, 8(1):68-74 (2002); Glinsky, G. et al., J. Clin. Invest., 113(6):913-923 (2004)). These findings bring hope that cancer treatment will be vastly improved by better predicting and monitoring the response of individual tumors to therapy.

Needed are new and alternative methods and procedures to determine drug sensitivity or monitor response in patients to allow the development of individualized diagnostics which may be beneficial to treating diseases and disorders based on patient response at the molecular level, particularly cancer.

### SUMMARY OF THE INVENTION

The invention provides methods and procedures for determining patient sensitivity to one or more IGF-1R/IR agents, methods for treating patients with IGF-1R/1R agents, methods for designing personalized therapeutic regiments for patients with IGF-1R/1R agents either alone or in combination with other agents, in addition to diagnostic methods and kits thereof.

The present invention relates to the identification of several biomarkers, either alone or in combination, for use in identifying patient sensitivity and/or resistance to IGF-1R/IR inhibition. Specifically, the invention is directed to methods of identifying patients who may be susceptible to IGF-1R/IR inhibitor resistance, or who are resistant to IGF-1R/IR inhibition, comprising the step of measuring the copy number of IRS2 in a patient, wherein an elevated copy number of IRS2 relative to a control is indicative of sensitivity to IGF-1R/IR inhibition irrespective of said patient's KRAS mutant status.

The present invention relates to the identification of several biomarkers for use in identifying sensitivity and/or resistance to IGF-1R/IR inhibition. Specifically, the invention is directed to methods of identifying patients who may be responsive to IGF-1R/IR inhibition, or who are resistant to IGF-1R/IR inhibition, comprising the step of measuring the copy number of IRS2 in a patient, wherein a normal or non-amplified copy number of IRS2 (N=2) relative to a control is indicative of resistance, or a propensity to become resistant, to IGF-1R/IR inhibition if said patient harbors a KRAS mutation.

The present invention relates to the identification of several biomarkers for use in identifying sensitivity and/or resistance to IGF-1R/IR inhibition. Specifically, the invention is directed to methods of identifying patients who may be susceptible to IGF-1R/IR inhibitor resistance, or who are resistant to IGF-1R/IR inhibition, comprising the step of measuring the copy number of IRS2 in a patient, wherein a normal or non-amplified copy number of IRS2 relative to a control is indicative of decreased response or resistance, or a propensity to become resistant, to IGF-1R/IR inhibition if said patient harbors a KRAS mutation, particularly mutations in codons 12 and/or 13 of KRAS.

The invention is also directed to methods of identifying patients who may be susceptible to IGF-1R/IR inhibitor resistance, or who are resistant to IGF-1R/IR inhibition, comprising the step of measuring the expression level of IGFBP6 in a patient, wherein an elevated level of IGFBP6 relative to a control is indicative of decreased sensitivity or resistance, or a propensity to become resistant, to IGF-1R/IR inhibition.

The invention is also directed to methods of identifying patients who may be susceptible to IGF-1R/IR inhibitor resistance, or who are resistant to IGF-1R/IR inhibition, comprising the step of measuring the expression level of IGFBP6 in a patient, wherein a decreased or normal level of IGFBP6 relative to a control is indicative of sensitivity to IGF-1R/IR inhibition.

The invention is also directed to methods of identifying patients who may be susceptible to IGF-1R/IR inhibitor resistance, or who are resistant to IGF-1R/IR inhibition, comprising the step of measuring both the expression level of IGFBP6 in a patient in addition to assessing a patient's KRAS status, wherein a decreased or normal level of IGFBP6 relative to a control in a patient that is KRAS wild-type, is indicative of sensitivity to IGF-1R/IR inhibition.

The invention is also directed to methods of identifying patients who may be susceptible to IGF-1R/IR inhibitor resistance, or who are resistant to IGF-1R/IR inhibition, comprising the step of measuring both the expression level of IGFBP6 in a patient in addition to assessing a patient's KRAS status, wherein an elevated level of IGFBP6 relative to a control in a patient that is KRAS wild-type, is indicative of decreased sensitivity or resistance, or a propensity to become resistant, to IGF-1R/IR inhibition.

The invention is directed to methods of identifying patients who are sensitive to IGF-1R/IR inhibition, comprising the step of measuring the copy number of IRS2 in a patient in addition to assessing a patient's KRAS status, wherein a normal or non-amplified copy number of IRS2 relative to a control in conjunction with a patient harboring a KRAS mutation, is indicative of resistance, or a propensity to become resistant, to IGF-1R/IR inhibition.

The present invention relates to the identification of a gene mutation for use in identifying resistance to IGF-1R/IR inhibition. Specifically, the invention is directed to methods of identifying patients who may be susceptible to IGF-1R/IR inhibitor resistance, or who are resistant to IGF-1R/IR inhibition, comprising the step of measuring a KRAS mutation, particularly in codon G13, in a patient, wherein the G13D mutation is indicative of resistance to IGF-1R/IR inhibition.

The invention is directed to methods of identifying patients who are sensitive to IGF-1R/IR inhibition, comprising the step of measuring the copy number of IRS2 in a patient in addition to assessing a patient's KRAS status, wherein a normal or non-amplified copy number of IRS2 relative to a control in conjunction with a patient harboring a KRAS mutation, is indicative of resistance, or a propensity to become resistant, to IGF-1R/IR inhibition, wherein said mutant is in codon 12, 13, 61 and/or 146 of KRAS.

The invention is directed to methods of identifying patients who are sensitive to IGF-1R/IR inhibitor resistance, or who are resistant to IGF-1R/IR inhibition, comprising the step of measuring the copy number of IRS2 in a patient in addition to assessing a patient's KRAS status, wherein a normal or non-amplified copy number of IRS2 relative to a control in conjunction with a patient harboring a KRAS mutation, is indicative of resistance, or a propensity to become resistant, to IGF-1R/IR inhibition.

The invention is directed to methods of identifying patients who may be susceptible to IGF-1R/IR inhibitor resistance, or who are resistant to IGF-1R/IR inhibition, comprising the step of measuring the copy number of IRS2 in a patient in addition to assessing a patient's KRAS status, wherein a normal or non-amplified copy number of IRS2 relative to a control in conjunction with a patient harboring a KRAS mutation, is indicative of resistance, or a propensity to become resistant, to IGF-1R/IR inhibition, wherein said mutant is in codon 12 and/or 13 of KRAS.

The invention is directed to methods of identifying patients who may be susceptible to IGF-1R/IR inhibitor resistance, or who are resistant to IGF-1R/IR inhibition, comprising the step of measuring the copy number of IRS2 in a patient in addition to assessing a patient's KRAS status, wherein a normal or non-amplified copy number of IRS2 relative to a control in conjunction with a patient harboring a KRAS mutation, is indicative of resistance, or a propensity to become resistant, to IGF-1R/IR inhibition, wherein said mutant is a G12 or G13D KRAS mutant.

The invention is directed to methods of identifying patients who may be susceptible to IGF-1R/IR inhibitor resistance, or who are resistant to IGF-1R/IR inhibition, comprising the step of measuring the copy number of IRS2 in a patient in addition to assessing a patient's KRAS status, wherein a normal or non-amplified copy number of IRS2 relative to a control in conjunction with a patient harboring a KRAS mutation, is indicative of resistance, or a propensity to become resistant, to IGF-1R/IR inhibition, wherein said mutant is in codon 12, 13, 61 and/or 147 of KRAS.

The invention is directed to methods of identifying patients who may be susceptible to IGF-1R/IR inhibitor resistance, or who are resistant to IGF-1R/IR inhibition, comprising the step of measuring the copy number of IRS2 in a patient, assessing a patient's KRAS status, and measuring the expression level of IGFBP6, wherein a normal or non-amplified copy number of IRS2 relative to a control in conjunction with a patient having a wild-type KRAS in addition to a normal or decreased expression level of IGFBP6, is indicative of sensitivity to IGF-1R/IR inhibition.

The invention is also directed to methods of identifying patients who may be susceptible to IGF-1R inhibitor sensitivity to IGF-1R/IR inhibition, comprising the step of measuring the expression level of IGFBP6 in a patient, wherein an elevated level of IGFBP6 relative to a control is indicative of less responsive or resistance to IGF-1R/IR inhibition; whereas a decreased or normal level of IGFBP6 relative to a control is indicative of sensitivity to IGF-1R/IR inhibition, particular in a patient that is KRAS wild-type.

The invention is directed to methods of identifying patients who may be susceptible to IGF-1R/IR inhibitor resistance, or who are resistant to IGF-1R/IR inhibition, comprising the step of measuring the copy number of IRS2 in a patient, assessing a patient's KRAS status, and measuring the expression level of IGFBP6, wherein a normal or non-amplified copy number of IRS2 relative to a control in conjunction with a patient having a wild-type KRAS in addition to an elevated expression level of IGFBP6, is indicative of resistance, or a propensity to become resistant, to IGF-1R/IR inhibition.

The invention is directed to methods of identifying patients who may be susceptible to IGF-1R inhibitor sensitivity to IGF-1R/IR inhibition, comprising the step of measuring the copy number of IRS2 in a patient in addition to assessing a patient's KRAS status, wherein an elevated IRS2 copy number relative to normal or non-amplified copy number of IRS2 in conjunction with a patient harboring a KRAS mutation, is indicative of responsive or sensitive to IGF-1R/IR inhibition, wherein said mutant is in codon 12, 13, 61 and/or 146 of KRAS.

The present invention is directed to methods for predicting the likelihood a patient will respond therapeutically to a cancer treatment comprising the administration of an IGF-1R/IR inhibitor, comprising (a) measuring the copy number of IRS2 in a sample from said patient, and if said sample indicates said patent has an increased or elevated IRS2 copy number, (b) assessing the KRAS status of said patient, and (c) predicting an increased likelihood said patient will be sensitive to said cancer treatment if said patient has an increased or elevated IRS2 copy number in conjunction with the presence of a KRAS mutation other than a G13D mutation, or if said patient has an increased or elevated IRS2 copy number in conjunction with the presence of a wild type KRAS.

The present invention is directed to methods for predicting the likelihood a patient will respond therapeutically to a cancer treatment comprising the administration of an IGF-1R/IR inhibitor, comprising (a) measuring the copy number of IRS2 in a sample from said patient, and if said sample indicates said patent has an increased or elevated IRS2 copy number, (b) assessing the KRAS mutation status of said patient, (c) assessing the BRAF mutation status of said patient, and (d) predicting an increased likelihood said patient will be sensitive to said cancer treatment if said patient has an increased or elevated IRS2 copy number in conjunction with both the presence of a KRAS mutation other than a G13D mutation and wild type BRAF, or if said patient has an increased or elevated IRS2 copy number in conjunction with the presence of a wild type KRAS and wild type BRAF.

The present invention is directed to methods for predicting the likelihood a patient will respond therapeutically to a cancer treatment comprising the administration of an IGF-1R/IR inhibitor, comprising the steps of: (a) measuring the copy number of IRS2 in a sample from said patient, and if said sample indicates said patent has a normal or decreased IRS2 copy number, (b) assessing the KRAS status of said patient, and (c) predicting an increased likelihood said patient will be at least partially resistant to said cancer treatment if said patient has a normal or decreased IRS2 copy number in conjunction with the presence of a KRAS mutation.

The present invention is directed to methods for predicting the likelihood a patient will respond therapeutically to a cancer treatment comprising the administration of an IGF-1R/IR inhibitor, comprising: (a) measuring the copy number of IRS2 in a sample from said patient, and if said sample indicates said patent has a normal or decreased IRS2 copy number, (b) assessing the KRAS status of said patient, (c) if said patient is KRAS wild type, further comprising the steps of: (d) measuring the expression level of IGFBP6 in a sample from said patient, and (e) predicting an increased likelihood said patient will respond to said cancer treatment if said sample shows said patient has a normal or decreased expression level of IGFBP6, and predicting a decreased likelihood said patient will respond to said cancer treatment if said sample shows said patient has an elevated expression level of IGFBP6.

The present invention is directed to methods for predicting the likelihood a patient will respond therapeutically to a cancer treatment comprising the administration of an IGF-1R/IR inhibitor, comprising (a) measuring the expression level of IR-A in a sample from said patient, (b) assessing the KRAS mutation status of said patient, (c) assessing the BRAF mutation status of said patient, and (d) predicting an increased likelihood said patient will be sensitive to said cancer treatment if said patient has an increased or elevated IR-A expression level in conjunction with both the presence of a wild type KRAS and wild type BRAF.

The present invention is directed to methods for predicting the likelihood a patient will respond therapeutically to a cancer treatment comprising the administration of an IGF-1R/IR inhibitor, comprising (a) measuring the expression level of IGFBP6 in a sample from said patient, (b) assessing the KRAS mutation status of said patient, and (c) predicting an increased likelihood said patient will be sensitive to said cancer treatment if said patient has a decreased IGFPB6 expression level in conjunction with the presence of a wild type KRAS.

The present invention is directed to methods for predicting the likelihood a patient will respond therapeutically to a cancer treatment comprising the administration of an IGF-1R/IR inhibitor, comprising (a) measuring the expression level of IGFBP6 in a sample from said patient, (b) assessing the BRAF mutation status of said patient, and (c) predicting an increased likelihood said patient will be sensitive to said cancer treatment if said patient has a decreased IGFPB6 expression level in conjunction with the presence of wild type BRAF.

The present invention is directed to methods for predicting the likelihood a patient will respond therapeutically to a cancer treatment comprising the administration of an IGF-1R/IR inhibitor, comprising (a) measuring KRAS mutation status of said patient, and (b) predicting an decreased likelihood said patient will respond therapeutically to said cancer treatment if said patient has a G13D KRAS mutation.

The present invention is directed to methods for predicting the likelihood a patient will respond therapeutically to a cancer treatment comprising the administration of an IGF-1R/IR inhibitor, comprising (a) measuring BRAF mutation status of said patient, and (b) predicting an decreased likelihood said patient will respond therapeutically to said cancer treatment if said patient has a V600E BRAF mutation.

The present invention is directed to methods for predicting the likelihood a patient will respond therapeutically to a cancer treatment comprising the administration of an IGF-1R/IR inhibitor, comprising (a) measuring the expression level of IGF1R in a sample from said patient, and if said sample indicates said patent has an increased or elevated IGF1R expression level, (b) assessing the KRAS status of said patient, and (c) predicting an increased likelihood said patient will be sensitive to said cancer treatment if said patient has an increased or elevated IGF1R expression level in conjunction with the presence of a KRAS mutation other than a G13D mutation.

The present invention is directed to methods for predicting the likelihood a patient will respond therapeutically to a cancer treatment comprising the administration of an IGF-1R/IR inhibitor, comprising (a) measuring the expression level of IGFBP6 in a sample from said patient, (b) assessing the KRAS mutation status of said patient, (c) assessing the BRAF mutation status of said patient, and (d) predicting an increased likelihood said patient will be sensitive to said cancer treatment if said patient has a decreased IGFPB6 expression level in conjunction with both the presence of a wild type KRAS and wild type BRAF.

The present invention also provides a method of identifying a treatment regimen for a patient suffering from cancer comprising the step of: (i) measuring the copy number of IRS2 from a biological sample of said patient cell, wherein if elevated copy number of IRS2 is present, administering a therapeutically acceptable amount of an IGF-1R/IR inhibitor to said patient.

The present invention also provides a method of identifying a treatment regimen for a patient suffering from cancer comprising the step of: (i) measuring the copy number of IRS2 from a biological sample of said patient cell, wherein if elevated copy number of IRS2 is present, administering a therapeutically acceptable amount of an IGF-1R/IR inhibitor to said patient. Wherein said the cancer is a solid tumor, an advanced solid tumor, a metastatic solid tumor, a neoplasm, sarcoma, colon, and/or breast cancer, or other cancer outlined herein.

The present invention also provides a method of identifying a treatment regimen for a patient suffering from cancer comprising the step of: (i) measuring the copy number of IRS2 from a biological sample of said patient cell, and (ii) assessing the KRAS status of said patient, wherein if a normal or non-elevated copy number of IRS2 is present in conjunction with a KRAS mutation, administering an IGF-1R/IR inhibitor in combination with one or more IGF-1R/IR inhibitors and/or one or more other agents.

The present invention also provides a method of identifying a treatment regimen for a patient suffering from cancer comprising the step of: (i) measuring the copy number of IRS2 from a biological sample of said patient cell, and (ii) assessing the KRAS status of said patient, wherein if a normal or non-elevated copy number of IRS2 is present in conjunction with a KRAS mutation, administering an IGF-1R/IR inhibitor in combination with one or more IGF-1R/IR inhibitors and/or one or more other agents. Wherein said the cancer is a solid tumor, an advanced solid tumor, a metastatic solid tumor, a neoplasm, sarcoma, colon, and/or breast cancer, or other cancer outlined herein.

The present invention also provides a method of identifying a treatment regimen for a patient suffering from cancer comprising the step of: (i) measuring the expression level of IGFBP6 from a biological sample of said patient cell, wherein if a normal or decreased expression level of IGFBP6 relative to a control is present, administering a therapeutically acceptable amount of an IGF-1R/IR inhibitor to said patient.

The present invention also provides a method of identifying a treatment regimen for a patient suffering from cancer comprising the step of: (i) measuring the expression level of IGFBP6 from a biological sample of said patient cell, wherein if a normal or decreased expression level of IGFBP6 relative to a control is present, administering a therapeutically acceptable amount of an IGF-1R/IR inhibitor to said patient. Wherein said the cancer is a solid tumor, an advanced solid tumor, a metastatic solid tumor, a neoplasm, sarcoma, colon, and/or breast cancer, or other cancer outlined herein.

The present invention also provides a method of identifying a treatment regimen for a patient suffering from cancer comprising the step of: (i) measuring the expression level of IGFBP6 from a biological sample of said patient cell, wherein if an elevated expression level of IGFBP6 relative to a control is present, administering an IGF-1R/IR inhibitor in combination with one or more IGF-1R/IR inhibitors and/or one or more other agents.

The present invention also provides a method of identifying a treatment regimen for a patient suffering from cancer comprising the step of: (i) measuring the expression level of IGFBP6 from a biological sample of said patient cell, wherein if an elevated expression level of IGFBP6 relative to a control is present, administering an IGF-1R/IR inhibitor in combination with one or more IGF-1R/IR inhibitors and/or one or more other agents. Wherein said the cancer is a solid tumor, an advanced solid tumor, a metastatic solid tumor, a neoplasm, sarcoma, colon, and/or breast cancer, or other cancer outlined herein

The present invention also provides a method of identifying a treatment regimen for a patient suffering from cancer comprising the step of: (i) measuring the expression level of IGFBP6 from a biological sample of said patient cell, (ii) assessing the KRAS status of said patient, wherein if a normal or decreased expression level of IGFBP6 relative to a control is present in conjunction with a wild type KRAS, administering a therapeutically acceptable amount of an IGF-1R/IR inhibitor to said patient.

The present invention also provides a method of identifying a treatment regimen for a patient suffering from cancer comprising the step of: (i) measuring the expression level of IGFBP6 from a biological sample of said patient cell, (ii) assessing the KRAS status of said patient, wherein if a normal or decreased expression level of IGFBP6 relative to a control is present in conjunction with a wild type KRAS, administering a therapeutically acceptable amount of an IGF-1R/IR inhibitor to said patient. Wherein said the cancer is a solid tumor, an advanced solid tumor, a metastatic solid tumor, a neoplasm, sarcoma, colon, and/or breast cancer, or other cancer outlined herein.

The present invention also provides a method of identifying a treatment regimen for a patient suffering from cancer comprising the step of: (i) measuring the expression level of IGFBP6 from a biological sample of said patient cell, (ii) assessing the KRAS status of said patient, wherein if an elevated expression level of IGFBP6 relative to a control is present in conjunction with a wild type KRAS, administering an IGF-1R/IR inhibitor in combination with one or more IGF-1R/IR inhibitors and/or one or more other agents.

The present invention also provides a method of identifying a treatment regimen for a patient suffering from cancer comprising the step of: (i) measuring the expression level of IGFBP6 from a biological sample of said patient cell, (ii) assessing the KRAS status of said patient, wherein if an elevated expression level of IGFBP6 relative to a control is present in conjunction with a wild type KRAS, administering an IGF-1R/IR inhibitor in combination with one or more IGF-1R/IR inhibitors and/or one or more other agents. Wherein said the cancer is a solid tumor, an advanced solid tumor, a metastatic solid tumor, a neoplasm, sarcoma, colon, and/or breast cancer, or other cancer outlined herein.

The present invention also provides a method of identifying a treatment regimen for a patient suffering from cancer comprising the step of: (i) measuring the copy number of IRS2 from a biological sample of said patient cell, (ii) assessing the KRAS status of said patient, wherein if a normal or decreased copy number of IRS2 relative to a control is present in conjunction with a KRAS mutation, wherein if a normal or decreased copy number of IRS2 relative to a control is present in conjunction with a KRAS mutation, administering a therapeutically acceptable amount of an IGF-1R/IR inhibitor to said patient, administering a therapeutically acceptable amount of an IGF-1R/IR inhibitor to said patient.

The present invention also provides a method of identifying a treatment regimen for a patient suffering from cancer comprising the step of: (i) measuring the copy number of IRS2 from a biological sample of said patient cell, (ii) assessing the KRAS status of said patient, wherein if a normal or decreased copy number of IRS2 relative to a control is present in conjunction with a KRAS mutation, administering a therapeutically acceptable amount of an IGF-1R/IR inhibitor to said patient. Wherein said the cancer is a solid tumor, an advanced solid tumor, a metastatic solid tumor, a neoplasm, sarcoma, colon, and/or breast cancer, or other cancer outlined herein.

The present invention also provides a method of identifying a treatment regimen for a patient suffering from cancer comprising the step of: (i) measuring the copy number of IRS2 from a biological sample of said patient cell, (ii) assessing the KRAS status of said patient, wherein if a normal or decreased copy number of IRS2 relative to a control is present in conjunction with a KRAS mutation, wherein if a normal or decreased copy number of IRS2 relative to a control is present in addition to a wild type KRAS, administering an IGF-1R/IR inhibitor in combination with one or more IGF-1R/IR inhibitors and/or one or more other agents.

The present invention also provides a method of identifying a treatment regimen for a patient suffering from cancer comprising the step of: (i) measuring the copy number of IRS2 from a biological sample of said patient cell, (ii) assessing the KRAS status of said patient, wherein if a normal or decreased copy number of IRS2 relative to a control is present in conjunction with a KRAS mutation, wherein if a normal or decreased copy number of IRS2 relative to a control is present in addition to a wild type KRAS, or administering an IGF-1R/IR inhibitor in combination with one or more IGF-1R/IR inhibitors and/or one or more other agents. Wherein said the cancer is a solid tumor, an advanced solid tumor, a metastatic solid tumor, a neoplasm, sarcoma, colon, and/or breast cancer, or other cancer outlined herein.

The present invention also provides a method of identifying a treatment regimen for a patient suffering from cancer comprising the step of: (i) measuring the copy number of IRS2 from a biological sample of said patient cell, (ii) assessing the KRAS status of said patient, and if a normal or decreased copy number of IRS2 relative to a control is present in conjunction with a wild type KRAS, then (iii) measuring the expression level of IGFBP6, wherein if a normal or decreased expression level of IGFBP6 relative to a control is present, administering a therapeutically acceptable amount of an IGF-1R/IR inhibitor to said patient.

The present invention also provides a method of identifying a treatment regimen for a patient suffering from cancer comprising the step of: (i) measuring the copy number of IRS2 from a biological sample of said patient cell, (ii) assessing the KRAS status of said patient, and if a normal or decreased copy number of IRS2 relative to a control is present in conjunction with a wild type KRAS, then (iii) measuring the expression level of IGFBP6, wherein if a normal or decreased expression level of IGFBP6 relative to a control is present, administering a therapeutically acceptable amount of an IGF-1R/IR inhibitor to said patient. Wherein said the cancer is a solid tumor, an advanced solid tumor, a metastatic solid tumor, a neoplasm, sarcoma, colon, and/or breast cancer, or other cancer outlined herein.

The present invention also provides a method of identifying a treatment regimen for a patient suffering from cancer comprising the step of: (i) measuring the copy number of IRS2 from a biological sample of said patient cell, (ii) assessing the KRAS status of said patient, and if a normal or decreased copy number of IRS2 relative to a control is present in conjunction with a wild type KRAS, then (iii) measuring the expression level of IGFBP6, wherein if an elevated expression level of IGFBP6 relative to a control is present, administering an IGF-1R/IR inhibitor in combination with one or more IGF-1R/IR inhibitors and/or one or more other agents.

The present invention also provides a method of identifying a treatment regimen for a patient suffering from cancer comprising the step of: (i) measuring the copy number of IRS2 from a biological sample of said patient cell, (ii) assessing the KRAS status of said patient, and if a normal or decreased copy number of IRS2 relative to a control is present in conjunction with a wild type KRAS, then (iii) measuring the expression level of IGFBP6, wherein if an elevated expression level of IGFBP6 relative to a control is present, administering an IGF-1R/IR inhibitor in combination with one or more IGF-1R/IR inhibitors and/or one or more other agents. Wherein said the cancer is a solid tumor, an advanced solid tumor, a metastatic solid tumor, a neoplasm, sarcoma, colon, and/or breast cancer, or other cancer outlined herein.

The present invention also provides a method of identifying a treatment regiment for a patient suffering from colon cancer comprising the steps of: (a) measuring the copy number of IRS2 in a sample from said patient, and if said sample indicates said patent has an elevated or increased IRS2 copy number, (b) assessing the KRAS mutation status of said patient, and if said patient has either a KRAS mutation other than a G13D KRAS mutation, or is wild type KRAS, and (c) administering to said patient a therapeutically acceptable amount of an IGF-1R/IR inhibitor.

The present invention also provides a method of identifying a treatment regiment for a patient suffering from colon cancer comprising the steps of: (a) measuring the copy number of IRS2 in a sample from said patient, and if said sample indicates said patent has a normal or decreased IRS2 copy number, (b) assessing the KRAS status of said patient, and if said patient has a normal or decreased IRS2 copy number in conjunction with the presence of a wild type KRAS, further comprising the steps of (c) measuring the expression level of IGFBP6, and if said sample shows said patient has a normal or decreased expression level of IGFBP6, and (d) administering to said patient a therapeutically acceptable amount of an IGF-1R/IR inhibitor.

The present invention also provides a method of identifying a treatment regiment for a patient suffering from colon cancer comprising the steps of: (a) measuring the expression level of IGFBP6, wherein if said sample shows said patient has an reduce or decreased expression level of IGFBP6, (b) assessing the KRAS mutation status and BRAF mutation status of said patient, and if said patient has a decreased IGFBP6 expression level in conjunction with the presence of a wild type KRAS and wild type BRAF, and (c) administering to said patient a therapeutically acceptable amount of an IGF-1R inhibitor.

The present invention also provides a method of identifying a treatment regiment for a patient suffering from colon cancer comprising the steps of: (a) measuring the expression level of IGF1R, wherein if said sample shows said patient has an increased or elevated expression level of IGF1R, (b) assessing the KRAS mutation status and BRAF mutation status of said patient, and if said patient has an increased IGF1R expression level in conjunction with the presence of a wild type KRAS and wild type BRAF, and (c) administering to said patient a therapeutically acceptable amount of an IGF-1R inhibitor.

The present invention also provides a method of identifying a treatment regiment for a patient suffering from colon cancer comprising the steps of: (a) measuring the expression level of IR-A, wherein if said sample shows said patient has an increased or elevated expression level of IR-A, (b) assessing the KRAS mutation status and BRAF mutation status of said patient, and if said patient has an increased IR-A expression level in conjunction with the presence of a wild type KRAS and wild type BRAF, and (c) administering to said patient a therapeutically acceptable amount of an IGF-1R inhibitor.

The present invention also provides a method of treating a colon cancer patient comprising the step of: (i) measuring the copy number of IRS2 from a biological sample of said patient cell, wherein if elevated copy number of IRS2 is present, administering a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide to said patient.

The present invention also provides a method of treating a colon cancer patient comprising the step of: (i) measuring the copy number of IRS2 from a biological sample of said patient cell, wherein if elevated copy number of IRS2 is present, administering a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide to said patient. Wherein said the cancer is a solid tumor, an advanced solid tumor, a metastatic solid tumor, a neoplasm, sarcoma, colon, and/or breast cancer, or other cancer outlined herein.

The present invention also provides a method of treating a colon cancer patient comprising the step of: (i) measuring the copy number of IRS2 from a biological sample of said patient cell, and (ii) assessing the KRAS status of said patient, wherein if a normal or non-elevated copy number of IRS2 is present in conjunction with a KRAS mutation, administering a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide in combination with one or more IGF-1R/IR inhibitors and/or one or more other agents.

The present invention also provides a method of treating a colon cancer patient comprising the step of: (i) measuring the copy number of IRS2 from a biological sample of said patient cell, and (ii) assessing the KRAS status of said patient, wherein if a normal or non-elevated copy number of IRS2 is present in conjunction with a KRAS mutation, administering a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide in combination with one or more IGF-1R/IR inhibitors and/or one or more other agents. Wherein said the cancer is a solid tumor, an advanced solid tumor, a metastatic solid tumor, a neoplasm, sarcoma, colon, and/or breast cancer, or other cancer outlined herein.

The present invention also provides a method of treating a colon cancer patient comprising the step of: (i) measuring the expression level of IGFBP6 from a biological sample of said patient cell, wherein if a normal or decreased expression level of IGFBP6 relative to a control is present, administering a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide to said patient.

The present invention also provides a method of treating a colon cancer patient comprising the step of: (i) measuring the expression level of IGFBP6 from a biological sample of said patient cell, wherein if a normal or decreased expression level of IGFBP6 relative to a control is present, administering a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide to said patient. Wherein said the cancer is a solid tumor, an advanced solid tumor, a metastatic solid tumor, a neoplasm, sarcoma, colon, and/or breast cancer, or other cancer outlined herein.

The present invention also provides a method of treating a colon cancer patient comprising the step of: (i) measuring the expression level of IGFBP6 from a biological sample of said patient cell, wherein if an elevated expression level of IGFBP6 relative to a control is present, administering a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide in combination with one or more IGF-1R/IR inhibitors and/or one or more other agents.

The present invention also provides a method of treating a colon cancer patient comprising the step of: (i) measuring the expression level of IGFBP6 from a biological sample of said patient cell, wherein if an elevated expression level of IGFBP6 relative to a control is present, administering a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide in combination with one or more IGF-1R/IR inhibitors and/or one or more other agents. Wherein said the cancer is a solid tumor, an advanced solid tumor, a metastatic solid tumor, a neoplasm, sarcoma, colon, and/or breast cancer, or other cancer outlined herein

The present invention also provides a method of treating a colon cancer patient comprising the step of: (i) measuring the expression level of IGFBP6 from a biological sample of said patient cell, (ii) assessing the KRAS status of said patient, wherein if a normal or decreased expression level of IGFBP6 relative to a control is present in conjunction with a wild type KRAS, administering a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide to said patient.

The present invention also provides a method of treating a colon cancer patient comprising the step of: (i) measuring the expression level of IGFBP6 from a biological sample of said patient cell, (ii) assessing the KRAS status of said patient, wherein if a normal or decreased expression level of IGFBP6 relative to a control is present in conjunction with a wild type KRAS, administering a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide to said patient. Wherein said the cancer is a solid tumor, an advanced solid tumor, a metastatic solid tumor, a neoplasm, sarcoma, colon, and/or breast cancer, or other cancer outlined herein.

The present invention also provides a method of treating a colon cancer patient comprising the step of: (i) measuring the expression level of IGFBP6 from a biological sample of said patient cell, (ii) assessing the KRAS status of said patient, wherein if an elevated expression level of IGFBP6 relative to a control is present in conjunction with a wild type KRAS, administering a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide in combination with one or more IGF-1R/IR inhibitors and/or one or more other agents.

The present invention also provides a method of treating a colon cancer patient comprising the step of: (i) measuring the expression level of IGFBP6 from a biological sample of said patient cell, (ii) assessing the KRAS status of said patient, wherein if an elevated expression level of IGFBP6 relative to a control is present in conjunction with a wild type KRAS, administering a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide in combination with one or more IGF-1R/IR inhibitors and/or one or more other agents. Wherein said the cancer is a solid tumor, an advanced solid tumor, a metastatic solid tumor, a neoplasm, sarcoma, colon, and/or breast cancer, or other cancer outlined herein.

The present invention also provides a method of treating a colon cancer patient comprising the step of: (i) measuring the copy number of IRS2 from a biological sample of said patient cell, (ii) assessing the KRAS status of said patient, wherein if a normal or decreased copy number of IRS2 relative to a control is present in conjunction with a KRAS mutation, wherein if a normal or decreased copy number of IRS2 relative to a control is present in conjunction with a KRAS mutation, administering a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide to said patient, administering a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide to said patient.

The present invention also provides a method of treating a colon cancer patient comprising the step of: (i) measuring the copy number of IRS2 from a biological sample of said patient cell, (ii) assessing the KRAS status of said patient, wherein if a normal or decreased copy number of IRS2 relative to a control is present in conjunction with a KRAS mutation, administering a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide to said patient. Wherein said the cancer is a solid tumor, an advanced solid tumor, a metastatic solid tumor, a neoplasm, sarcoma, colon, and/or breast cancer, or other cancer outlined herein.

The present invention also provides a method of treating a colon cancer patient comprising the step of: (i) measuring the copy number of IRS2 from a biological sample of said patient cell, (ii) assessing the KRAS status of said patient, wherein if a normal or decreased copy number of IRS2 relative to a control is present in conjunction with a KRAS mutation, wherein if a normal or decreased copy number of IRS2 relative to a control is present in addition to a wild type KRAS, administering a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide in combination with one or more IGF-1R/IR inhibitors and/or one or more other agents.

The present invention also provides a method of treating a colon cancer patient comprising the step of: (i) measuring the copy number of IRS2 from a biological sample of said patient cell, (ii) assessing the KRAS status of said patient, wherein if a normal or decreased copy number of IRS2 relative to a control is present in conjunction with a KRAS mutation, wherein if a normal or decreased copy number of IRS2 relative to a control is present in addition to a wild type KRAS, or administering a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide in combination with one or more IGF-1R/IR inhibitors and/or one or more other agents. Wherein said the cancer is a solid tumor, an advanced solid tumor, a metastatic solid tumor, a neoplasm, sarcoma, colon, and/or breast cancer, or other cancer outlined herein.

The present invention also provides a method of treating a colon cancer patient comprising the step of: (i) measuring the copy number of IRS2 from a biological sample of said patient cell, (ii) assessing the KRAS status of said patient, and if a normal or decreased copy number of IRS2 relative to a control is present in conjunction with a wild type KRAS, then (iii) measuring the expression level of IGFBP6, wherein if a normal or decreased expression level of IGFBP6 relative to a control is present, administering a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide to said patient.

The present invention also provides a method of treating a colon cancer patient comprising the step of: (i) measuring the copy number of IRS2 from a biological sample of said patient cell, (ii) assessing the KRAS status of said patient, and if a normal or decreased copy number of IRS2 relative to a control is present in conjunction with a wild type KRAS, then (iii) measuring the expression level of IGFBP6, wherein if a normal or decreased expression level of IGFBP6 relative to a control is present, administering a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide to said patient. Wherein said the cancer is a solid tumor, an advanced solid tumor, a metastatic solid tumor, a neoplasm, sarcoma, colon, and/or breast cancer, or other cancer outlined herein.

The present invention also provides a method of treating a colon cancer patient comprising the step of: (i) measuring the copy number of IRS2 from a biological sample of said patient cell, (ii) assessing the KRAS status of said patient, and if a normal or decreased copy number of IRS2 relative to a control is present in conjunction with a wild type KRAS, then (iii) measuring the expression level of IGFBP6, wherein if an elevated expression level of IGFBP6 relative to a control is present, administering a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide in combination with one or more IGF-1R/IR inhibitors and/or one or more other agents.

The present invention also provides a method of treating a colon cancer patient comprising the step of: (i) measuring the copy number of IRS2 from a biological sample of said patient cell, (ii) assessing the KRAS status of said patient, and if a normal or decreased copy number of IRS2 relative to a control is present in conjunction with a wild type KRAS, then (iii) measuring the expression level of IGFBP6, wherein if an elevated expression level of IGFBP6 relative to a control is present, administering a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide in combination with one or more IGF-1R/IR inhibitors and/or one or more other agents. Wherein said the cancer is a solid tumor, an advanced solid tumor, a metastatic solid tumor, a neoplasm, sarcoma, colon, and/or breast cancer, or other cancer outlined herein.

The present invention also provides a method of treating a colon cancer patient comprising the steps of: (a) measuring the copy number of IRS2 in a sample from said patient, and if said sample indicates said patent has an elevated or increased IRS2 copy number, (b) assessing the KRAS mutation status of said patient, and if said patient has either a KRAS mutation other than a G13D KRAS mutation, or is wild type KRAS, and (c) administering to said patient a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide.

The present invention also provides a method of treating a colon cancer patient comprising the steps of: (a) measuring the copy number of IRS2 in a sample from said patient, and if said sample indicates said patent has a normal or decreased IRS2 copy number, (b) assessing the KRAS status of said patient, and if said patient has a normal or decreased IRS2 copy number in conjunction with the presence of a wild type KRAS, further comprising the steps of (c) measuring the expression level of IGFBP6, and if said sample shows said patient has a normal or decreased expression level of IGFBP6, and (d) administering to said patient a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide.

The present invention also provides a method of treating a colon cancer patient comprising the steps of: (a) measuring the expression level of IGFBP6, wherein if said sample shows said patient has an reduce or decreased expression level of IGFBP6, (b) assessing the KRAS mutation status and BRAF mutation status of said patient, and if said patient has a decreased IGFBP6 expression level in conjunction with the presence of a wild type KRAS and wild type BRAF, and (c) administering to said patient a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide.

The present invention also provides a method of treating a colon cancer patient comprising the steps of: (a) measuring the expression level of IGF1R, wherein if said sample shows said patient has an increased or elevated expression level of IGF1R, (b) assessing the KRAS mutation status and BRAF mutation status of said patient, and if said patient has an increased IGF1R expression level in conjunction with the presence of a wild type KRAS and wild type BRAF, and (c) administering to said patient a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide.

The present invention also provides a method of treating a colon cancer patient comprising the steps of: (a) measuring the expression level of IR-A, wherein if said sample shows said patient has an increased or elevated expression level of IR-A, (b) assessing the KRAS mutation status and BRAF mutation status of said patient, and if said patient has an increased IR-A expression level in conjunction with the presence of a wild type KRAS and wild type BRAF, and (c) administering to said patient a therapeutically acceptable amount of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide.

The diagnostic methods of the invention can be, for example, an in vitro method wherein the step of measuring in the mammal the level of at least one biomarker comprises taking a biological sample from the mammal and then measuring the level of the biomarker(s) in the biological sample. The biological sample can comprise, for example, at least one of serum, whole fresh blood, peripheral blood mononuclear cells, frozen whole blood, fresh plasma, frozen plasma, urine, saliva, skin, hair follicle, bone marrow, tumor tissue, tumor biopsy, or archived paraffin-embedded tumor tissue.

The status or level of the at least one biomarker can be, for example, at the level of DNA, protein and/or mRNA transcript of the biomarker(s).

The invention also provides an isolated IRS2 biomarker, an isolated IGFBP6 biomarker, IR-A, IGF1R, BRAF, PI3KCA, and KRAS mutation biomarkers. The biomarkers of the invention may also include nucleotide and/or amino acid sequences that are at least 90%, 95%, 96%, 97%, 98%, 99%, and 100% identical to the sequences provided as gi|NP_{—}003740 (SEQ ID NO:1 and 2) (IRS2); gi|NM_{—}002178 (SEQ ID NO:3 and 4) (IGFBP6); gi|NM_{—}000208 (SEQ ID NO:5 and 6) (IR-A); gi|NM_{—}000208 (SEQ ID NO:7 and 8) (IGF-1R) as well as fragments, naturally occurring variants, mutants, and variants thereof.

The invention also provides a biomarker set comprising two or more biomarkers of the invention.

The invention also provides kits for measuring copy number of IRS2, for measuring KRAS mutant status, for measuring BRAF mutation status, for measuring PI3KCA mutation status, expression of IR-A, expression of IGF1R, and/or expression of IGFBP6.

The present invention provides a kit for use in treating a patient with cancer, comprising: (a) a means for measuring IRS2 copy number; (b) a therapeutically effective amount of an IGF-1R/IR inhibitor; and instructions to administer said IGF-1R/IR inhibitor if elevated IRS2 copy number is present.

The present invention provides a kit for use in treating a patient with cancer, comprising: (a) a means for measuring IRS2 copy number; (b) a means for measuring KRAS mutation status; (c) a therapeutically effective amount of an IGF-1R/IR inhibitor; and instructions to administer said IGF-1R/IR inhibitor if normal or decreased IRS2 copy number is detected in conjunction with wild type KRAS is present.

The present invention provides a kit for use in treating a patient with cancer, comprising: (a) a means for measuring IRS2 copy number; (b) a means for measuring KRAS mutation status; (c) a means for measuring IGFBP6 expression; and (d) a therapeutically effective amount of an IGF-1R/IR inhibitor; and instructions to administer said IGF-1R/IR inhibitor if normal or decreased IRS2 copy number is detected in conjunction with wild type KRAS and normal or decreased IGFBP6 expression is present.

The present invention provides a kit for use in treating a patient with cancer, comprising: (a) a means for measuring IRS2 copy number; (b) a means for measuring KRAS mutation status; (c) a therapeutically effective amount of an IGF-1R/IR inhibitor in combination with one or more additional agents; and instructions to administer a more aggressive treatment regimen of said IGF-1R/IR inhibitor either alone or in combination with one or more additional agents if normal or decreased IRS2 copy number is detected in conjunction with wild type KRAS mutant is present.

The present invention provides a kit for use in treating a patient with cancer, comprising: (a) a means for measuring IRS2 copy number; (b) a means for measuring KRAS mutation status; (c) a means for measuring IGFBP6 expression (d) a therapeutically effective amount of an IGF-1R/IR inhibitor in combination with one or more additional agents; and instructions to administer a more aggressive treatment regimen of said IGF-1R/IR inhibitor either alone or in combination with one or more additional agents if normal or decreased IRS2 copy number is detected in conjunction with wild type KRAS mutant and elevated IGFBP6 expression is present.

The present invention provides a kit for use in treating a patient with cancer, comprising: (a) a means for measuring the IRS2 copy number in a patient sample; (b) a means for determining the KRAS mutation status of said patient sample or a means for determining the BRAF mutation status; and (c) a therapeutically effective amount of an IGF-1R inhibitor, and instructions for administering said IGF-1R inhibitor if said patient has wild type KRAS or KRAS mutation other than a G13D mutation, a wild type BRAF, and has an increased or elevated IRS2 copy number.

The present invention provides a kit for use in treating a patient with cancer, comprising: (a) a means for measuring the BRAF mutation status in a patient sample; (b) a means for determining the KRAS mutation status of said patient sample; (c) a means for measuring the IGFBP6, IR-A, or IGF1R expression level in a patient sample, and (d) a therapeutically effective amount of an IGF-1R/IR inhibitor, and instructions for administering said IGF-1R/IR inhibitor if said patient is KRAS wild type, is BRAF wild type, and has either a decreased IGFBP6 expression level, or an increased IGF1R or IR-A level.

The present invention provides a method according to any of the embodiments outlined herein wherein said measurement is performed using a method selected from the group consisting of: (a) PCR; (b) RT-PCR; (c) FISH; (d) IHC; (e) immunodetection methods; (f) Western Blot; (g) ELISA; (h) radioimmuno assays; (i) immunoprecipitation; (j) FACS (k) HPLC; (l) surface plasmon resonance; (m) optical spectroscopy; and (n) mass spectrometry.

The present invention provides a method according to any of the embodiments outlined herein, wherein said cancer is a solid tumor, a metastatic tumor, colon cancer, breast cancer or lung cancer.

The invention will be better understood upon a reading of the detailed description of the invention when considered in connection with the accompanying figures.

### BRIEF DESCRIPTION OF THE DRAWINGS

FIG. 1 shows the results of a DNA copy number analysis using Affymetrix SNP 6 arrays in which it is demonstrated that chromosome 17 amplification was found in a subset of 43 CRC cell lines which correlated with sensitivity to IGF-1R/IR inhibition by BMS-754807. Cell lines are rank ordered by IC₅₀, and the position of IRS2 is indicated.

FIGS. 2A-C shows the correlation between IRS-2 copy number (A), mRNA expression levels (B), protein expression level (C), and the sensitivity to IGF-1R/IR inhibition by BMS-754807 in CRC cell line panel.

FIGS. 3A-B show RNA expression levels of IGF-1R/IR (A) and IGFBP6 (B) in the CRC cell line panel, both correlate with the sensitivity to IGF-1R/IR by BMS-754807.

FIGS. 4A-C show (A) IRS2 DNA amplification, IGFBP6 RNA expression level and KRAS status in a panel of CRC cell lines and their correlation with BMS-754807 sensitivity; (B) Status of IRS2 DNA amplification by KRAS status; and (C) IGFBP6 RNA expression level by KRAS status.

FIGS. 5A-D show the correlation between sensitivity to BMS-754807 and predictive biomarkers in a panel of CRC cell lines. (A) Sensitivity classification of BMS-754807 as measured by an in vitro proliferation MTS assay. Cell lines with IC₅₀≦50 nmol/L were defined as sensitive and the ones with IC₅₀>50 nmol/L were defined as resistant. (B) The mutational status of KRAS, BRAF and PIK3CA, and their correlation with sensitivity to BMS-754807. On the left are the p values from the Fisher exact test. (C) The heat map of 197 genes on chromosome 13 with variation in DNA copy number significantly associated with the drug sensitivity. (D) Representative examples of IRS2 copy number examined by FISH analysis in CRC cell lines: IRS2 (red), RB1 (green) and chromosome 10 satellite enumeration (SE10, blue) probes were hybridized to the interphase nuclei of cells. SW403 and SK-00-1 have IRS2 amplification; HT-15 and SW837 have normal copy number of IRS2.

FIGS. 6A-G show (A) box-plots for the mean levels and distribution of IRS2 DNA copy numbers (Top), RNA (Middle) and protein (Bottom) expression levels between the sensitive and resistant cell lines to BMS-754807. The p values are from t-tests. (B) The distribution of number of cell lines between BMS-754807 sensitive and resistant groups in KRAS mutated, BRAF mutated, and KRAS/BRAF-WT subpopulations. (C) Correlation between IRS2 amplification and BMS-754807 sensitivity in KRAS mutated CRC cell lines. (D) IRS2 amplification and BRAF mutational status, and correlation to BMS-754807 sensitivity in KRAS-WT CRC cell lines. (E) RNA expression patterns of IGF-1R (as measured by Affymetrix GENECHIP®) between sensitive and resistant groups in KRAS mutated CRC cell lines. (F) RNA expression patterns of IR-A isoform (as measured by qRT-PCR and normalized to (3-actin) in KRAS/BRAF-WT CRC cell lines. (G) RNA expression patterns of IGFBP6 (as measured by Affymetrix GENECHIP®) between sensitive and resistant groups in KRAS/BRAF-WT CRC cell lines.

FIGS. 7A-D show IGF-1R/IR pathway signaling in CRC cell lines. Correlation between IRS2 DNA copy number and ligand-stimulated activation of IGF-1R (A) and AKT (B) in 60 CRC cell lines. Cells were serum-starved overnight, then either stimulated with 50 ng/ml IGF-1, IGF-2 or insulin for 10 minutes, or unstimulated. Cell lysates were subjected to MSD for measuring both total and phosphorylated IGF-1R and AKT. The phospho-protein values were normalized to the corresponding total protein levels (e.g., pIGF-1R/IGF-1R). The ligand-stimulated activation of IGF-1R or AKT is presented as the ratio of the pIGF-1R/IGF-1R or pAKT/AKT value in IGF-1, IGF-2 or insulin-stimulated cells vs. the value in the non-stimulated cells. Activation of IGF-1R (A) or AKT (B) stimulated by IGF-1 (top panel), by IGF-2 (middle panel), or by insulin (bottom panel); Cell lines ordered by IRS2 DNA copy number. (C) Western blot analysis for IRS2, pIGF-1R/pIR, pAKT, pMAPK and ACTIN® in SK-CO-1 cell line (KRAS^{G12v}, IRS2 DNA copy number=3, IC₅₀=0.003 μM). (D) Western blot analysis for IRS2, pIGF-1R/pIR, pAKT, pMAPK and ACTIN® in DLD-1 cell line (KRAS^{G13(D)} IRS2 DNA copy number=2.2, IC₅₀=0.666 μM). Cells were cultured in medium containing 10% FBS overnight, then untreated or treated with 10 or 100 nM of BMS-754807 for 1 hour, followed by 50 ng/ml IGF-1, IGF-2 or insulin stimulation for 10 minutes. 20 ug of total protein from cell lysates were subjected to Western blot analysis.

FIGS. 8A-B show modulation of IRS2 expression levels change the sensitivity to BMS-754807 in COLO320DM, LS-513 and SW403 CRC cell lines. (A) IRS2 siRNA in CRC cell lines significantly reduces the expression levels of IRS2 as shown by Western blot compared to non-targeting siRNA control and untransfected cells. (B) knockdown of IRS2 decreased the sensitivity to BMS-754807 as measured by MTS proliferation assay and led to a shift to the right in the IC₅₀ curves (data are graphed as mean percent of control with SD).

FIGS. 9A-C show (A) a diagram depicting the predictive classification of responsiveness to BMS-754807 in KRAS mutants and WT subpopulations. The numbers refer the prediction score. Sensitivity class (true and predicted): light=sensitive; dark=resistant. The true sensitivity class is based on IC₅₀ value (cell lines with IC₅₀=<50 nmol/L were defined as sensitive and the ones with IC₅₀>50 nmol/L were defined as resistant) and the predicted class is based on the overall score of biomarkers. If a cell line with the sum of score>=2, it was classified to be sensitive; if the sum of score <2, it was classified to be resistant. Arrows indicate where the classification prediction was in error. KRAS mutation: G13D mutation (score=0); other KRAS mutations (score=1). IRS2 amplification: amplified (score=1); normal copy number (score=0). IGF-1R or IR-A RNA expression: high expression level (>=Mean, score=1); low expression level (<Mean, score=1). IGFBP6 RNA expression: high expression level (>=Mean, score=0); low expression level (<Mean, score=1). The “Mean” refers the average expression level for IGF-1R, IR-A or IGFBP6 across all 60 CRC cell lines and was used as the cut-off value for response prediction for each biomarker respectively. (B) Schematic illustration of possible mechanisms for sensitivity and resistance to IGF-1R/IR TKI. Left: cells with activated IGF-1R/IR pathway via IRS2 amplification, high expression of IGF-1R or IR-A, low expression of IGFBP6, were more dependent on IGF-1R/IR pathways as the predominant driver for activation of AKT and ERK, making them more sensitive to IGF-1R/IR TKI inhibition which consequently causes inhibition of downstream effectors, and cell proliferation. Right: cells without activation of IGF-1R/IR pathway, but with KRAS, PIK3CA, or BRAF mutations lead to dysregulation of MAPK and AKT pathways and are less dependent on IGF-1R/IR pathway for proliferation; inhibition of IGF-1R/IR activity was not sufficient enough to inhibit cell proliferation, therefore resistance to IGF-1R/IR TKI. (C) Illustration of how the biomarkers KRAS and BRAF mutations, IRS2 copy number, IGF-1R, IR-A and IGFBP6 RNA expression levels can be used to predict response to the IGF-1R/IR inhibitor BMS-754807 in CRC.

FIGS. 10A-B show CNV profiles of CRC cell lines (A) and CRC tumors (B) were plotted using the Kcsmart package in the Bioconductor project. Copy number gain indicated by peaks in the top row and copy number deletion or loss indicated by peaks in the bottom row.

FIGS. 11A-F show the mean levels comparison for IRS2 DNA copy number (A), for IGF-1R RNA expression (B), for IR RNA expression (C), for IR-A isoform RNA expression (D), for IGFBP6 RNA expression (E), and for IGF2BP3 RNA expression (F) between the sensitive and resistant cell lines in all, KRAS mutants, KRAS/BRAF-WT/WT population.

FIGS. 12A-C show box-plots to compare IRS2 DNA copy number, IRS2 protein and IGF-1R RNA expression levels between cell lines with KRAS^{G13D} mutation and cell lines with other KRAS mutations. P values are from t-tests.

FIG. 13 shows IRS2 DNA amplification, and sensitivity to BMS-754807 were both inversely correlated with the basal level of MAPK activation (phosphorylated MAPK normalized to total MAPK) in a panel of 60 CRC cell lines (sensitive cell lines are highlighted). Cell lysates from cell lines were subjected for MSD for both total and phosphorylated MAPK analysis. The data was presented as the ratio of pMAPK/MAPK for basal level activation. P values are from t-tests to compare IRS2 amplified lines with non-amplified lines; as well as compare sensitive cell lines to resistant ones.

FIGS. 14A-B provide graphical representations of some of the data shown in FIGS. 5A-D to illustrate the correlation between A. IRS2 and IGFBP6 with sensitivity to BMS-754807; and IR-A with sensitivity to BMS-754807.

### DETAILED DESCRIPTION OF THE INVENTION

The present invention relates to the identification of markers for predicting resistance, partial resistance, or sensitivity to IGF-1R/IR therapy prior to or concurrent with treatment, in addition to methods of treating patients with such resistance or such sensitivity in addition to treatment regimens related thereto.

Specifically, the present inventors carried out in vitro studies with BMS-754807 and determined that it was active in a subset of colorectal cancer (CRC) cell lines tested by cellular proliferation assay. As a result, they were able to determine that CRC provides a suitable model system for identification of predictive biomarker for BMS-754807 that can be used to select the patient population most likely to benefit from the therapy. As a result, disclosed herein are the results of several genomic approaches including DNA copy number variations, mutation, gene expression etc. for predictive biomarker discovery, the results of which provide potential several patient stratification strategies for IGF-IR inhibitors that can be clinical tested and validated.

As is known in the art, BMS-754807 refers to a compound having the following structure (I):

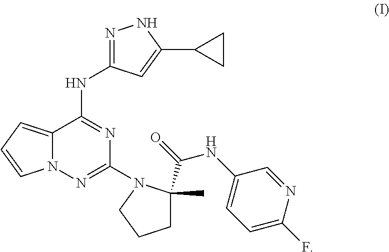 [SMILES: C[C@@]1(C(=O)Nc2ccc(F)nc2)CCCN1c1nc(Nc2cc(C3CC3)[nH]n2)c2cccn2n1]

Compound (I) is also referred to as (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide in accordance with IUPAC nomenclature. Use of the term “(2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide” encompasses (unless otherwise indicated) solvates (including hydrates) and polymorphic forms of the compound (I) or its salts, such as the forms of (I) described in U.S. Pat. No. 7,534,792, U.S. Pat. No. 7,879,855 and/or PCT Publication No. WO 2011/097331, which are incorporated herein by reference in their entirety and for all purposes. Pharmaceutical compositions of (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide include all pharmaceutically acceptable compositions comprising (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide and one or more diluents, vehicles and/or excipients One example of a pharmaceutical composition comprising (2S)-1-(4-((5-cyclopropyl-1H-pyrazol-3-yl)amino)pyrrolo[2,1-f]]1,2,4]triazin-2-yl)-N-(6-fluoro-3-pyridinyl)-2-methyl-2-pyrrolidinecarboxamide is BMS-754807 (Bristol-Myers Squibb Company). BMS-754807 comprises (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide as the active ingredient.

In one aspect, the IGF-1R/IR modulator is an IGF-1R/IR antibody provided in PCT Publication Nos. WO 2005/016970, WO 02/53596, WO 2004/71529, WO 2005/16967, WO 2004/83248, WO 03/106621, WO 03/100008, WO 03/59951, WO 2004/87756, or WO 2005/05635.

In another aspect, the IGF-1R/IR modulator is derived from fibronectin, such as an ADNECTIN™ (Adnexus Therapeutics) (See, PCT Publication Nos. WO 00/34784, WO 01/64942, WO 02/32925).

The term “EGFR inhibitor” refers to a small molecule, antibody, siRNA, adnectins, domain antibody, or other molecule capable of inhibiting the expression and/or activity of EGFR, either at the DNA level or protein level, and either inhibiting the kinase activity of EGFR or the ability of EGF to bind to EGFR, among other activities. Examples of an EGFR inhibitor include the examples provided in the paragraphs that follow in addition to the foregoing: EGFR antibodies that may be chimerized, humanized, fully human, and single chain antibodies derived from the murine antibody 225 described in U.S. Pat. No. 4,943,533.

In another aspect, the EGFR inhibitor is cetuximab (IMC-C225) which is a chimeric (human/mouse) IgG monoclonal antibody, also known under the tradename ERBITUX®. Cetuximab Fab contains the Fab fragment of cetuximab, i.e., the heavy and light chain variable region sequences of murine antibody M225 (U.S. Application No. 2004/0006212, incorporated herein by reference) with human IgG1 C_{H}1 heavy and kappa light chain constant domains. Cetuximab includes all three IgG1 heavy chain constant domains.

In another aspect, the EGFR inhibitor can be selected from the antibodies described in U.S. Pat. Nos. 6,235,883, 5,558,864, and 5,891,996. The EGFR antibody can be, for example, AGX-EGF (Amgen Inc.) (also known as panitumumab) which is a fully human IgG2 monoclonal antibody. The sequence and characterization of ABX-EGF, which was formerly known as clone E7.6.3, is disclosed in U.S. Pat. No. 6,235,883 at column 28, line 62 through column 29, line 36 and FIGS. 29-34, which is incorporated by reference herein. The EGFR antibody can also be, for example, EMD72000 (Merck KGaA), which is a humanized version of the murine EGFR antibody EMD 55900. The EGFR antibody can also be, for example: h-R3 (TheraCIM), which is a humanized EGFR monoclonal antibody; Y10 which is a murine monoclonal antibody raised against a murine homologue of the human EGFRvIII mutation; or MDX-447 (Medarex Inc.).

In addition to the biological molecules discussed above, the EGFR modulators useful in the invention may also be small molecules. Any molecule that is not a biological molecule is considered herein to be a small molecule. Some examples of small molecules include organic compounds, organometallic compounds, salts of organic and organometallic compounds, saccharides, amino acids, and nucleotides. Small molecules further include molecules that would otherwise be considered biological molecules, except their molecular weight is not greater than 450. Thus, small molecules may be lipids, oligosaccharides, oligopeptides, and oligonucleotides and their derivatives, having a molecular weight of 450 or less.

It is emphasized that small molecules can have any molecular weight. They are merely called small molecules because they typically have molecular weights less than 450. Small molecules include compounds that are found in nature as well as synthetic compounds. In one embodiment, the EGFR modulator is a small molecule that inhibits the growth of tumor cells that express EGFR. In another embodiment, the EGFR modulator is a small molecule that inhibits the growth of refractory tumor cells that express EGFR.

Numerous small molecules have been described as being useful to inhibit EGFR.

One example of a small molecule EGFR antagonist is IRESSA® (ZD1939), which is a quinozaline derivative that functions as an ATP-mimetic to inhibit EGFR. See, U.S. Pat. No. 5,616,582; PCT Publication No. WO 96/33980 at page 4. Another example of a small molecule EGFR antagonist is TARCEVA® (OSI-774), which is a 4-(substituted phenylamino)quinozaline derivative [6,7-bis(2-methoxy-ethoxy)-quinazolin-4-yl]-(3-ethynyl-1-phenyl)amine hydrochloride] EGFR inhibitor. See PCT Publication No. WO 96/30347 (Pfizer Inc.) at, for example, page 2, line 12 through page 4, line 34 and page 19, lines 14-17. TARCEVA® may function by inhibiting phosphorylation of EGFR and its downstream PI3/Akt and MAP (mitogen activated protein) kinase signal transduction pathways resulting in p27-mediated cell-cycle arrest. See Hidalgo et al., Abstract 281 presented at the 37th Annual Meeting of ASCO, San Francisco, Calif., May 12-15, 2001.

Other small molecules are also reported to inhibit EGFR, many of which are thought to be specific to the tyrosine kinase domain of an EGFR. Some examples of such small molecule EGFR antagonists are described in PCT Publication Nos. WO 91/116051, WO 96/30347, WO 96/33980, WO 97/27199. WO 97/30034, WO 97/42187, WO 97/49688, WO 98/33798, WO 00/18761, and WO 00/31048. Examples of specific small molecule EGFR antagonists include C1-1033 (Pfizer Inc.), which is a quinozaline (N-[4-(3-chloro-4-fluoro-phenylamino)-7-(3-morpholin-4-yl-propoxy)-quinazolin-6-yl]-acrylamide) inhibitor of tyrosine kinases, particularly EGFR and is described in WO 00/31048 at page 8, lines 22-6; PKI166 (Novartis), which is a pyrrolopyrimidine inhibitor of EGFR and is described in WO 97/27199 at pages 10-12; GW2016 (GlaxoSmithKline), which is an inhibitor of EGFR and HER2; EKB569 (Wyeth), which is reported to inhibit the growth of tumor cells that overexpress EGFR or HER2 in vitro and in vivo; AG-1478 (Tryphostin), which is a quinazoline small molecule that inhibits signaling from both EGFR and erbB-2; AG-1478 (Sugen), which is a bisubstrate inhibitor that also inhibits protein kinase CK2; PD 153035 (Parke-Davis) which is reported to inhibit EGFR kinase activity and tumor growth, induce apoptosis in cells in culture, and enhance the cytotoxicity of cytotoxic chemotherapeutic agents; SPM-924 (Schwarz Pharma), which is a tyrosine kinase inhibitor targeted for treatment of prostate cancer; CP-546,989 (OSI Pharmaceuticals), which is reportedly an inhibitor of angiogenesis for treatment of solid tumors; ADL-681, which is a EGFR kinase inhibitor targeted for treatment of cancer; PD 158780, which is a pyridopyrimidine that is reported to inhibit the tumor growth rate of A4431 xenografts in mice; CP-358,774, which is a quinzoline that is reported to inhibit autophosphorylation in HN5 xenografts in mice; ZD1839, which is a quinzoline that is reported to have antitumor activity in mouse xenograft models including vulvar, NSCLC, prostrate, ovarian, and colorectal cancers; CGP 59326A, which is a pyrrolopyrimidine that is reported to inhibit growth of EGFR-positive xenografts in mice; PD 165557 (Pfizer); CGP54211 and CGP53353 (Novartis), which are dianilnophthalimides. Naturally derived EGFR tyrosine kinase inhibitors include genistein, herbimycin A, quercetin, and erbstatin.

Further small molecules reported to inhibit EGFR and that are therefore within the scope of the present invention are tricyclic compounds such as the compounds described in U.S. Pat. No. 5,679,683; quinazoline derivatives such as the derivatives described in U.S. Pat. No. 5,616,582; and indole compounds such as the compounds described in U.S. Pat. No. 5,196,446.

Further small molecules reported to inhibit EGFR and that are therefore within the scope of the present invention are styryl substituted heteroaryl compounds such as the compounds described in U.S. Pat. No. 5,656,655. The heteroaryl group is a monocyclic ring with one or two heteroatoms, or a bicyclic ring with 1 to about 4 heteroatoms, the compound being optionally substituted or polysubstituted.

Further small molecules reported to inhibit EGFR and that are therefore within the scope of the present invention are bis mono and/or bicyclic aryl heteroaryl, carbocyclic, and heterocarbocyclic compounds described in U.S. Pat. No. 5,646,153.

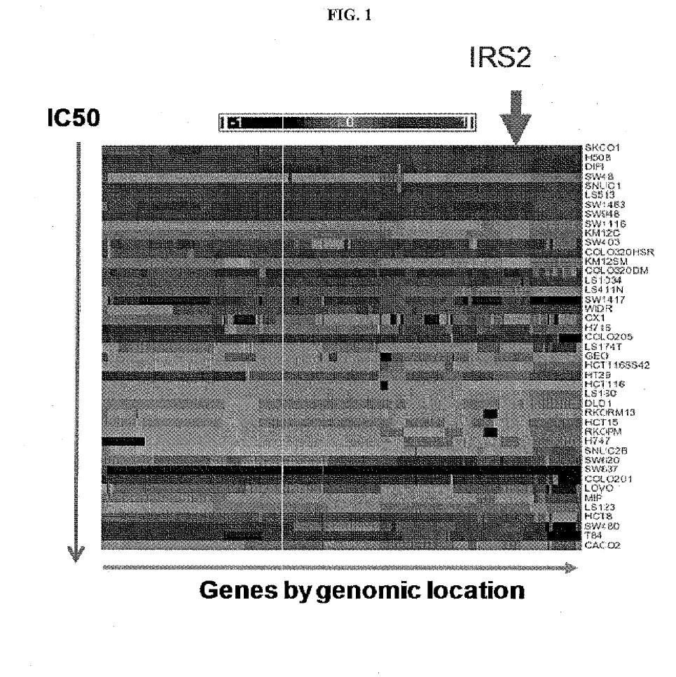

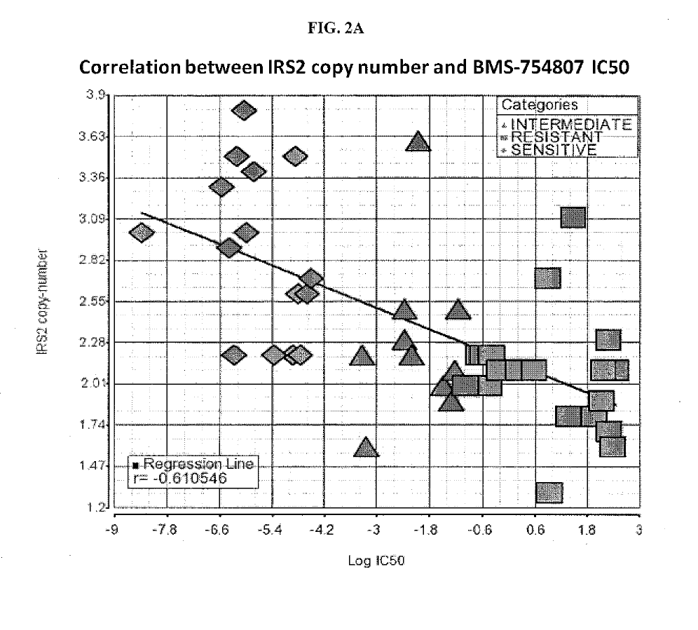

Further small molecules reported to inhibit EGFR and that are therefore within the scope of the present invention is the compound provided FIG. 1 of Fry et al., Science, 265:1093-1095 (1994) that inhibits EGFR.

Further small molecules reported to inhibit EGFR and that are therefore within the scope of the present invention are tyrphostins that inhibit EGFR/HER1 and HER 2, particularly those in Tables I, II, III, and IV described in Osherov et al., J. Biol. Chem., 268(15):11134-11142 (1993).

Further small molecules reported to inhibit EGFR and that are therefore within the scope of the present invention is a compound identified as PD166285 that inhibits the EGFR, PDGFR, and FGFR families of receptors. PD166285 is identified as 6-(2,6-dichlorophenyl)-2-(4-(2-diethylaminoethyoxy)phenylamino)-8-methyl-8H-pyrido(2,3-d)pyrimidin-7-one having the structure shown in FIG. 1 on page 1436 of Panek et al., J. Pharmacol. Exp. Ther., 283:1433-1444 (1997).

In addition to the biological molecules discussed above, the IGF1R modulators useful in the invention may also be small molecules. Any molecule that is not a biological molecule is considered herein to be a small molecule. Some examples of small molecules include organic compounds, organometallic compounds, salts of organic and organometallic compounds, saccharides, amino acids, and nucleotides. Small molecules further include molecules that would otherwise be considered biological molecules, except their molecular weight is not greater than 450. Thus, small molecules may be lipids, oligosaccharides, oligopeptides, and oligonucleotides and their derivatives, having a molecular weight of 450 or less.

It is emphasized that small molecules can have any molecular weight. They are merely called small molecules because they typically have molecular weights less than 450. Small molecules include compounds that are found in nature as well as synthetic compounds. In one embodiment, the IGF1R modulator is a small molecule that inhibits the growth of tumor cells that express IGF1R. In another embodiment, the IGF1R modulator is a small molecule that inhibits the growth of refractory tumor cells that express IGF1R.

Numerous small molecules have been described as being useful to inhibit IGF1R.

For the purposes of the present invention, small molecule IGF-1R/IR inhibitors may also include small molecules, adnectins, siRNAs, iRNA, and antisense molecules.

In one aspect, the IGF1R modulator is selected from PCT Publication Nos. WO 02/79192, WO 2004/30620, WO 2004/31401 WO 2004/63151, and WO 2005/21510, and from U.S. Provisional Application Nos. 60/819,171, 60/870,872, 60/883,601, and 60/912,446.

In another aspect, the IGF-1R/IR modulator is selected from (S)-4-(2-(3-chlorophenyl)-2-hydroxyethylamino)-3-(4-methyl-6-morpholino-1H-benzo[d]imidazol-2-yl)-pyridin-2(1-H)-one and (2S)-1-(4-((5-cyclopropyl-1H-pyrazol-3-yl)amino)pyrrolo[2,1-f][1,2,4]triazin-2-yl)-N-(6-fluoro-3-pyridinyl)-2-methyl-2-pyrrolidinecarboxamide.

In another aspect, the IGF-1R/IR modulator is selected from XL-228 (Exelixis), AEW-541 (Novartis), and OSI-906 (OSI).

The phrase “microtubulin modulating agent” is meant to refer to agents that either stabilize microtubulin or destabilize microtubulin synthesis and/or polymerization.

Microtubulin modulatory agents either agonize or inhibit a cells ability to maintain proper microtubulin assemblies. In the case of paclitaxel (marketed as TAXOL®) causes mitotic abnormalities and arrest, and promotes microtubule assembly into calcium-stable aggregated structures resulting in inhibition of cell replication.

Epothilones mimic the biological effects of TAXOL®, (Bollag et al., Cancer Res., 55:2325-2333 (1995), and in competition studies act as competitive inhibitors of TAXOL® binding to microtubules. However, epothilones enjoy a significant advantage over TAXOL® in that epothilones exhibit a much lower drop in potency compared to TAXOL® against a multiple drug-resistant cell line (Bollag et al. (1995)). Furthermore, epothilones are considerably less efficiently exported from the cells by P-glycoprotein than is TAXOL® (Gerth et al. (1996)).

Ixabepilone is a semi-synthetic lactam analogue of patupilone that binds to tubulin and promotes tubulin polymerization and microtubule stabilization, thereby arresting cells in the G2/M phase of the cell cycle and inducing tumor cell apoptosis.

Thus, in one embodiment, the therapeutic method of the invention comprises the administration of an epothilone in combination with an IGF-1R/IR inhibitor.

Combinations of an IGF-1R/IR inhibitor with another agent is contemplated by the present invention, and may include the addition of an anti-proliferative cytotoxic agent. Classes of compounds that may be used as anti-proliferative cytotoxic agents include the following: co-stimulatory modulating agents including, without limitation, CTLA4 antagonists, ipilimumab, agatolimod, belatacept, blinatumomab, CD40 ligand, anti-B7-1 antibody, anti-B7-2 antibody, anti-B7-H4 antibody, AG4263, eritoran, anti-OX40 antibody, ISF-154, and SGN-70; EGFR inhibitors (including, without limitation, Erbitux®); microtubulin stabilizing agents, (including, without limitation, TAXOL®); alkylating agents (including, without limitation, nitrogen mustards, ethylenimine derivatives, alkyl sulfonates, nitrosoureas and triazenes): Uracil mustard, Chlormethine, Cyclophosphamide (CYTOXAN®), Ifosfamide, Melphalan, Chlorambucil, Pipobroman, Triethylene-melamine, Triethylenethiophosphoramine, Busulfan, Carmustine, Lomustine, Streptozocin, Dacarbazine, and Temozolomide; antimetabolites (including, without limitation, folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gemcitabine; and natural products and their derivatives (for example, vinca alkaloids, antitumor antibiotics, enzymes, lymphokines and epipodophyllotoxins): Vinblastine, Vincristine, Vindesine, Bleomycin, Dactinomycin, Daunorubicin, Doxorubicin, Epirubicin, Idarubicin, Ara-C, paclitaxel (paclitaxel is commercially available as TAXOL®), Mithramycin, Deoxyco-formycin, Mitomycin-C, L-Asparaginase, Interferons (especially IFN-a), Etoposide, and Teniposide.

Other anti-proliferative cytotoxic agents contemplated by the present invention are navelbene, CPT-11, anastrazole, letrazole, capecitabine, reloxafine, cyclophosphamide, ifosamide, and droloxafine.

The present invention also encompasses a pharmaceutical composition useful in the treatment of cancer, comprising the administration of a therapeutically effective amount of an IGF-1R/IR inhibitor, either alone or in combination with another agent, with or without pharmaceutically acceptable carriers or diluents. The compositions of the present invention may further comprise one or more pharmaceutically acceptable additional ingredient(s) such as alum, stabilizers, antimicrobial agents, buffers, coloring agents, flavoring agents, adjuvants, and the like. The IGF-1R/IR inhibitor, or analogs thereof, PDFGR-α inhibitor, or analogs thereof, or EGFR-inhibitors, or analogs thereof, antineoplastic agents, and compositions of the present invention may be administered orally or parenterally including the intravenous, intramuscular, intraperitoneal, subcutaneous, rectal and topical routes of administration.

For oral use, the antineoplastic agents, IGF-1R/IR inhibitor, or analogs thereof and compositions of this invention may be administered, for example, in the form of tablets or capsules, powders, dispersible granules, or cachets, or as aqueous solutions or suspensions. In the case of tablets for oral use, carriers which are commonly used include lactose, corn starch, magnesium carbonate, talc, and sugar, and lubricating agents such as magnesium stearate are commonly added. For oral administration in capsule form, useful carriers include lactose, corn starch, magnesium carbonate, talc, and sugar. When aqueous suspensions are used for oral administration, emulsifying and/or suspending agents are commonly added.

In addition, sweetening and/or flavoring agents may be added to the oral compositions. For intramuscular, intraperitoneal, subcutaneous and intravenous use, sterile solutions of the active ingredient(s) are usually employed, and the pH of the solutions should be suitably adjusted and buffered. For intravenous use, the total concentration of the solute(s) should be controlled in order to render the preparation isotonic.

For preparing suppositories according to the invention, a low melting wax such as a mixture of fatty acid glycerides or cocoa butter is first melted, and the active ingredient is dispersed homogeneously in the wax, for example by stirring. The molten homogeneous mixture is then poured into conveniently sized molds and allowed to cool and thereby solidify.

Liquid preparations include solutions, suspensions and emulsions. Such preparations are exemplified by water or water/propylene glycol solutions for parenteral injection. Liquid preparations may also include solutions for intranasal administration.

Aerosol preparations suitable for inhalation may include solutions and solids in powder form, which may be in combination with a pharmaceutically acceptable carrier, such as an inert compressed gas.

Also included are solid preparations which are intended for conversion, shortly before use, to liquid preparations for either oral or parenteral administration. Such liquid forms include solutions, suspensions and emulsions.

The IGF-1R/IR inhibitor, or analogs thereof, as well as anti-neoplastic agents, described herein may also be delivered transdermally. The transdermal compositions can take the form of creams, lotions, aerosols and/or emulsions and can be included in a transdermal patch of the matrix or reservoir type as are conventional in the art for this purpose.

The combinations of the present invention may also be used in conjunction with other well known therapies that are selected for their particular usefulness against the condition that is being treated.

If formulated as a fixed dose, the active ingredient(s) of the microtubulin-stabilizing agents, or combination compositions, of this invention are employed within the dosage ranges described below. Alternatively, the anti-CTLA4 agent, and IGF-1R/IR inhibitor, or analogs thereof may be administered separately in the dosage ranges described below. In a preferred embodiment of the present invention, the anti-CTLA4 agent is administered in the dosage range described below following or simultaneously with administration of the IGF-1R/IR inhibitor, or analogs thereof compound in the dosage range described below.

The following sets forth preferred therapeutic combinations and exemplary dosages for use in the methods of the present invention.

| | Dosage | |
|---|---|---|
| Therapeutic Combination | mg/m² (per dose) | |
| Compound of Formula I (BMS-754807) | 1-500 | mg/m² |
| Compound of Formula I (BMS-754807) | 1-500 | mg/m² |
| + PDGFR-α Inhibitor | 0.1-25 | mg/kg |
| Compound of Formula I (BMS-754807) | 1-500 | mg/m² |
| + EGFR Inhibitor | 0.1-25 | mg/kg |
| Anti-IGF-1R/IR Antibody | 1-500 | mg/m² |
| Anti-IGF-1R/IR Antibody | 1-500 | mg/m² |
| + PDGFR-α Inhibitor | 0.1-25 | mg/kg |
| Anti-IGF-1R/IR Antibody | 0.1-100 | mg/m² |
| + EGFR Inhibitor | 0.1-25 | mg/kg |

While this table provides exemplary dosage ranges of the IGF-1R/IR inhibitors and certain anticancer agents of the invention, when formulating the pharmaceutical compositions of the invention the clinician may utilize preferred dosages as warranted by the condition of the patient being treated. For example, the compound of Formula I may preferably be administered at about 4, 10, 20, 30, 50, 70, 100, 130, 160, or 200 mg/m² daily.

The anti-IGF-1R/IR antibody may preferably be administered at about 0.3-10 mg/kg, or the maximum tolerated dose. In an embodiment of the invention, a dosage of IGF-1R/IR antibody is administered about every three weeks. Alternatively, the IGF-1R/IR antibody may be administered by an escalating dosage regimen including administering a first dosage of IGF-1R/IR antibody at about 3 mg/kg, a second dosage of IGF-1R/IR antibody at about 5 mg/kg, and a third dosage of IGF-1R/IR antibody at about 9 mg/kg.

In another specific embodiment, the escalating dosage regimen includes administering a first dosage of IGF-1R/IR antibody at about 5 mg/kg and a second dosage of IGF-1R/IR antibody at about 9 mg/kg.

Further, the present invention provides an escalating dosage regimen, which includes administering an increasing dosage of IGF-1R/IR antibody about every six weeks.

In an aspect of the present invention, a stepwise escalating dosage regimen is provided, which includes administering a first IGF-1R/IR antibody dosage of about 3 mg/kg, a second IGF-1R/IR antibody dosage of about 3 mg/kg, a third IGF-1R/IR antibody dosage of about 5 mg/kg, a fourth IGF-1R/IR antibody dosage of about 5 mg/kg, and a fifth IGF-1R/IR antibody dosage of about 9 mg/kg. In another aspect of the present invention, a stepwise escalating dosage regimen is provided, which includes administering a first dosage of 5 mg/kg, a second dosage of 5 mg/kg, and a third dosage of 9 mg/kg.

The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired. Intermittent therapy (e.g., one week out of three weeks or three out of four weeks) may also be used.

In accordance with the diagnostic methods of the present invention, a treatment regimen may be assigned according to whether the patient is predicted to have a favorable or a less than favorable response. For those individuals predicted to have a favorable response, an ordinary IGF-1R/IR inhibitor dosing regimen may be administered. However, for those patients who are predicted to have a lower likelihood of achieving a favorable response (i.e., those individuals having BRAF^{V600E} or KRAs^{G13D} or PIK3CA mutations in exon 20, or those individuals having increased expression of IGFBP6, decreased expression of IR-A, or decreased IGF1R expression, for example), an increased dosage of an IGF-1R/IR inhibitor or an IGF-1R/IR inhibitor in combination with other therapy may be warranted. Such an increased level of a therapeutically-effective dose of an IGF-1R/IR inhibitor or an IGF-1R/IR inhibitor in combination with other therapy for an individual identified as being less likely to have a favorable response can be, for example, about 5%, 10%, 15%, 20%, 25%, 30%, 35%, 40%, 45%, 50%, 55%, 60%, 65%, 75%, 80%, 85%, 90%, or 95° A higher, or 1.5-, 2-, 2.5-, 3-, 3.5-, 4-, 4.5-, or even 5-fold higher than the prescribed or typical dose, as may be the case.

Alternatively, for those patients who are predicted to have a lower likelihood of achieving a favorable response (i.e., those individuals having elevated expression of AXL, EGFR, IGFBP, PDGFR-α, or those individuals having decreased expression of IGF-1R/IR), an increased frequency dosing regimen of an IGF-1R/IR inhibitor, and/or an IGF-1R/IR inhibitor in combination with other therapy may be warranted. Such an increased frequency dosing regimen of a therapeutically-effective dose of an IGF-1R/IR inhibitor and/or an IGF-1R/IR inhibitor in combination with other therapy for an individual identified as being less likely to have a favorable response can be, for example, about per once week, about once per 6 days, about once per 5 days, about once per 4 days, about once per 3 days, about once per 3 days, about once per 2 days, about once per day, about twice per day, about three per day, about four per day, or about 5%, 10%, 15%, 20%, 25%, 30%, 35%, 40%, 45%, 50%, 55%, 60%, 65%, 75%, 80%, 85%, 90%, or 95% higher, or 1.5-, 2-, 2.5-, 3-, 3.5-, 4-, 4.5-, or even 5-fold higher dosing frequency than the prescribed or typical dose, as may be the case.

In the instance where it may be desirable to administer a microtubulin stabilizing agent, such as paclitaxel or carboplatin, to the IGF-1R/IR treatment, or to the combination treatment of and IGF-1R/IR inhibitor with a PDGFR-α inhibitor and/or EGFR inhibitor, paclitaxel may be administered about 200 mg/m², Day 1 of a 21-day cycle via IV, whereas carboplatin may be administered about 6 mg/ml/min, Day 1 of a 21-day cycle via IV.

In the instance where it may be desirable to administer a HER2 inhibitor, such as HERCEPTIN®, to the IGF-1R/IR treatment, or to the combination treatment of and IGF-1R/IR inhibitor with a PDGFR-α inhibitor and/or EGFR inhibitor, HERCEPTIN® may be administered about 4 mg/kg Day 1 loading dose, 2 mg/kg once weekly via IV.

Certain cancers can be treated effectively with compounds of IGF-1R/IR inhibitor, PDGFR-α inhibitor, and/or EGFR inhibitor and a one or more anti-CTLA4 agents. Such triple and quadruple combinations can provide greater efficacy. When used in such triple and quadruple combinations the dosages set forth above can be utilized.

When employing the methods or compositions of the present invention, other agents used in the modulation of tumor growth or metastasis in a clinical setting, such as antiemetics, can also be administered as desired.

The present invention encompasses a method for the synergistic treatment of cancer comprising the administration of a synergistic combination of an IGF-1R/IR inhibitor and PDGFR-α inhibitor wherein said administration is performed simultaneously or sequentially. Thus, while a pharmaceutical formulation comprising an IGF-1R/IR inhibitor in combination with a PDGFR-α inhibitor may be advantageous for administering the combination for one particular treatment, prior administration of the PDGFR-α inhibitor may be advantageous in another treatment. It is also understood that the instant combination of IGF-1R/IR inhibitor and PDGFR-α inhibitor, may be used in conjunction with other methods of treating cancer (preferably cancerous tumors) including, but not limited to, radiation therapy and surgery. It is further understood that a cytostatic or quiescent agent, if any, may be administered sequentially or simultaneously with any or all of the other synergistic therapies.

The combinations of the instant invention may also be co-administered with other well known therapeutic agents that are selected for their particular usefulness against the condition that is being treated. Combinations of the instant invention may alternatively be used sequentially with known pharmaceutically acceptable agent(s) when a multiple combination formulation is inappropriate.

The chemotherapeutic agent(s) and/or radiation therapy can be administered according to therapeutic protocols well known in the art. It will be apparent to those skilled in the art that the administration of the chemotherapeutic agent(s) and/or radiation therapy can be varied depending on the disease being treated and the known effects of the chemotherapeutic agent(s) and/or radiation therapy on that disease. Also, in accordance with the knowledge of the skilled clinician, the therapeutic protocols (e.g., dosage amounts and times of administration) can be varied in view of the observed effects of the administered therapeutic agents on the patient, and in view of the observed responses of the disease to the administered therapeutic agents.

In the methods of this invention, a compound of Formula I or an IGF-1R/IR inhibitor is administered simultaneously or sequentially with a PDGFR-α inhibitor and/or an EGFR inhibitor. Thus, it is not necessary that the PDGFR-α inhibitor and/or an EGFR inhibitor and IGF-1R/IR inhibitor, be administered simultaneously or essentially simultaneously. The advantage of a simultaneous or essentially simultaneous administration is well within the determination of the skilled clinician.

Also, in general, the IGF-1R/IR inhibitor, do not have to be administered in the same pharmaceutical composition, and may, because of different physical and chemical characteristics, have to be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice an IGF-1R/IR inhibitor, PDGFR-α inhibitor, and/or EGFR inhibitor or analogs thereof will depend upon the diagnosis of the attending physicians and their judgment of the condition of the patient and the appropriate treatment protocol.

If the compound of Formula I or an anti-IGF-1R/IR antibody are not administered simultaneously or essentially simultaneously, then the initial order of administration of the compound of Formula I or IGF-1R/IR inhibitor, may be varied. Examples of different orders of administration are outlined elsewhere herein. The alternate administrations outlined herein may be repeated during a single treatment protocol. The determination of the order of administration, and the number of repetitions of administration of each therapeutic agent during a treatment protocol, is well within the knowledge of the skilled physician after evaluation of the disease being treated and the condition of the patient. The treatment is then continued with the administration of the compound of formula I or an IGF-1R/IR inhibitor or analogs thereof and optionally followed by administration of a cytostatic agent, if desired, until the treatment protocol is complete. Alternatively, the administration of the compound of Formula I or an IGF-1R/IR inhibitor or analogs thereof and optionally followed by administration of a cytostatic agent may be administered initially. The treatment is then continued with the administration of a cytostatic agent, such as for PDGFR-α inhibitor, and/or EGFR inhibitor, until the treatment protocol is complete.

Thus, in accordance with experience and knowledge, the practicing physician can modify each protocol for the administration of a component (therapeutic agent—i.e., compound of IGF-1R/IR inhibitor, or analogs thereof, anti-IGF-1R/IR antibody agent(s)) of the treatment according to the individual patient's needs, as the treatment proceeds.

The attending clinician, in judging whether treatment is effective at the dosage administered, will consider the general well-being of the patient as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

Thus, the present invention provides methods for the treatment of a variety of cancers, including, but not limited to, the following: carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyosarcoma, and osteosarcoma; other tumors including melanoma, xenoderma pigmentosum, keratoactanthoma, seminoma, thyroid follicular cancer, and teratocarcinoma; melanoma, unresectable stage III or IV malignant melanoma, squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, and head and neck cancer, gastric cancer, germ cell tumor, bone cancer, bone tumors, adult malignant fibrous histiocytoma of bone; childhood malignant fibrous histiocytoma of bone, sarcoma, pediatric sarcoma, sinonasal natural killer, neoplasms, plasma cell neoplasm; myelodysplastic syndromes; neuroblastoma; testicular germ cell tumor, intraocular melanoma, myelodysplastic syndromes; myelodysplastic/myeloproliferative diseases, synovial sarcoma, chronic myeloid leukemia, acute lymphoblastic leukemia, philadelphia chromosome positive acute lymphoblastic leukemia (Ph+ ALL), multiple myeloma, acute myelogenous leukemia, chronic lymphocytic leukemia, mastocytosis and any symptom associated with mastocytosis, and any metastasis thereof. In addition, disorders include urticaria pigmentosa, mastocytosises such as diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, mast cell leukemia, in addition to other cancers. Other cancers are also included within the scope of disorders including, but are not limited to, the following: carcinoma, including that of the bladder, urothelial carcinoma, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, teratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xenoderma pigmentosum, keratoactanthoma, seminoma, thyroid follicular cancer, teratocarcinoma, chemotherapy refractory non-seminomatous germ-cell tumors, and Kaposi's sarcoma, and any metastasis thereof.

Most preferably, the invention is used to treat accelerated or metastatic cancers of the breast and/or lung.

### Investigation

As both IGF-1R antibody and small molecular inhibitors are currently in clinical testing, it is critically important to understand the mechanisms of sensitivity to IGF-1R inhibitors because there may be only a subset of patients who respond to IGF-IR inhibitors. As a result, devising a strategy for rationally selecting patients most likely to derive clinical benefit could help clinical development of IGF1R/IR inhibitors, such as BMS-754807. This study focused on the identification of biomarkers that could be used to guide clinical development of IGF1R/IR inhibitors such as BMS-754807 in CRC.

The inventors conducted pre-clinical pharmacogenomic studies in a panel of colorectal cancer cell lines to identify candidate biomarkers that were significantly correlated with the sensitivity/resistance to BMS-754807. Several markers were identified through DNA copy number variation, mutational and gene expression analyses, IRS2 amplification, KRAS mutation, BRAF mutation, PIK3CA mutation, IR-A expression, IGF1R expression, and IGFBP6 expression to be correlated to the sensitivity to BMS-754807 in CRC cell lines.

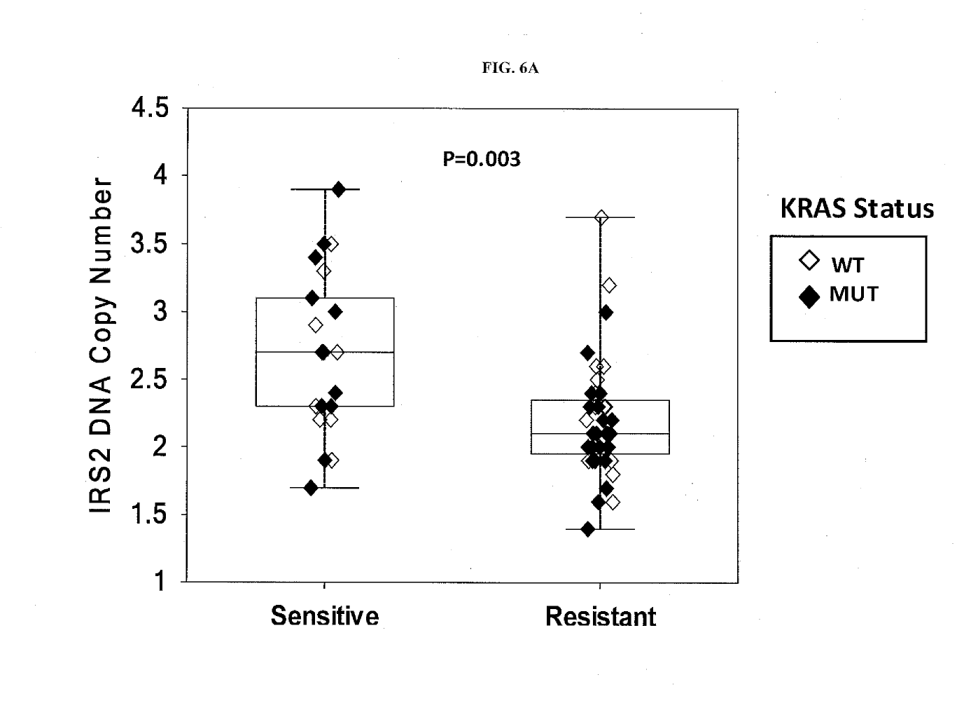

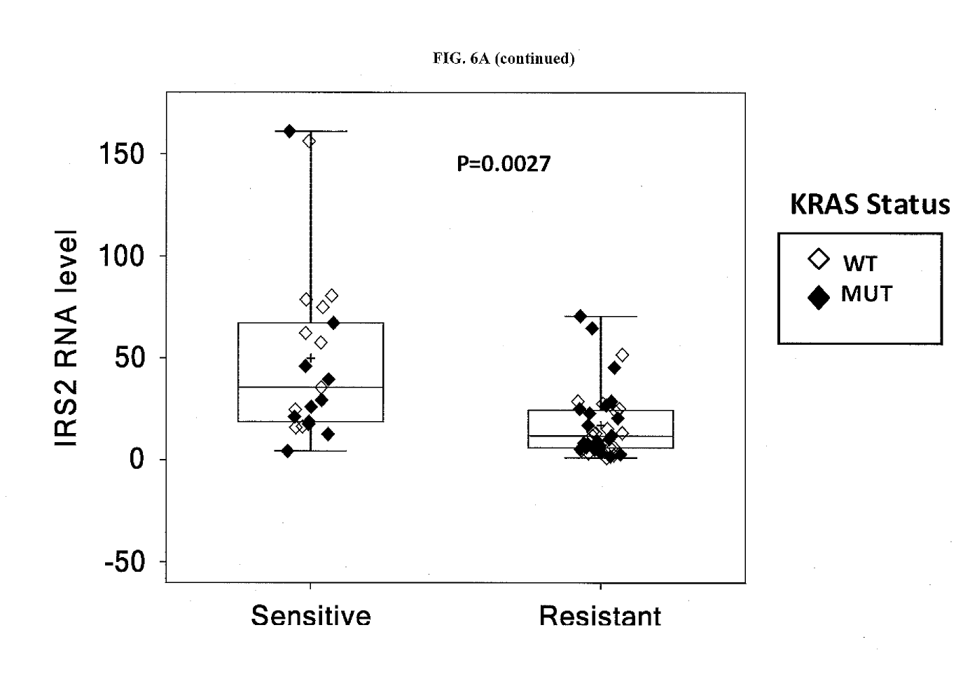

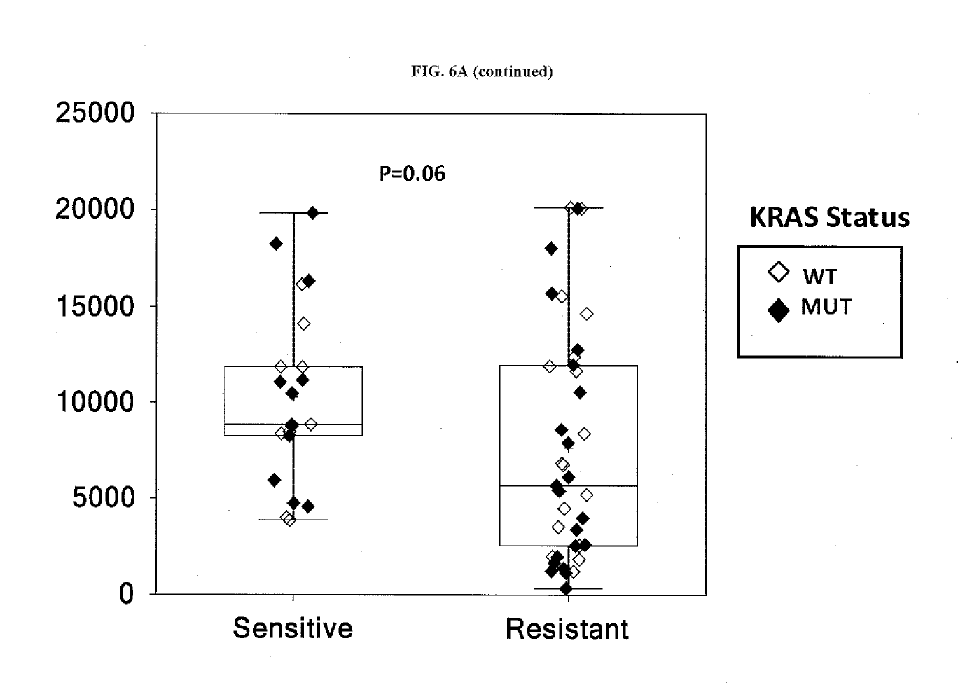

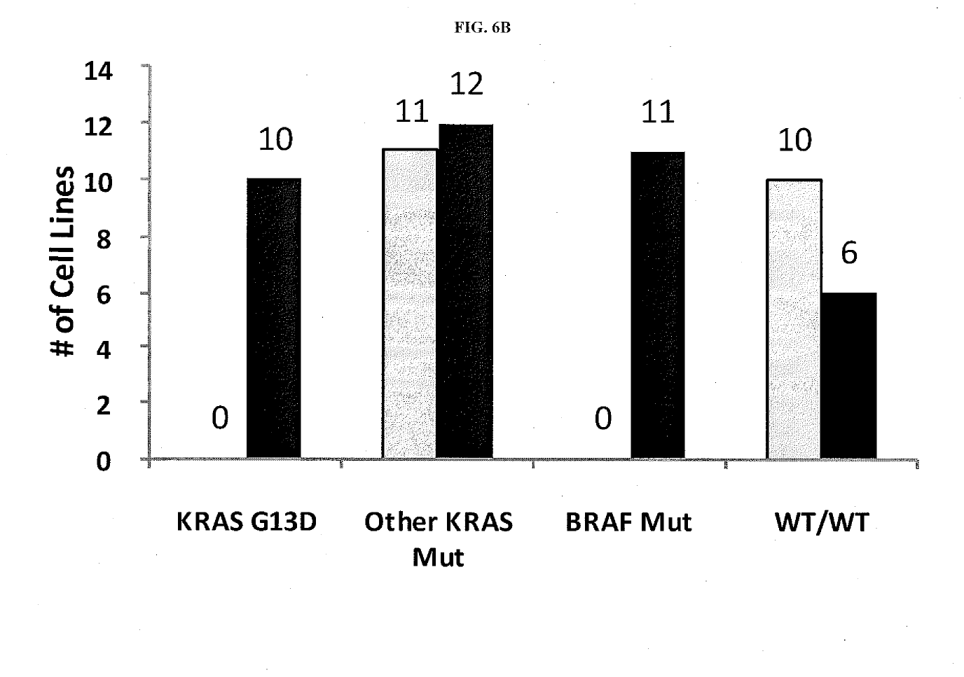

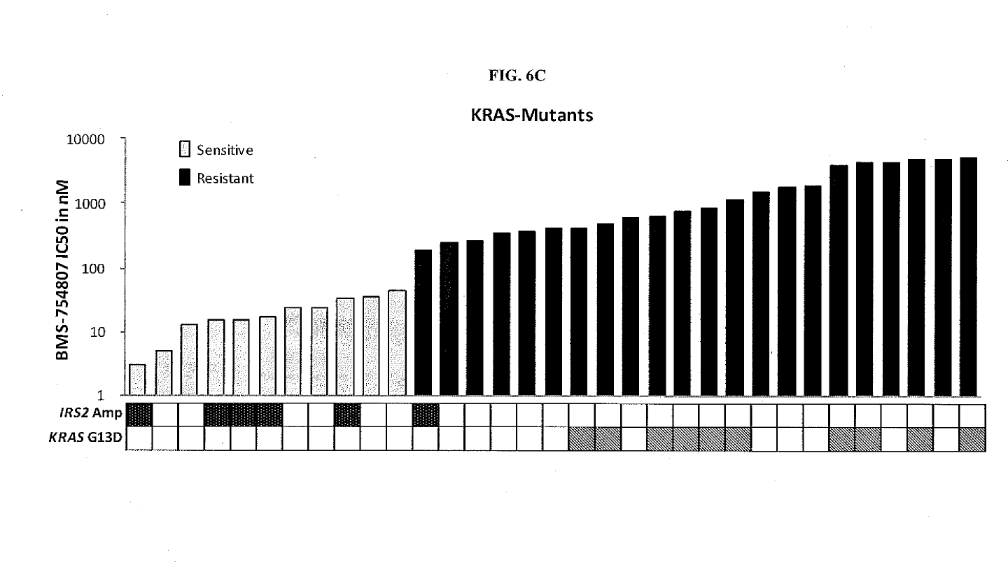

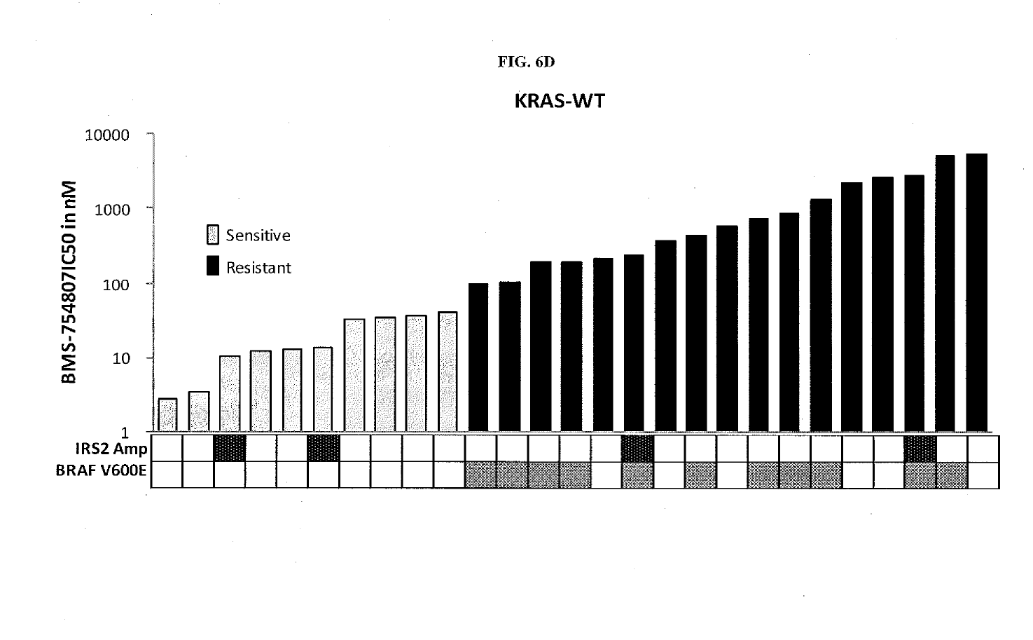

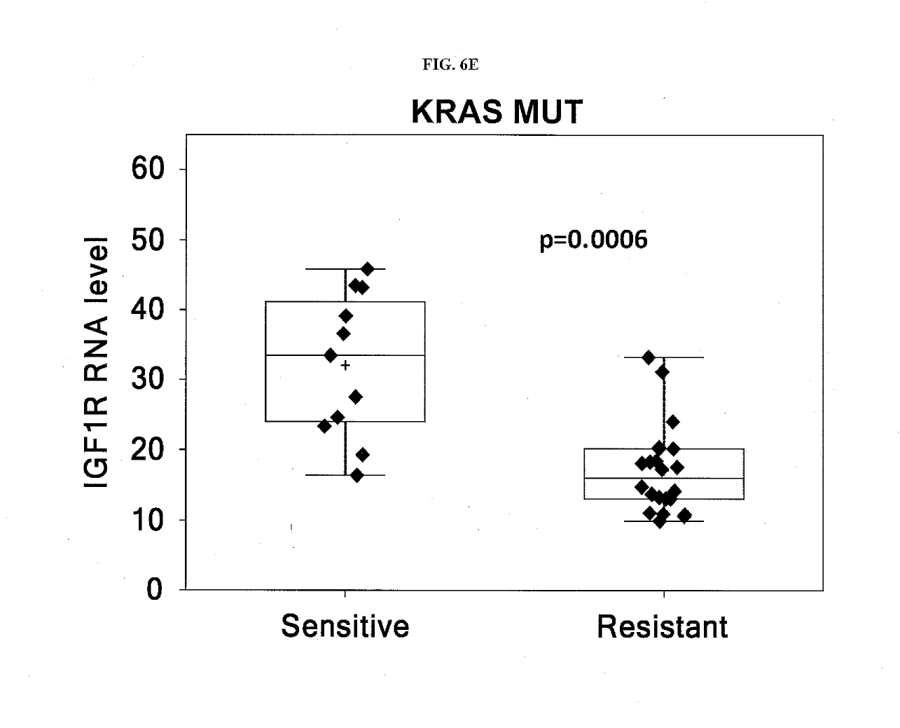

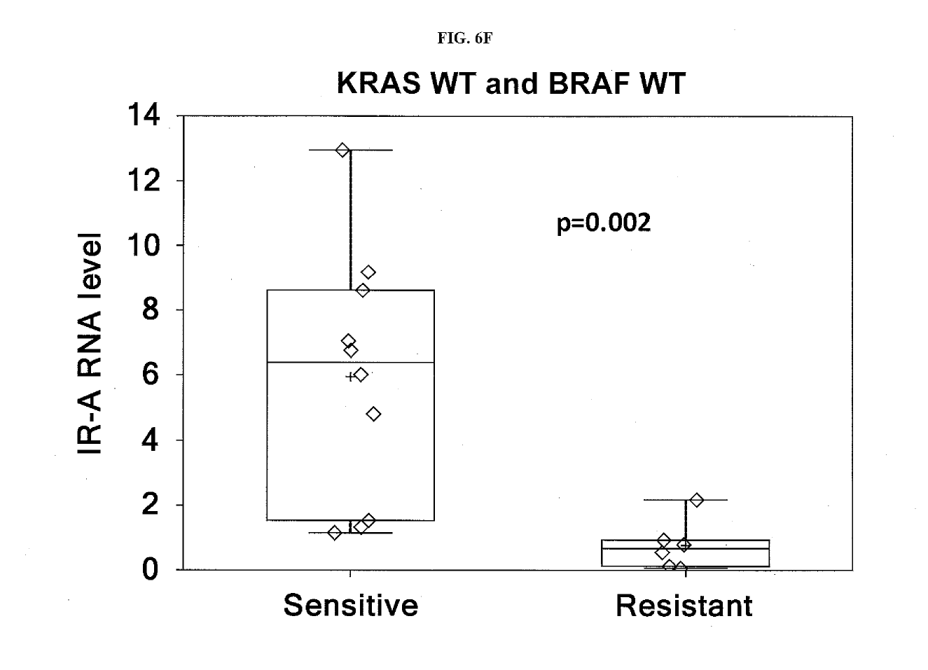

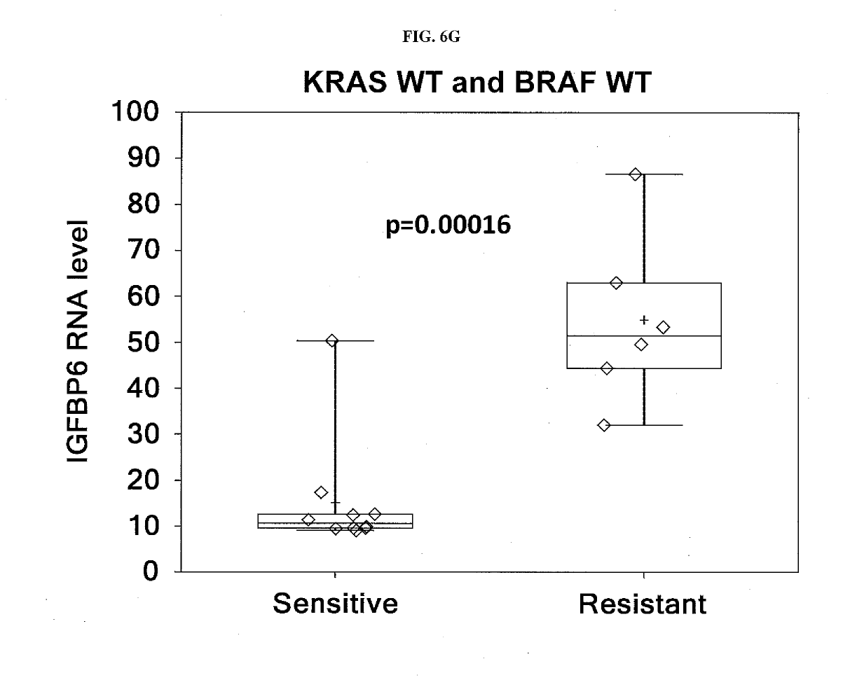

These candidate biomarkers are consistent with the biology of the targeted pathway: IRS2 is a cytoplasmic signaling molecule that mediates effects of insulin, IGF-1 by acting as a molecular adaptor between receptor tyrosine kinases and downstream effectors. It is postulated that the tumors with IRS2 amplification may indicate the IGF1R/IR pathway activation and tumor's dependence on this pathway for driving proliferation, therefore tumors are more responsive to IGF1R/IR targeting agents. In non-IRS2 amplified tumors with KRAS mutation, the MAKP/ERK pathway is constitutively activated leading to less dependence on IGF1R/IR pathway for proliferation, so less responsive to BMS-754807. On the other hand, higher level of IGFBPs may limit bioavailability of 1GF ligands, therefore cause less IGF1R/IR pathway activation. Further testing these biomarkers on whether they are necessary and/or sufficient for the differential sensitivity to agents targeting the IGF signaling pathway is needed to validate the hypotheses. Activation of IR signaling or increased expression of the IR-A isoform was observed in cancer cell lines when treated with a selective anti-IGF-1R antibody (13, 48) supporting the notion that activation of the IR-A/IGF2 autocrine loop represents a mechanism of resistance to IGF-1R antibody therapies. Our results demonstrate that KRAS/BRAF-WT cell lines with higher expression of IR-A were more sensitive to BMS-754807 than cells with lower IR-A RNA levels (FIG. 6F), supporting co-targeting IGF-1R and IR with a dual inhibitor such as BMS-754807, which may have enhanced efficacy against biomarker-selected tumors compared with an inhibitor, such as an IGF-1R mAb, that targets only IGF-1R. Lastly, activation of IGF-1R/IR pathway results in increased sensitivity to IGF-1R/IR TKI inhibition, leading to decreased downstream PI3K/AKT and RAS/RAF/ERK signaling and consequently decreased in cell proliferation.

### Biomarkers and Biomarker Sets

The invention includes individual biomarkers and biomarker sets having both diagnostic and prognostic value in proliferative disease areas in which IGF-1R/IR is of importance, e.g., in cancers or tumors, or in disease states in which cell signaling and/or cellular proliferation controls are abnormal or aberrant. The biomarker sets comprise a plurality of biomarkers that highly correlate with resistance or sensitivity to one or more IGF-1R/IR agents.

The biomarkers and biomarker sets of the invention enable one to predict or reasonably foretell the likely effect of one or more IGF-1R/IR agents in different biological systems or for cellular responses merely based upon whether one or more of the biomarkers of the present invention are amplified, overexpressed, or under expression, relative to normal or a predetermined level or reference level of expression. The biomarkers and biomarker sets can be used in in vitro assays of cellular proliferation by sample cells to predict in vivo outcome. In accordance with the invention, the various biomarkers and biomarker sets described herein, or the combination of these biomarker sets with other biomarkers or markers, can be used, for example, to predict and monitor how patients with cancer might respond to therapeutic intervention with one or more IGF-1R/IR inhibitors.

Measuring the level of expression of a biomarker and biomarker set provides a useful tool for screening one or more tumor samples before treatment of a patient with the IGF-1R/IR inhibitor. The screening allows a prediction of whether the cells of a tumor sample will respond favorably to a IGF-1R/IR inhibitor, based on the presence or absence of amplification, over-expression, or underexpression—such a prediction provides a reasoned assessment as to whether or not the tumor, and hence a patient harboring the tumor, may or may not have a favorable response to treatment with a IGF-1R/IR inhibitors.

A difference in the level of the biomarker that is sufficient to indicate whether a patient may or may not have a favorable therapeutic response to the method of treating cancer can be readily determined by one of skill in the art. The increase or decrease in the level of the biomarker can be correlated to determine whether the difference is sufficient to identify a mammal that will respond therapeutically. The difference in the level of the biomarker that is sufficient can, in one aspect, be predetermined prior to determining whether the patient will respond therapeutically to the treatment. In one aspect, the difference in the level of the biomarker is a difference in the mRNA level (measured, for example, by RT-PCR or a microarray), such as at least about a two-fold difference, at least about a three-fold difference, or at least about a four-fold difference in the level of expression, or more. In another aspect, the difference in the level of the biomarker is determined at the protein level by mass spectral methods or by IHC. In another aspect, the difference in the level of the biomarker is determined by FISH assay or qPCR assay, among other assays known in the art.

The biomarkers also serve as targets for the development of therapies for disease treatment. Such targets may be particularly applicable to treatment of cancer, such as, for example, colon, breast and/or lung cancer.

Indeed, because these biomarkers are differentially expressed in sensitive and resistant cells, their expression patterns are correlated with relative intrinsic sensitivity of cells to treatment with IGF-1R/IR inhibitors. Accordingly, the biomarkers over expressed in resistant cells may serve as targets for the development of new therapies for the tumors which are resistant to IGF-1R/IR inhibitors. The level of biomarker protein and/or mRNA can be determined using methods well known to those skilled in the art. For example, quantification of protein can be carried out using methods such as ELISA, 2-dimensional SDS PAGE, Western blot, immunoprecipitation, immunohistochemistry, fluorescence activated cell sorting (FACS), or flow cytometry. Quantification of mRNA can be carried out using methods such as PCR, array hybridization, Northern blot, in-situ hybridization, dot-blot, TAQMAN®, or RNAse protection assay.

The present invention encompasses the use of any one or more of the following as a biomarker, either alone or in conjunction with each other, for use in predicting IGF-1R/IR inhibitors response: IRS2 copy number, KRAS mutation status, BRAF mutation status, PIK3CA mutation status, IR-A expression levels, IGF1R expression levels, and IGFBP6 expression levels.

The present invention also encompasses any combination of the aforementioned biomarkers, including, but not limited to: IRS2 copy number; KRAS mutation status; BRAF mutation status; PIK3CA mutation status; IR-A expression level; IGF1R expression level; IGFBP6 expression level; IRS2 copy number and KRAS mutation status; IRS2 copy number and BRAF mutation status; IRS2 copy number and PIK3CA mutation status; IRS2 copy number and IR-A expression level; IRS2 copy number and IGF1R expression level; IRS2 copy number and IGFBP6; KRAS mutation status and BRAF mutation status; KRAS mutation status and PIK3CA mutation status; KRAS mutation status and IR-A expression level; KRAS mutation status and IGF1R expression level; KRAS mutation status and IGFBP6 expression level; BRAF mutation status and PIK3CA mutation status; BRAF mutation status and IR-A expression level; BRAF mutation status and IGF1R expression level; BRAF mutation status and IGFBP6 expression level; PIK3CA mutation status and IR-A expression level; PIK3CA mutation status and IGF1R expression level; PIK3CA mutation status and IGFBP6 expression level; IR-A expression level and IGF1R expression level; IR-A expression level and IGFBP6 expression level; or any combination thereof.

Identification of biomarkers that provide rapid and accessible readouts of efficacy, drug exposure, or clinical response is increasingly important in the clinical development of drug candidates. Embodiments of the invention include measuring gene copy number in a sample to determine whether said sample contains increased, normal or decreased copy number of IRS2. Embodiments of the invention include determining whether a patient sample contains one or more KRAS mutants. Embodiments of the invention include determining whether a patient sample contains one or more BRAF mutants. Embodiments of the invention include determining whether a patient sample contains one or more PIK3CA mutants. Embodiments of the invention include measuring changes in the levels of mRNA and/or protein in a sample to determine whether said sample contains increased or decreased expression of IGFBP6. Embodiments of the invention include measuring changes in the levels of mRNA and/or protein in a sample to determine whether said sample contains increased or decreased expression of IR-A. Embodiments of the invention include measuring changes in the levels of mRNA and/or protein in a sample to determine whether said sample contains increased or decreased expression of IGF1R. In one aspect, said samples serve as surrogate tissue for biomarker analysis. These biomarkers can be employed for predicting and monitoring response to one or more IGF-1R/IR inhibitors. In one aspect, the biomarkers of the invention are one or more of the following: IRS2 copy number, KRAS mutation status, and IGFBP6 expression levels, including both polynucleotide and polypeptide sequences. In another aspect, the biomarkers of the invention are nucleotide sequences that, due to the degeneracy of the genetic code, encodes for a polypeptide sequence provided in the sequence listing.

The biomarkers serve as useful molecular tools for predicting and monitoring response to IGF-1R/IR inhibitors.

Methods of detecting or measuring the level of expression and/or amplication of any given marker described herein may be performed using methods well known in the art, which include, but are not limited to sequencing, PCR; RT-PCR; FISH; IHC; immunodetection methods; immunoprecipitation; Western Blots; ELISA; radioimmunoassays; FACS; HPLC; surface plasmon resonance, and optical spectroscopy; and mass spectrometry, among others. For example, amplification of IRS2 can be determined by FISH or qPCR assays. Quantification of IGFBP6 level can be carried out using methods such as qRT-PCR, array hybridization, Northern blot, in-situ hybridization, dot-blot, TAQMAN®, or RNAse protection assay. IHC. Presence of a KRAS mutation can be detected by any sequencing method, including dideoxy sequencing, pyrosequencing, PYROMARK® KRAS assays, allele-specific PCR assays.

The biomarkers of the invention may be quantified using any immunospecific binding method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds., Current Protocols in Molecular Biology, Vol. 1, John Wiley & Sons, Inc., New York (1994), which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% TRASYLOL®) supplemented with protein phosphatase and/or protease inhibitors (e.g., EDTA, PMSF, aprotinin, sodium vanadate), adding the antibody of interest (i.e., one directed to a biomarker of the present invention) to the cell lysate, incubating for a period of time (e.g., 1-4 hours) at 4° C., adding protein A and/or protein G SEPHAROSE® beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with SEPHAROSE® beads). For further discussion regarding immunoprecipitation protocols see, e.g., Ausubel et al., eds., Current Protocols in Molecular Biology, Vol. 1, p. 10.16.1, John Wiley & Sons, Inc., New York (1994).

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds., Current Protocols in Molecular Biology, Vol. 1, p. 10.8.1, John Wiley & Sons, Inc., New York (1994).

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al., eds., Current Protocols in Molecular Biology, Vol. 1, p. 11.2.1, John Wiley & Sons, Inc., New York (1994).

Alternatively, identifying the relative quantitation of the biomarker polypeptide(s) may be performed using tandem mass spectrometry; or single or multi dimensional high performance liquid chromatography coupled to tandem mass spectrometry. The method takes into account the fact that an increased number of fragments of an identified protein isolated using single or multi dimensional high performance liquid chromatography coupled to tandem mass spectrometry directly correlates with the level of the protein present in the sample. Such methods are well known to those skilled in the art and described in numerous publications, for example, Link, A. J., ed., 2-D Proteome Analysis Protocols, Humana Press (1999), ISBN: 0896035247; Chapman, J. R., ed., Mass Spectrometry of Proteins and Peptides, Humana Press (2000), ISBN: 089603609X.

As used herein the terms “modulate” or “modulates” or “modulators” refer to an increase or decrease in the amount, quality or effect of a particular activity, or the level of DNA, RNA, or protein detected in a sample.

In order to facilitate a further understanding of the invention, the following examples are presented primarily for the purpose of illustrating more specific details thereof. The scope of the invention should not be deemed limited by the examples, but to encompass the entire subject matter defined by the claims.

### REFERENCES

1. Jemal, A. et al., “Cancer Statistics 2009”, CA Cancer J. Clin., 59:225-249 (2010).
2. Samani, A. A. et al., “The role of the IGF system in cancer growth and metastasis: overview and recent insights”, Endocr. Rev., 28:20-47 (2007).
3. Frasca, F. et al., “The role of insulin receptors and IGF-I receptors in cancer and other diseases”, Arch. Physiol. Biochem., 114(1):23-37 (February 2008).
4. Donovan, E. A. et al., “Role of insulin-like growth factor-1R system in colorectal carcinogenesis”, Crit. Rev. Oncol. Hematol., 66:91-98 (2008).
5. Ewing, G. P. et al., “The insulin-like growth factor signaling pathway as a target for treatment of colorectal carcinoma”, Clin. Colorectal Cancer, 9(4):219-223 (October 2010).
6. Gao, J. et al., “Targeting the insulin-like growth factor axis for the development of novel therapeutics in oncology”, Cancer Res., 72(1):3-12 (2012).
7. Gualberto, A. et al., “Emerging role of insulin-like growth factor receptor inhibitors in oncology: early clinical trial results and future directions”, Oncogene, 28:3009-3021 (2009).
8. Weroha, S. J. et al., “IGF-1 receptor inhibitors in clinical trials-Early lessons”, J. Mamm. Gland. Biol. Neoplasia, 13:471-483 (2008).
9. Pollak, M., “The insulin and insulin-like growth factor receptor family in neoplasia: an update”, Nat. Rev. Cancer, 12(3):159-169 (Feb. 16, 2012).
10. Belfiore, A. et al., “Insulin receptor isoforms and insulin receptor/insulin-like growth factor receptor hybrids in physiology and disease”, Endocr. Rev., 30(6):586-623 (October 2009).
11. Zhang, H. et al., “Down-regulation of type I insulin-like growth factor receptor increases sensitivity of breast cancer cells to insulin”, Cancer Res., 67:391-397 (2007).
12. Ulanet, D. B. et al., “Insulin receptor functionally enhances multistage tumor progression and conveys intrinsic resistance to IGF-1R targeted therapy”, Proc. Natl. Acad. Sci. USA, 107:10791-10798 (2010).
13. Buck, E. et al., “Compensatory Insulin Receptor (IR) Activation on Inhibition of Insulin-Like Growth Factor-1 Receptor (IGF-1R): Rationale for Cotargeting IGF-1R and IR in Cancer”, Mol. Cancer Ther., 9:2652-2664 (2010).
14. Brierley, G. V. et al., “Silencing of the Insulin Receptor Isoform A Favors Formation of Type 1 Insulin-Like Growth Factor Receptor (IGF-IR) Homodimers and Enhances Ligand-Induced IGF-IR Activation and Viability of Human Colon Carcinoma Cells”, Endocrinology, 151:1418-1427 (2010).
15. Huang, F. et al., “The mechanisms of differential sensitivity to an insulin-like growth factor-1 receptor inhibitor (BMS-536924) and rationale for combining with EGFR/HER2 inhibitors”, Cancer Res., 69:161-170 (2009).
16. Litzenburger, B. C. et al., “High IGF-IR activity in triple-negative breast cancer cell lines correlates with sensitivity to IGF-IR inhibitor BMS-754807 in this subtype of human breast cancer”, Cancer Res., 69:1132 (2009).
17. Pitts, T. M. et al., “Development of an integrated genomic classifier for a novel agent in colorectal cancer: approach to individualized therapy in early development”, Clin. Cancer Res., 16:3193-3204 (2010).
18. Gualberto, A. et al., “Pre-treatment levels of circulating free IGF-1 identify NSCLC patients who derive clinical benefit from figitumumab”, Br. J. Cancer, 104:68-74 (2011).
19. Tognon, C. E. et al., “Targeting the insulin-like growth factor 1 receptor (IGF1R) signaling pathway for cancer therapy”, Expert Opin. Ther. Targets, 16(1):33-48 (January 2012).
20. Yee, D., “Insulin-like Growth Factor Receptor Inhibitors: Baby or the Bathwater?”, J. Natl. Cancer Inst., 104(13):975-981 (Jul. 3, 2012).
21. Carboni, J. C. et al., “BMS-754807, a small molecule inhibitor of insulin-like growth factor-1R/IR”, Mol. Cancer Ther., 8:3341-3349 (2009).
22. Huang, F. et al., “Identification of Candidate Molecular Markers Predicting Sensitivity in Solid Tumors to Dasatinib: Rationale for Patient Selection”, Cancer Res., 67(5):2266-2238 (2007).
23. Forbes, S. A. et al., “COSMIC (the Catalogue of Somatic Mutations in Cancer): a resource to investigate acquired mutations in human cancer”, Nucleic Acids Res., 38 (Database issue):D652-D657 (2010).
24. Bengtsson, H. et al., “A single-array preprocessing method for estimating full-resolution raw copy numbers from all Affymetrix genotyping arrays including GenomeWideSNP 5 & 6”, Bioinformatics, 25:2149-2156 (2009).
25. Olshen, A. B. et al., “Circular binary segmentation for the analysis of array-based DNA copy number data”, Biostatistics, 5:557-572 (2004).
26. Clemens, P. L. et al., “BMS-754807, an oral dual IGF-1R/IR inhibitor: first-in-human single-dose study of safety, tolerability, pharmacokinetics, and pharmacodynamics in healthy subjects”, Mol. Cancer Ther., 8(12 Suppl):A101 (2009).
27. Desai, J. et al., “Phase I dose-escalation study of daily BMS-754807, an oral, dual IGF-1R/insulin receptor (IR) inhibitor in subjects with solid tumors”, J. Clin. Oncol., 28(15 Suppl, May 20), ASCO Meeting Abstracts, No. 3104 (Jun. 14, 2010).
28. Reynolds, J. M. et al., “Insulin-like growth factor 1 receptor mutants are inhibited by figitumumab (CP-751, 871) in vitro”, Mol. Cancer Ther., 8(12 Suppl):B119 (2009).
29. Reid, J. F. et al., “Integrative approach for prioritizing cancer genes in sporadic colon cancer”, Genes Chromosomes Cancer, 48(11):953-962 (November 2009).
30. Shoemaker, R. H. et al., “Development of human tumor cell line panels for use in disease-oriented drug screening”, Prog. Clin. Biol. Res., 276:265-286 (1988).
31. Weinstein, J. N. et al., “An information-intensive approach to the molecular pharmacology of cancer”, Science, 275:343-349 (1997).
32. McDermott, U. et al., “Identification of genotype-correlated sensitivity to selective kinase inhibitors by using high-throughput tumor cell line profiling”, Proc. Natl. Acad. Sci. USA, 104:19936-19941 (2007).
33. Kwak, E. L. et al., “Anaplastic lymphoma kinase inhibition in non-small-cell lung cancer”, N. Engl. J. Med., 363:1693-1703 (2010).
34. Chapman, P. B. et al., “Improved survival with vemurafenib in melanoma with BRAF V600E mutation”, N. Engl. J. Med., 364:2507-2516 (2011).
35. Barretina, J. et al., “The Cancer Cell Line Encyclopedia enables predictive modelling of anticancer drug sensitivity”, Nature, 483(7391):603-607 (Mar. 28, 2012).
36. Garnett, M. J. et al., “Systematic identification of genomic markers of drug sensitivity in cancer cells”, Nature, 483(7391):570-575 (Mar. 28, 2012).
37. Weinstein, J. N., “Drug discovery: Cell lines battle cancer”, Nature, 483(7391):544-545 (Mar. 29, 2012).
38. Bohanes, P. et al., “Predictive Molecular Classifiers in Colorectal Cancer”, Semin. Oncol., 38:576-587 (2011).
39. De Roock, W. et al., “KRAS, B-RAF, PIK3CA, and PTEN mutations: implications for targeted therapies in metastatic colorectal cancer”, Lancet Oncol., 12:594-603 (2011).
40. ERBITUX® (cetuximab) [package insert]. Branchburg, N.J. and Princeton, N.J.: ImClone LLC, a wholly-owned subsidiary of Eli Lilly and Company, and Bristol-Myers Squibb Company (January 2012).
41. De Roock, W. et al., “Association of KRAS p.G13D mutation with outcome in patients with chemotherapy-refractory metastatic colorectal cancer treated with cetuximab”, JAMA, 304:1812-1820 (2010).
42. Guerrero, S. et al., “K-ras codon 12 mutation induces higher level of resistance to apoptosis and predisposition to anchorage-independent growth than codon 13 mutation or proto-oncogene overexpression”, Cancer Res., 60:6750-6756 (2000).
43. van der Veeken, J. et al., “Crosstalk between epidermal growth factor receptor- and insulin-like growth factor-1 receptor signaling: implications for cancer therapy”, Curr. Cancer Drug Targets, 9:748-760 (2009).
44. Ebi, H. et al., “Receptor tyrosine kinases exert dominant control over PI3K signaling in human KRAS mutant colorectal cancers”, J. Clin. Invest., 121(11):4311-4321 (November 2011).
45. Samuels, Y. et al., “High frequency of mutations of the PIK3CA gene in human cancers”, Science, 304:554 (2004).
46. Nosho, K. et al., “PIK3CA mutation in colorectal cancer: relationship with genetic and epigenetic alterations”, Neoplasia, 10:534-541 (2008).
47. Zhao, L. et al., “Hot-spot mutations in p110alpha of phosphatidylinositol 3-kinase (pI3K): differential interactions with the regulatory subunit p85 and with RAS”, Cell Cycle, 9:596-600 (2010).
48. Haluska, P. et al., “Complete IGF Signaling Blockade by the Dual-Kinase Inhibitor, BMS-754807, Is Sufficient To Overcome Tamoxifen and Letrozole Resistance In Vitro and In Vivo”, Cancer Res., 69(24 Suppl):Abstract No. 402 (2009).

### EXAMPLES

### Materials and Methods

Cell lines and in vitro cytotoxicity assay. Cells were maintained at 37° C. under standard cell culture conditions. Cells were plated at an optimized density for each cell line per well in 96-well microtiter FALCON® plates, incubated overnight, and then exposed to a serial dilution of drug. After 72 hours incubation with drug at 37° C., cytotoxicity testing was evaluated using MTS assay to determine the sensitivity of cell lines to BMS-754807. The results were expressed as an IC₅₀, which is the drug concentration required to inhibit cell proliferation to 50% of that of untreated control cells. The mean IC₅₀ and standard deviation (SD) from multiple tests for each cell line were calculated.

Gene expression profiles. Total RNA was isolated from the cultured cells at about 70% confluence using the RNeasy kits from QIAGEN® (Valencia, Calif.). 1 μg of total RNA was used to prepare biotinylated probe and hybridized on Affymetrix HT-HG-133-plus PM arrays. The sample preparation and processing procedure were done as described in the Affymetrix GENECHIP® Expression Analysis Manual (Affymetrix, Inc.). The gene expression raw data were normalized by the Robust Multichip Average (RMA) method and log 2 transformed. To identify genes whose expression level significantly correlation with the drug sensitivity for the compounds, two separate statistic analyses were performed. First, a two-sample t-test between the resistant and sensitive cell lines (based on a threshold IC₅₀ cutoff of 50 nM) was performed. Second, Pearson correlation between the normalized expression level of each gene/protein and the log 2 (IC₅₀) values of the cell line panel was calculated to identify genes correlated with the drug sensitivity (IC₅₀).

Gene copy analysis. DNA was isolated from 5×106 cells using the DNeasy Blood and Tissue kit from QIAGEN® (Valencia, Calif.). Two aliquots of 250 ng genomic DNA per sample were digested by restriction enzymes NspI and StyI, respectively. The resulting products were ligated to the corresponding adaptors and PCR amplified. The labeled PCR products were hybridized to the Human SNP 6.0 array according to the Affymetrix recommendations. The Cel files were processed using an aroma.affymetrix package in the R-project. Segmentation of normalized raw copy number data was performed with the CBS algorithm implemented in the aroma the affymetrix package. Copy number gain (or loss) of a gene was obtained by using the maximum (or minimum) of segmented copy number values within the genomic region of the gene.

Method for Quantifying IGFBP6 Expression Levels using qPCR. Primer/probe mix was obtained from an inventoried IGFBP6 TAQMAN® Gene Expression assay from Applied Biosystems-Life Technologies, Cat# Hs00181853_m1. 20 ng of template cDNA samples were plated in triplicate in a 96 well plate along with a negative control (no-template) and standard curve CDNA. A Master mix was made using 1× TAQMAN® Gene Expression Master mix, 100 nM IGFBP6 primer/probe mix and DEPC H₂O to volume, and was added to the 8 ul template for a total volume of 50 ul, according to manufacturer protocol for the MICROAMP® Optical 96-Well Reaction Plate. QPCR was performed on the Applied Biosystems ABI 7900 real-time quantitative PCR instrument using Absolute Quantitation and the default cycling conditions. A corresponding b-ACTIN® plate was run using the same procedure as above with an inventoried Human ACTB (beta actin) Endogenous Control (VIC/MGB Probe, Primer Limited) TAQMAN® Gene Expression assay from Applied Biosystems-Life Technologies, Cat#4326315E. An average of the three C_{T} values from the IGFBP6 plate was taken and was compared and normalized using the standard curve; an average of the three C_{T} values from the b-ACTIN® plate was also taken and was compared and normalized using the standard curve for that plate, and the relative quantitative values generated from IGFBP6 plate was normalized to b-actin.

### Example 1

### Method of Assessing the Responsiveness of a Panel of CRC Cell Lines to the IGF-1R/IR Inhibitor BMS-754807

To evaluate the sensitivity of CRC cell lines to BMS-754807, a preliminary panel of 45 CRC cell lines was exposed to increasing concentrations of BMS-754807 and assessed for proliferation using a MTS assay. A broad range of sensitivity of the CRC cell lines was observed for BMS-754807. For categorization, a sensitive cell line was classified as one with an IC₅₀ of ≦50 nmol/L, whereas resistant cell lines had IC₅₀ values of >50 nmol/L; 15 cell lines classified as sensitive and remaining 30 cell lines classified as Intermediate or resistant) as indicated in Table 1.

TABLE 1
The status of KRAS, BRAF and PI3K gene mutations and
BMS-754807 sensitivity in a panel of CRC cell lines.

| | Sensitivity to | KRAS | BRAF | PIK3CA |
|---|---|---|---|---|
| Cell Line | BMS-754807 | Status * | Status ** | Status |
| SK-CO-1 | Sensitive | MUT | WT | WT |
| H508 | Sensitive | WT | MUT | MUT |
| DIFI | Sensitive | WT | ND | ND |
| SW48 | Sensitive | WT | WT | MUT |
| SNUC1 | Sensitive | WT | WT | WT |
| LS513 | Sensitive | MUT | WT | WT |
| SW1463 | Sensitive | MUT | WT | WT |
| SW948 | Sensitive | MUT | WT | MUT |
| SW1116 | Sensitive | MUT | WT | WT |
| KM12C | Sensitive | WT | WT | ND |
| SW403 | Sensitive | MUT | WT | WT |
| COLO320HSR | Sensitive | WT | WT | WT |
| KM12SM | Sensitive | WT | ND | ND |
| COLO320DM | Sensitive | WT | ND | ND |
| LS1034 | Sensitive | MUT | WT | WT |
| LS411N | Intermediate | WT | MUT | WT |
| SW1417 | Intermediate | WT | MUT | WT |
| CX-1 | Intermediate | WT | ND | ND |
| WIDR | Intermediate | WT | ND | ND |
| NCI-H716 | Intermediate | WT | WT | WT |
| COLO205 | Intermediate | WT | MUT | WT |
| LS174T | Intermediate | MUT | WT | MUT |
| GEO | Intermediate | MUT | WT | WT |
| HCT116SS42 | Intermediate | MUT | ND | ND |
| HT29 | Intermediate | WT | MUT | MUT |
| HCT116 | Resistant | MUT | WT | MUT |
| LS180 | Resistant | MUT | WT | MUT |
| DLD-1 | Resistant | MUT | WT | MUT |
| RKO-RM13 | Resistant | WT | ND | ND |
| HCT15 | Resistant | MUT | WT | MUT |
| RKO PM | Resistant | MUT | ND | ND |
| RKO | Resistant | WT | WT | MUT |
| NCI-H747 | Resistant | MUT | WT | WT |
| SNUC2B | Resistant | MUT | WT | WT |
| SW620 | Resistant | MUT | WT | WT |
| SW837 | Resistant | MUT | WT | WT |
| CCD18CO | Resistant | WT | ND | ND |
| COLO201 | Resistant | WT | MUT | WT |
| LOVO | Resistant | MUT | WT | WT |
| MIP | Resistant | MUT | ND | ND |
| LS123 | Resistant | MUT | WT | WT |
| HCT8 | Resistant | MUT | WT | MUT |
| SW480 | Resistant | MUT | WT | WT |
| T84 | Resistant | MUT | WT | MUT |
| CACO-2 | Resistant | WT | MUT | ND |

* Mutation test on KRAS was done for codons 12, 13, 61 and 146
** Mutation test on BRAF was done for codon 600
WT: wild type
MUT: mutation
ND: No Data

### Example 2

### Methods of Assessing Correlation Between KRAS/BRAF/PI3K Gene Mutation Status and Sensitivity to IGF-1R/IR Inhibition

As IGF signaling affects the Ras/Raf/MEK/MAPK and PI3K/AKT pathways, the present inventors characterized mutational status for KRAS for all 45 cell lines, BRAF and PI3K genes in the CRC cell lines and looked correlation between mutational status and BMS-754807 sensitivity (Table 1). There was a trend toward KRAS mutated tumors being more resistant, especially in the most resistant lines with 75% (15 out 20) cell lines with IC₅₀>500 nM having KRAS mutation. There was no obvious correlation between either BRAF, or PI3K mutation status and responsiveness to BMS-754807 from the available data.

### Example 3

### Methods of Assessing the Correlation Between Chromosome 13 Amplification and IGF-1R/IR Inhibitor Sensitivity

DNA copy number analysis was done using Affymetrix SNP 6 arrays for 43 CRC lines and Pearson correlation between the copy number for each gene and the IC₅₀ (log 10) value of BMS-754807 was computed. Amplification of chromosome 13 was found in a subset of cell lines and amplification in a region between 13q32-q34 containing 59 genes (Table 2) showed significant correlation (p<0.00005 and at least one sample >3 copy or <1 copy) to the IC₅₀ (log 10) values across the cell line panel (FIG. 1). Chromosome 13 amplification is not a cell culture artifact because it was not only seen in CRC cell lines, but also observed in primary colon cancers. IRS2, a gene encodes the insulin receptor substrate 2, and several other genes (RAB20, RASA3, RAP2A, RASL11A, ARHGEF7) related to RAS pathway function are in this region and the higher copy number of these genes were observed in cell lines that were more sensitive to BMS-754807.

TABLE 2
Amplification of a region between 13q32-q34 containing of 59 genes
on chromosome 13 showed significant correlation to the IC₅₀
(log 10) values across the CRC cell line panel (p < 0.00005).

| Gene Symbol | Band | r | p-value |
|---|---|---|---|
| IPO5 | 13q32.2 | −0.611141 | 1.34E−05 |
| FARP1 | 13q32.2 | −0.625612 | 7.26E−06 |
| RNF113B | 13q32.2 | −0.594893 | 2.59E−05 |
| STK24 | 13q31.2-q32.3 | −0.640782 | 3.68E−06 |
| SLC15A1 | 13q33-q34 | −0.630951 | 5.74E−06 |
| DOCK9 | 13q32.3 | −0.612174 | 1.29E−05 |
| LOC100129122 | 13q32.3 | −0.583771 | 3.97E−05 |
| UBAC2 | 13q32.3 | −0.607081 | 1.59E−05 |
| UNQ1829 | 13q32.3 | −0.583771 | 3.97E−05 |
| GPR18 | 13q32 | −0.583771 | 3.97E−05 |
| GPR183 | 13q32.3 | −0.583771 | 3.97E−05 |
| TM9SF2 | 13q32.3 | −0.596872 | 2.39E−05 |
| CLYBL | 13q32 | −0.589339 | 3.21E−05 |
| ZIC5 | 13q32.3 | −0.578037 | 4.92E−05 |
| ZIC2 | 13q32 | −0.578037 | 4.92E−05 |
| LOC100131110 | 13q32.3 | −0.57926 | 4.70E−05 |
| A2LD1 | 13q32.3 | −0.581788 | 4.28E−05 |
| TMTC4 | 13q32.3 | −0.581788 | 4.28E−05 |
| IRS2 | 13q34 | −0.598018 | 2.29E−05 |
| LOC728767 | 13q34 | −0.600501 | 2.07E−05 |
| COL4A1 | 13q34 | −0.603545 | 1.83E−05 |
| COL4A2 | 13q34 | −0.62349 | 7.96E−06 |
| LOC100129836 | 13q34 | −0.640839 | 3.67E−06 |
| RAB20 | 13q34 | −0.63104 | 5.72E−06 |
| CARKD | 13q34 | −0.615658 | 1.11E−05 |
| CARS2 | 13q34 | −0.615793 | 1.11E−05 |
| LOC100131435 | 13q34 | −0.611753 | 1.31E−05 |
| ING1 | 13q34 | −0.611753 | 1.31E−05 |
| LOC100129390 | 13q34 | −0.613687 | 1.21E−05 |
| ANKRD10 | 13q34 | −0.613687 | 1.21E−05 |
| ARHGEF7 | 13q34 | −0.636947 | 4.39E−06 |
| C13orf16 | 13q34 | −0.646039 | 2.88E−06 |
| SOX1 | 13q34 | −0.618469 | 9.87E−06 |
| C13orf28 | 13q34 | −0.603571 | 1.83E−05 |
| TUBGCP3 | 13q34 | −0.603522 | 1.83E−05 |
| C13orf35 | 13q34 | −0.625618 | 7.26E−06 |
| ATP11A | 13q34 | −0.659125 | 1.54E−06 |
| MCF2L | 13q34 | −0.642477 | 3.41E−06 |
| F7 | 13q34 | −0.642477 | 3.41E−06 |
| F10 | 13q34 | −0.650253 | 2.36E−06 |
| PROZ | 13q34 | −0.677222 | 6.13E−07 |
| PCID2 | 13q34 | −0.659501 | 1.51E−06 |
| CUL4A | 13q34 | −0.659225 | 1.53E−06 |
| LAMP1 | 13q34 | −0.658935 | 1.55E−06 |
| GRTP1 | 13q34 | −0.647845 | 2.65E−06 |
| ADPRHL1 | 13q34 | −0.65573 | 1.82E−06 |
| DCUN1D2 | 13q34 | −0.65573 | 1.82E−06 |
| TMCO3 | 13q34 | −0.65573 | 1.82E−06 |
| TFDP1 | 13q34 | −0.655776 | 1.81E−06 |
| ATP4B | 13q34 | −0.655776 | 1.81E−06 |
| GRK1 | 13q34 | −0.655776 | 1.81E−06 |
| GAS6 | 13q34 | −0.647695 | 2.67E−06 |
| LOC100128430 | 13q34 | −0.647695 | 2.67E−06 |
| FAM70B | 13q34 | −0.647695 | 2.67E−06 |
| RASA3 | 13q34 | −0.640551 | 3.72E−06 |
| CDC16 | 13q34 | −0.628975 | 6.27E−06 |
| UPF3A | 13q34 | −0.618567 | 9.83E−06 |
| LOC100130463 | 13q34 | −0.628975 | 6.27E−06 |
| ZNF828 | 13q34 | −0.605072 | 1.72E−05 |

### Example 4

### Methods of Assessing Correlation Between IRS2 Expression Level and IGF-1R/IR Inhibitor Sensitivity

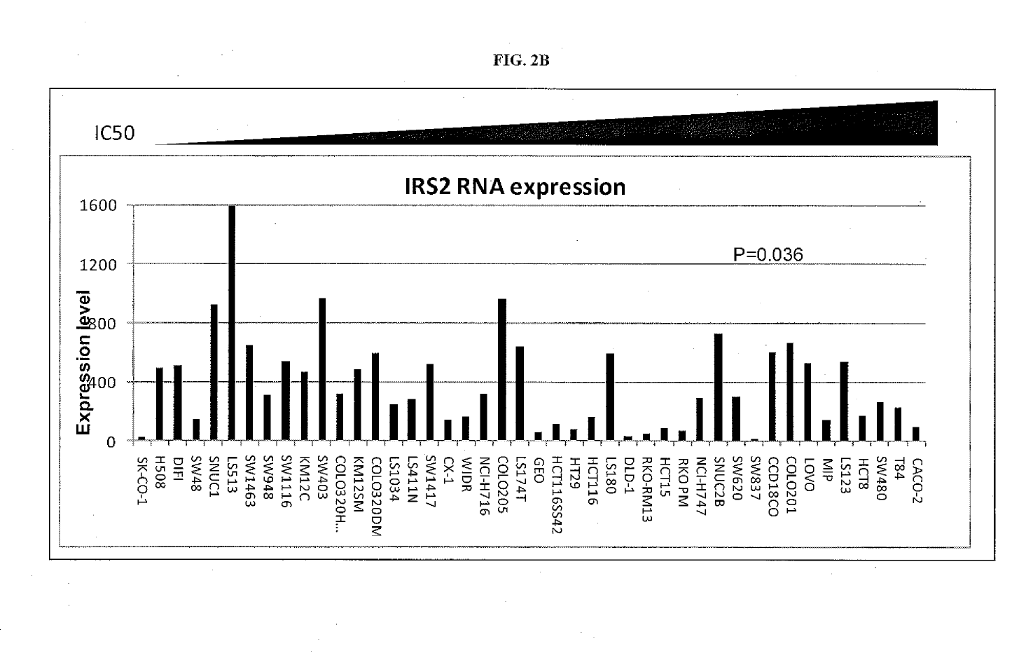

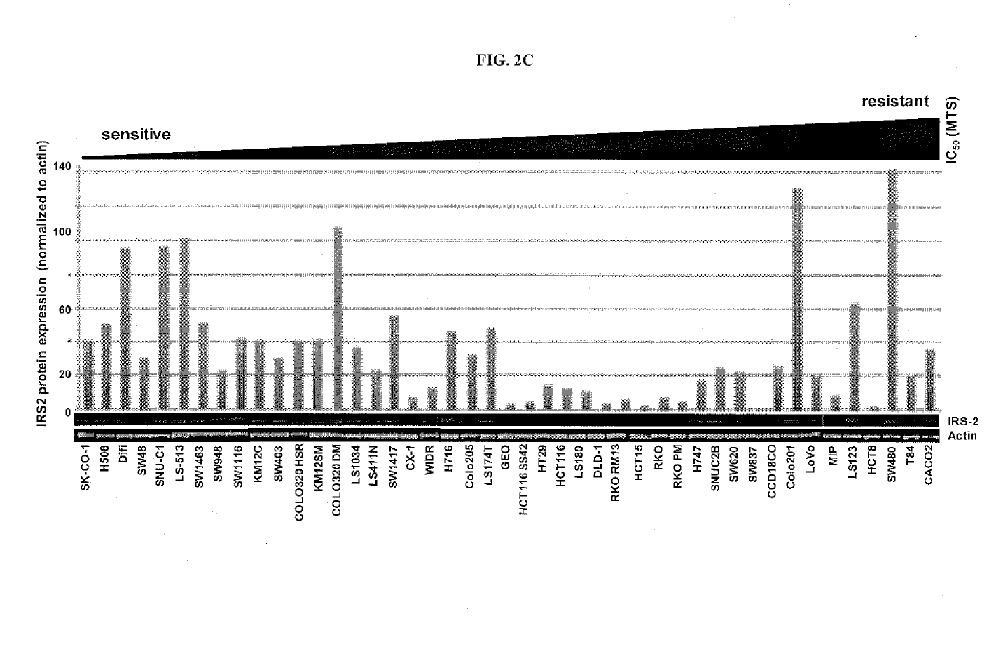

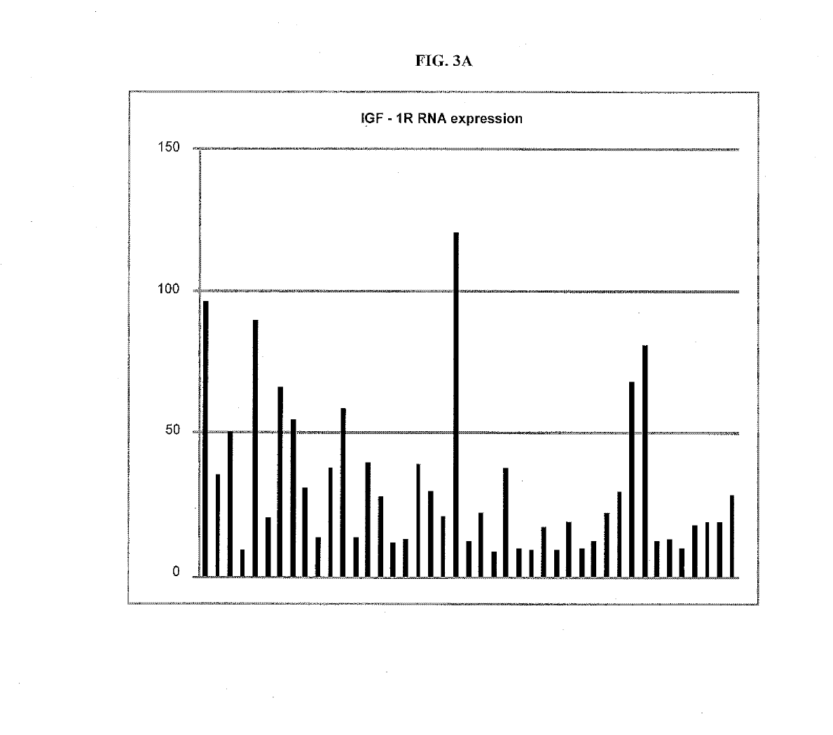

Since IRS2 amplification is correlated with the sensitive to BMS-754807 as shown in FIG. 2A (r=−0.6 and p=0.00002) in CRC cell line panel, the present inventors then looked whether IRS2 DNA amplification is correlated with RNA and protein expression level. Trend towards higher IRS-2 mRNA level (FIGS. 2B and 2C) and protein expression (FIG. 2D) in BMS-754807 sensitive CRC cell lines was observed. Although there is no absolute concordance between IRS2 DNA copy number and RNA expression level, the positive correlation is still significant (r=0.45).

### Example 5

### Methods of Correlating Expression of IGF1R and IGFBP6 with IGF-1R/IR Inhibitor Sensitivity

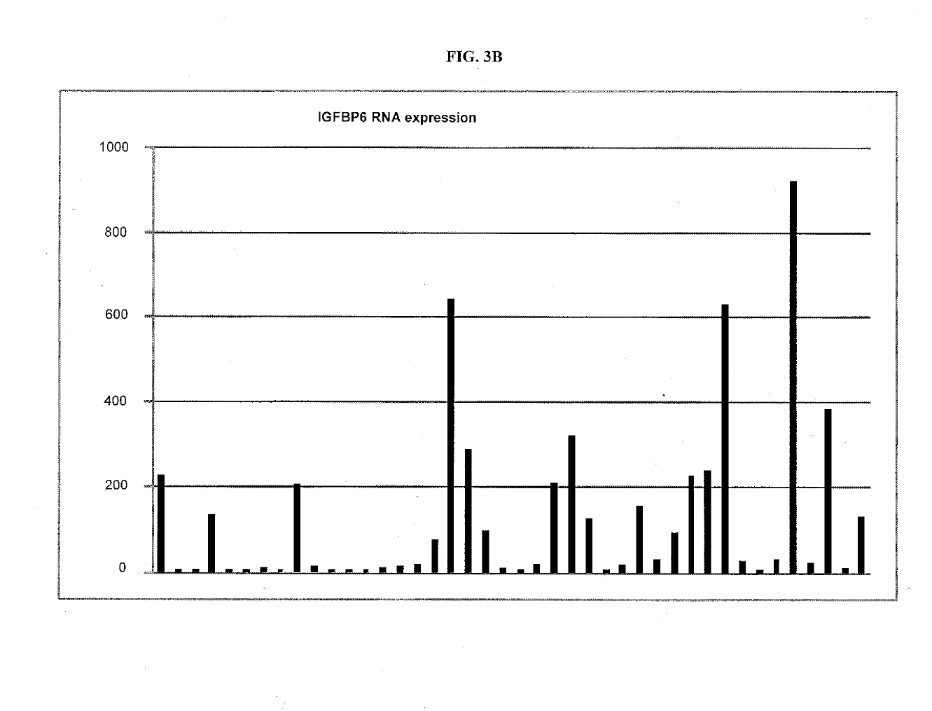

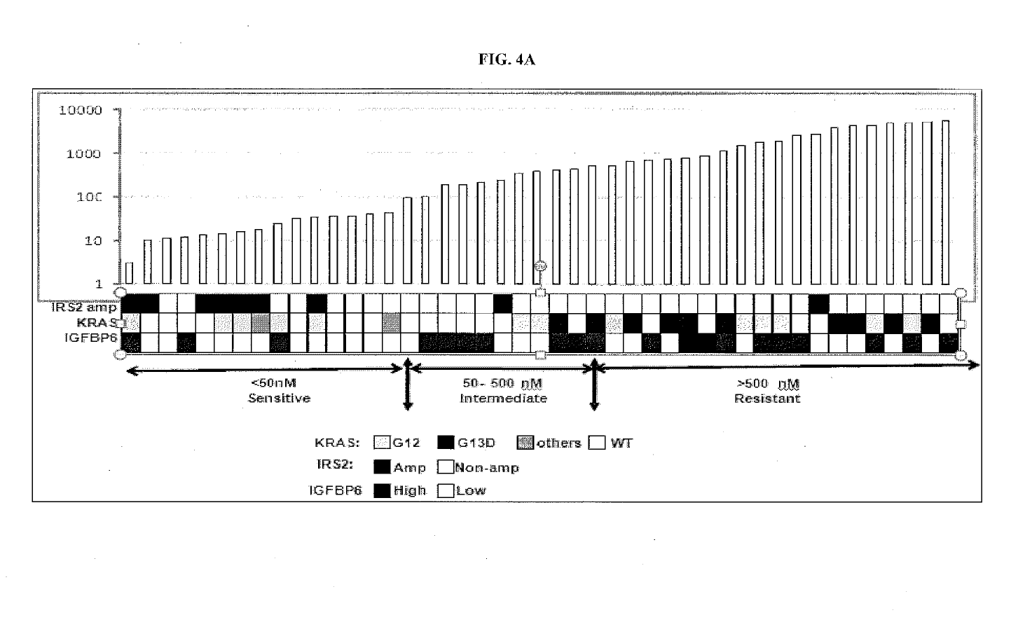

The expression levels of IGF pathway components such as IGF receptors (IGF-1R/IR, IR), ligands (IGF1 and IGF2) and IGF binding proteins (IGFBP1-6) in the CRC panel were evaluated for the association with the sensitivity to BMS-754807. Higher expression level of IGF-1R/IR was seen in the sensitive cell lines (p=0.013 in two sample t-test; FIG. 3A), IGFBP-6 had significant higher expression levels in the resistant cell lines (p=0.0027; FIG. 3B), whereas the expression levels of other components were not correlated with the sensitivity to BMS-754807.

### Example 6

### Methods of Predicting IGF-1R/IR Inhibitor Response Using IRS2 Amplification, KRAS Mutations and IGFBP6 Expression Level

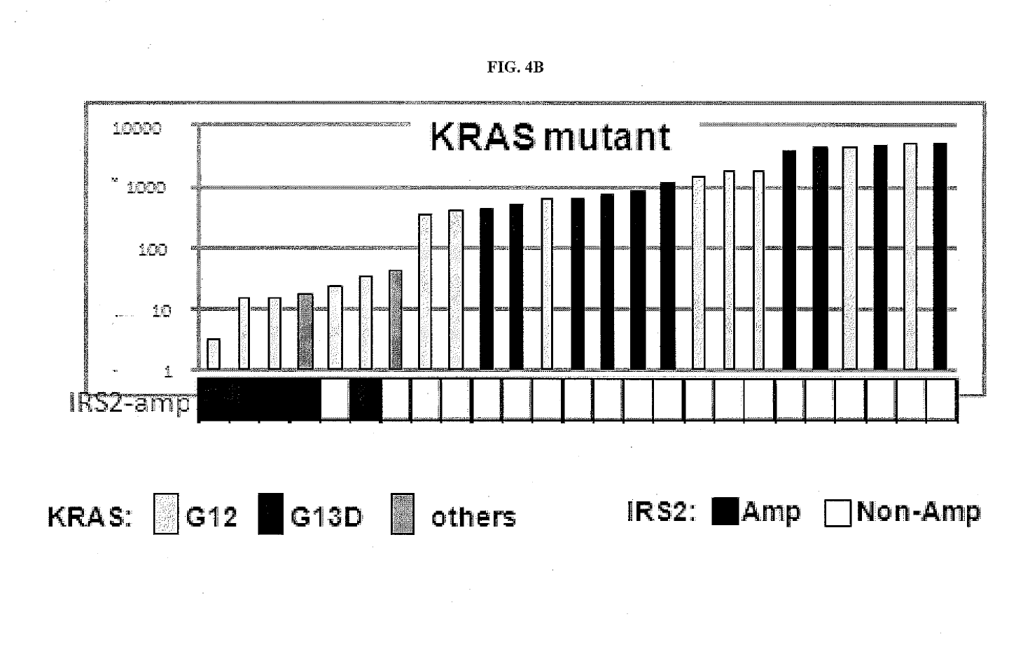

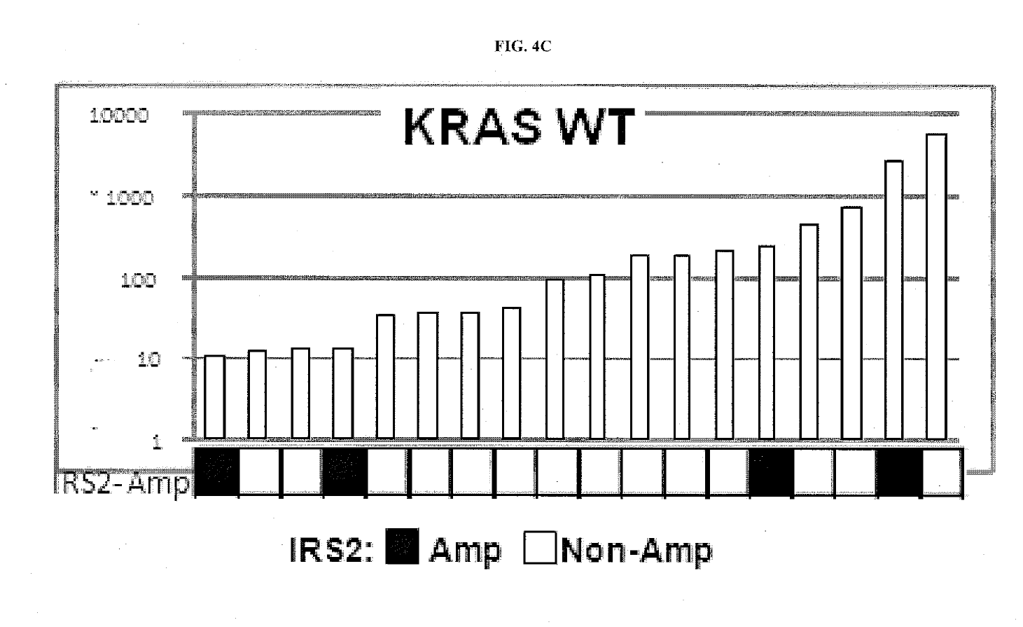

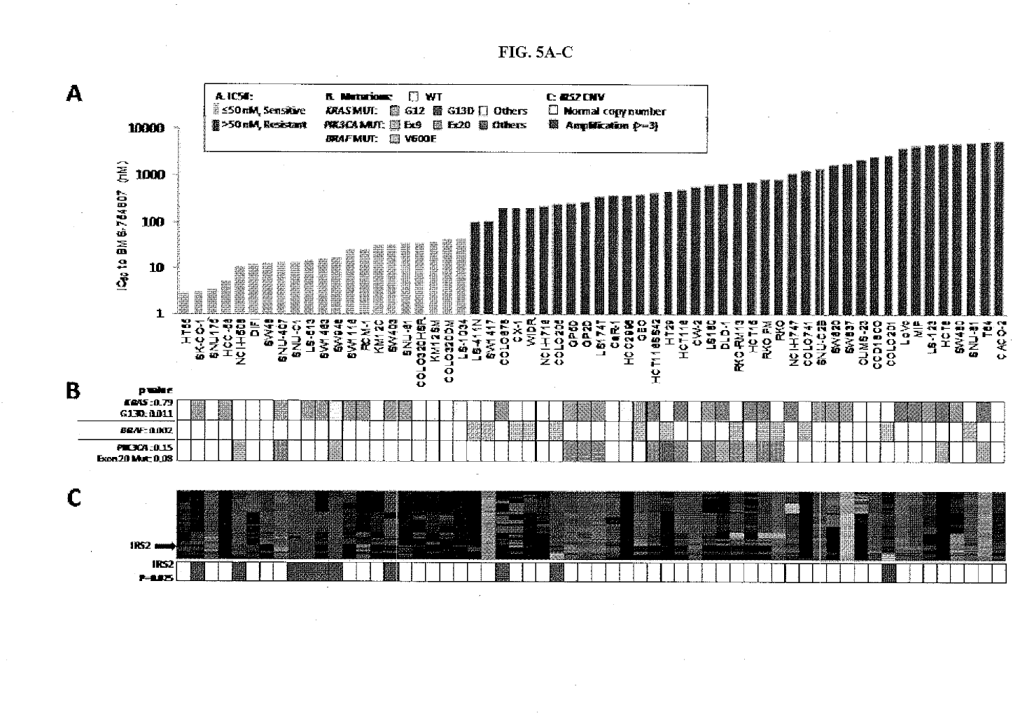

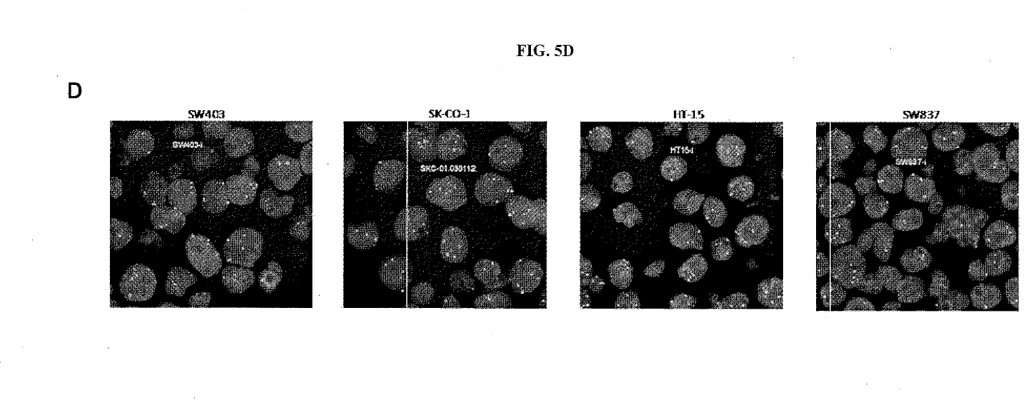

FIG. 4A demonstrated the status of 3 biomarkers in a panel of CRC cell lines: KRAS mutations, IRS2 DNA amplification and IGFBP6 in a panel of CRC cell lines and their relation to the sensitivity to BMS-754807. Cell lines with KRAS mutation, particularly in conden 13 (G13D), are enriched in resistant cell lines. FIG. 4B showed IRS2 DNA amplification and their relation to the sensitivity to BMS-754807 stratified by KRAS status. Cell lines with higher copy of IRS2 are enriched in sensitive cell lines with KRAS mutations, whereas IRS2 amplification is not correlated with BMS-754807 sensitivity in cell lines with KRAS wild type. However, KRAS wild type cell lines with lower expression level of IGFBP6 are enriched in the sensitive group (FIG. 4C). Based on these observations, KRAS mutated cancer patients with IRS2 DNA amplification, or KRAS-wild type samples with the low expression level of IGFBP6 would likely be more responsive to BMS-754807. a combination of KRAS status with either IRS2 copy number or IGFBP6 expression level could be utilized in clinical studies to select patients who are most likely response to BMS-754807.

### Discussion

Because there may be only a subset of patients response to IGF-IR inhibitors, selecting patients most likely to derive clinical benefit could help clinical development of BMS-754807. This study focused on the identification of biomarkers that could be used to guide clinical development of IGF1R/IR inhibitors such as BMS-754807 in CRC. The present inventors conducted pre-clinical pharmacogenomic studies in a panel of colorectal cancer cell lines to identify candidate biomarkers were significantly correlated with the sensitivity/resistance to BMS-754807. Three markers were identified through DNA copy number variation, mutational and gene expression analyses, IRS2 amplification, KRAS mutation and IGFBP6 expression to be correlated to the sensitivity to BMS-754807 in CRC cell lines.

These candidate biomarkers are consistent with biology of the targeted pathway: IRS2 is a cytoplasmic signaling molecule that mediates effects of insulin, IGF-1 by acting as a molecular adaptor between receptor tyrosine kinases and downstream effectors. So the present inventors hypothesize the tumors with IRS2 amplification may indicate the IGF1R/IR pathway activation and tumor's dependence on this pathway for driving proliferation, therefore tumors are more responsive to IGF1R/IR targeting agents. In non-IRS2 amplified tumors with KRAS mutation, the MAKP/ERK pathway is constitutively activated leading to less dependence on IGF1R/IR pathway for proliferation, so less responsive to '807. On the other hand, higher level of IGFBPs may limit bioavailability of 1GF ligands, therefore cause less IGF1R/IR pathway activation. Further testing these biomarkers on whether they are necessary and/or sufficient for the differential sensitivity to agents targeting the IGF signaling pathway is needed to validate the hypotheses.

Further validation of these biomarkers on predictive ability in additional samples is warranted. Afterward, clinical test and validation is needed by a priori screening for IRS2, KARS and IGFBP6 to help stratifying patients likely to benefit from IGF-IR inhibitors in patients with CRC, this should be tested retrospectively in clinical studies and then further validated in perspective studies.

### Example 7

### Method of Assessing the In Vitro Responsiveness of an Expanded Panel of CRC Cell Lines to the IGF-1R/IR Inhibitor BMS-754807

The following experiments relate to and expand upon the experiments described in Example 1.

BMS-754807 is a potent and reversible small molecule TKI with equipotent activity against both IGF-1R and IR. The compound has demonstrated growth inhibition both in vitro and in vivo in multiple tumor types, including CRC (21). Preclinical studies in a panel of ˜200 cell lines from different tumor types revealed that the drug has a dynamic range of activity and a subset of CRC cell lines is very potent to the drug (21). This behavior provides an opportunity for predictive biomarker discovery and suggests that CRC may be a promising indication for IGF-1R/IR TKIs. A more comprehensive genomic approach, including evaluation of gene mutation, DNA copy number, and gene/protein expression, comprehensive review that builds upon the results outlined herein (see Examples 1 thru 6) in order to molecularly characterize a panel of 60 human CRC cell lines. Collectively, these data were then further analyzed in an effort to correlate this expanded panel of CRC cell lines to BMS-754807 response, leading to the identification of candidate predictive biomarkers and hypotheses to be tested during further clinical development of this drug. This expanded investigation confirmed the earlier observations, and also let to some additional correlations that will be outlined further herein.

### Materials and Methods

In vitro Cellular Proliferation Assays. The sources of the 60 human CRC cell lines used in this study are listed in Table 3. Cell proliferation was evaluated by MTS assay after exposure to BMS-754807 for 72 hrs as described previously (22).

Mutational Analysis. KRAS, BRAF, PI3KCA, IGF-1R and IR mutational status of the cell lines was determined from the COSMIC database (23), supplemented with custom sequencing using PCR amplification and sequencing of each exon.

Whole-Genome Copy Number Variation Analysis. The sources of SNP 6.0 array (Affymetrix) data of 60 CRC lines are listed in Table 3. They were either generated from profiling studies according to the Affymetrix protocols or obtained from two public resources: the Cancer Cell Line Encyclopedia (CCLE) project and the Cancer Cell Line Project. Mapping 250K Nsp SNP array (Affymetrix) data for primary CRC tumor samples were obtained from the Gene Expression Omnibus (GEO number GSE16125). The Cel files were processed using the aroma.affymetrix package (24) in the R-project. Segmentation of normalized raw copy number data was performed with the CBS algorithm (2S) implemented in the aroma.affymetrix package. Copy number gain (or loss) of a gene was obtained by using average segmented copy number values within the genomic region of the gene. Copy number profiles were plotted using the Kcsmart package in the Bioconductor project.

Fluorescence In Situ Hybridization (FISH). Cell pellets were prepared from approximately 2×10⁷ cells from each cell line and fixed in 5 mL of 10% neutral buffered formalin at room temperature for 24 hours. Paraffin embedded blocks were made and sections of 3-4 micron thickness were cut. IRS2 copy number of CRC cell lines was tested by FISH assay as developed by Genzyme/LabCorp (Los Angeles, Calif.) using Repeat-Free Poseidon IRS2 (13q34), RB1 (13q14), SE10 triple color probe (catalog # K1-00054, Kreatech, Durham, N.C., USA). Copy number analysis was done in approximately 50 interphase nuclei per sample. Greater than 50% of cells having IRS2 gene>=3 copies were considered as positive for amplification.

qPCR CNV Analysis. The primers and probes for IRS2 and RNaseP were purchased from Applied Biosystems (ABI, Foster City, Calif.; Cat. #4400291 and 4403326). IRS2 gene copy number was detected using the ABI PRISM® 7900HT Sequence Detection System according to manufacture protocols. Copy number was calculated from quadruplet reactions using ABI CopyCaller software, whereby the cycle threshold (CT) of IRS2 was normalized against the CT of an RNaseP reference assay.

qRT-PCR. The TAQMAN® Gene Expression Assay reagents for IRS2 (Cat. #4331182) and β-ACTIN® Cat. #4326315E) genes were purchased from Applied Biosystems-Life Technologies. IR-A expression was also measured using custom primer pairs and probes and used TAQMAN® Gene Expression Assay reagents. The following IR-A sequences were used: IR-A forward: TTTCGTCCCCAGGCCATC (SEQ ID NO:9); IR-A reverse: GCCCGTGAAGTGTCGC (SEQ ID NO:10); and IR-A probe: TTGAGAAGGTGGTGAACA (SEQ ID NO:11). The assays were performed according to the manufacture's protocol.

Affymetrix Gene Array. Expression of IGF1R was measured using the Affymetrix HT_HG-133_Plus_PM array with probes corresponding to NCBI Ref Sequence gi|NM_{—}000875.

Western Blots and MesoScale Discovery (MSD) Multiplex Plate Based Assays. Cell lysates and Western blots were carried out as previously described (21). Antibodies for pIGF-IR/pIR, pAkt, p-p44/42 MAPK and IRS2 for Western blot and MSD were purchased from Cell Signaling Technology and Santa Cruz Biotechnology (see figure legends) except for β-ACTIN® sourced from Millipore. Protein signals from Western blots were visualized using ODYSSEY® Imaging (Li-Cor Biosciences). Measurement of phospho- and total IGF-1R, IR, IRS-1, Akt and MAPK was also determined by commercially available multiplex plate based assays (MSD, Gaithersburg Md.). The assays were performed according to the manufacturer's protocol. Measurement of IRS2 was determined using customized assays utilizing MSD technology.

Small Interfering RNA (siRNA). Cell transfections were carried out using siRNA to human IRS2 (Santa Cruz Biotechnology) with DharmaFECT transfection reagents and Opti-MEM medium (Invitrogen) according to the DharmaFECT General Transfection Protocol. Non-targeting siRNA was transfected into cells as the negative control. After transfection, drug was added to cells and incubated at 37° C. for 72 hours followed by evaluation of cell proliferation as measured by MTS assay.

Statistical Analysis. Categorical data were analyzed by Fisher's exact test. Continuous data were analyzed using Student's t-test. Pearson correlation was used for assessing the correlation between DNA CNV, RNA and protein expression levels, and IC₅₀ values. All differences were considered to be statistically significant for p-values <0.05. Dot plots, bar charts, and box plots were used where appropriate to provide a graphic assessment of the distributions of the data.

### Results

A panel of 60 CRC cell lines were exposed to increasing concentrations of BMS-754807 and assessed for anti-proliferative effects using a MTS assay. The sensitivity is defined by IC₅₀ values, the drug concentration required to achieve 50% growth inhibition. A broad range of sensitivity to BMS-754807 was observed, ranging from 0.003-5.5 μmol/L (see Table 3). Twenty-one cell lines with IC₅₀≦50 nmol/L were defined as sensitive and all other lines with IC₅₀>50 nmol/L were defined as resistant (FIG. 5A). Although the demarcation for sensitivity is arbitrary, PK data from phase I solid tumor clinical trials showed that 50 nmol/L of the drug was below the average plasma concentration of BMS-754807 at steady state achieved in patients at minimum efficacious dose, which was determined by a preclinical CRC xenograft model (26, 27), suggesting the concentration used for sensitivity demarcation is clinically relevant and achievable. These expanded results are consistent with the preliminary results observed in Example 1 herein.

### Tables 3A-B

Compilation of the in vitro sensitivity profiles of BMS-754807, mutational status of key cancer driver genes, IRS2 DNA copy number and sources for SNP data, protein and RNA expression data for IRS2, RNA expression data for IR-A, IGF-1R and IGFBP6 in a panel of 60 CRC cell lines used in this study. Table 3A provides RNA expression analysis results, while Table 3B provides gene mutation results.

TABLE 3A

| | | | | | RNA Expression Level | | | |
|---|---|---|---|---|---|---|---|---|
| | | IC₅₀, | Sensitivity | IRS2 Protein | IRS2 by | IR-A by | IGF-1R by | IGFBP6 by |
| Cell Line | Source of Cell Line | nM | Classes^{a} | level | RT-PCR | RT-PCR | Affymetrix | Affymetrix |
| HT55 | HPA | 3 | S | 16157.8 | 57.6 | 6.0 | 32.7 | 12.5 |
| SK-CO-1 | ATCC | 3 | S | 4558 | 12.6 | 3.1 | 43.5 | 61.3 |
| SNU175 | KCLB | 3 | S | 14105.5 | 75.1 | 9.2 | 18.0 | 9.6 |
| HCC-56 | JCRB/HSRRB | 5 | S | 16332.4 | 39.5 | 3.4 | 43.2 | 9.3 |
| NCI-H508 | ATCC | 11 | S | 8839 | 156.3 | 8.6 | 26.6 | 9.0 |
| DIFI | Investigator | 12 | S | 11862 | 24.8 | 1.5 | 32.0 | 9.9 |
| SW48 | ATCC | 13 | S | 3985 | 16.4 | 7.0 | 10.6 | 50.4 |
| SNU-407 | KCLB | 13 | S | 4730.7 | 26.2 | 4.1 | 16.4 | 27.4 |
| SNU-C1 | ATCC | 13 | S | 11849.5 | 35.6 | 1.1 | 42.0 | 11.4 |
| LS-513 | ATCC | 15 | S | 19845.5 | 67.4 | 2.3 | 19.3 | 9.6 |
| SW1463 | ATCC | 16 | S | 18257 | 161.1 | 3.2 | 36.5 | 12.5 |
| SW948 | ATCC | 18 | S | 11070.5 | 21.2 | 2.2 | 33.5 | 10.3 |
| SW1116 | ATCC | 24 | S | 11166.5 | 29.5 | 1.3 | 24.6 | 59.4 |
| RCM-1 | JCRB/HSRRB | 24 | S | 5927.7 | 4.3 | 0.4 | 39.1 | 64.0 |
| KM12C | Investigator | 33 | S | 8463 | 62.3 | 12.9 | 14.5 | 17.4 |
| SW403 | ATCC | 34 | S | 10464.5 | 18.7 | 2.1 | 27.5 | 8.6 |
| SNU-61 | KCLB | 35 | S | 8246.3 | 46.1 | 9.2 | 45.9 | 19.2 |
| COLO-320-HSR | ATCC | 35 | S | 3854.5 | 78.9 | 6.8 | 34.5 | 9.5 |
| KM12SM | Investigator | 37 | S | 8362.5 | 16.0 | 1.3 | 14.6 | 9.6 |
| COLO320DM | ATCC | 41 | S | 8839.5 | 80.8 | 4.8 | 28.3 | 12.7 |
| LS-1034 | ATCC | 44 | S | 8746 | 17.6 | 5.6 | 23.3 | 17.2 |
| LS-411N | ATCC | 99 | R | 6800.5 | 13.9 | 3.0 | 13.0 | 19.2 |
| SW1417 | ATCC | 106 | R | 14651.5 | 51.7 | 7.0 | 13.8 | 39.4 |
| COLO-678 | DSMZ | 192 | R | 3378.2 | 1.8 | 0.5 | 33.2 | 64.5 |
| CX-1 | Investigator | 194 | R | 12396 | 15.3 | 2.7 | 28.1 | 87.0 |
| WIDR | ATCC | 194 | R | 4472 | 13.2 | 2.6 | 23.9 | 67.1 |
| NCI-H716 | ATCC | 217 | R | 11645.5 | 6.3 | 0.9 | 19.6 | 44.4 |
| COLO-205 | ATCC | 242 | R | 20129.5 | 27.8 | 1.5 | 47.8 | 12.1 |
| GP5D | HPA | 252 | R | 5436.2 | 17.1 | 5.0 | 14.2 | 12.3 |
| GP2D | HPA | 261 | R | 7890.3 | 7.1 | 1.8 | 14.8 | 11.1 |
| LS174T | ATCC | 358 | R | 10544 | 45.5 | 11.4 | 13.3 | 9.3 |
| CaR-1 | JCRB/HSRRB | 375 | R | 11904.3 | 28.9 | 0.1 | 57.1 | 62.9 |
| HCC2998 | NCI DTP, DCTD | 380 | R | 5667.9 | 6.3 | 0.6 | 31.2 | 54.5 |
| | Tumor repository | | | | | | | |
| GEO | Investigator | 407 | R | 2551 | 10.4 | 0.8 | 20.2 | 20.3 |
| HCT116SS42 | Investigator | 427 | R | 1371 | 5.1 | 8.3 | 10.0 | 59.5 |
| HT-29 | ATCC | 451 | R | 6727.5 | 6.0 | 2.5 | 27.6 | 69.5 |
| HCT-116 | ATCC | 503 | R | 3974.5 | 20.6 | 9.8 | 11.1 | 49.0 |
| CW-2 | RIKEN | 586 | R | 8382.1 | 25.3 | 2.2 | 16.0 | 53.4 |
| LS-180 | ATCC | 629 | R | 11976.5 | 27.0 | 3.4 | 10.6 | 10.7 |
| DLD-1 | ATCC | 666 | R | 1672.5 | 8.4 | 0.9 | 17.2 | 18.8 |
| RKO-RM13 | Investigator | 727 | R | 2564.5 | 2.4 | 1.8 | 10.4 | 53.4 |
| HCT-15 | ATCC | 757 | R | 1143.5 | 9.3 | 7.5 | 18.4 | 25.4 |
| RKO PM | Investigator | 857 | R | 2609 | 2.8 | 2.0 | 10.9 | 43.8 |
| RKO | ATCC | 857 | R | 1848 | 6.6 | 2.6 | 20.9 | 81.0 |
| NCI-H747 | ATCC | 1155 | R | 5387 | 23.0 | 1.5 | 20.2 | 70.7 |
| COLO741 | HPA | 1350 | R | 15555 | 11.9 | 0.7 | 63.6 | 21.1 |
| SNU-C2B | ATCC | 1480 | R | 15700 | 70.9 | 7.5 | 13.2 | 20.6 |
| SW620 | ATCC | 1832 | R | 18035.5 | 25.2 | 2.3 | 20.3 | 61.6 |
| SW837 | ATCC | 1886 | R | 1247 | 5.3 | 8.0 | 24.0 | 62.7 |
| OUMS-23 | JCRB/HSRRB | 2217 | R | 1214.6 | 1.0 | 0.6 | 36.4 | 32.0 |
| CCD18CO | ATCC | 2555 | R | 5193.5 | 13.3 | 0.1 | 37.1 | 86.6 |
| COLO-201 | ATCC | 2760 | R | 20089.5 | 23.9 | 2.1 | 40.1 | 23.7 |
| LoVo | ATCC | 3871 | R | 8577.5 | 64.9 | 3.2 | 13.2 | 9.9 |
| MIP | Investigator | 4359 | R | 1976 | 7.8 | 1.0 | 13.8 | 25.3 |
| LS-123 | ATCC | 4473 | R | 12777.5 | 12.0 | 0.7 | 11.0 | 97.0 |
| HCT8 | ATCC | 4855 | R | 6103 | 3.7 | 1.4 | 17.6 | 22.0 |
| SW480 | ATCC | 4888 | R | 20089 | 28.9 | 3.1 | 18.1 | 73.9 |
| SNU-81 | KCLB | 5000 | R | 1998.5 | 4.8 | 3.3 | 27.2 | 52.7 |
| T84 | ATCC | 5200 | R | 333 | 7.4 | 1.9 | 18.3 | 14.9 |
| CACO-2 | ATCC | 5496 | R | 3524.5 | 3.3 | 0.8 | 23.5 | 49.7 |

TABLE 3B

| | | | | | | | | IRS2 | | |
|---|---|---|---|---|---|---|---|---|---|---|
| | IC₅₀, | Sensitivity | Mutation Status | | | | | Copy | Source of | |
| Cell Line | nM | Classes^{a} | KRAS | BRAF | PIK3CA ^{b, c} | IGF-1R^{d} | IR^{d} | Number^{e} | SNP Data | FISH^{f} |
| HT55 | 3 | S | WT | WT | WT | | | 2.2 | Sanger | EQUIVOCAL |
| SK-CO-1 | 3 | S | G12V | WT | WT | WT | WT | 3 | Internal | POSITIVE |
| SNU175 | 3 | S | WT | WT | WT | | | 1.9 | CCEL | |
| HCC-56 | 5 | S | G12V | WT | WT | WT | WT | 2.4 | CCEL | NEGATIVE |
| NCI-H508 | 11 | S | WT | WT | E545K ^{b} | WT | WT | 3.3 | Sanger | |
| DIFI | 12 | S | WT | WT | WT | | | 2.9 | Internal | |
| SW48 | 13 | S | WT | WT | WT | A828T | WT | 2.2 | Internal | |
| SNU-407 | 13 | S | G12D | WT | H1047K^{c} | | | 1.9 | CCEL | |
| SNU-C1 | 13 | S | WT | WT | WT | WT | WT | 3.5 | Internal | |
| LS-513 | 15 | S | G12D | WT | WT | WT | WT | 3.9 | Internal | POSITIVE |
| SW1463 | 16 | S | G12C | WT | WT | WT | WT | 3.1 | Sanger | POSITIVE |
| SW948 | 18 | S | Q61L | WT | E542K ^{b} | WT | WT | 3.4 | Internal | POSITIVE |
| SW1116 | 24 | S | G12D | WT | WT | WT | WT | 2.3 | Internal | EQUIVOCAL |
| RCM-1 | 24 | S | G12V | WT | WT | WT | WT | 1.7 | Sanger | NEGATIVE |
| KM12C | 33 | S | WT | WT | WT | WT | R1270C | 2.3 | Internal | |
| SW403 | 34 | S | G12V | WT | WT | | | 3.5 | Internal | POSITIVE |
| SNU-61 | 35 | S | G12D | WT | WT | | | 2.3 | CCEL | |
| COLO-320-HSR | 35 | S | WT | WT | WT | WT | WT | 2.7 | Internal | |
| KM12SM | 37 | S | WT | WT | WT | | | 2.3 | Internal | |
| COLO320DM | 41 | S | WT | WT | WT | | | 2.7 | Internal | |
| LS-1034 | 44 | S | A146T | WT | WT | WT | WT | 2.7 | Internal | EQUIVOCAL |
| LS-411N | 99 | R | WT | V600E | WT | R461H | WT | 2.3 | Sanger | |
| SW1417 | 106 | R | WT | V600E | WT | S72N | WT | 1.6 | Internal | |
| COLO-678 | 192 | R | G12D | WT | WT | WT | WT | 3 | Sanger | NEGATIVE |
| CX-1 | 194 | R | WT | V600E | WT | | | 2.3 | Internal | NEGATIVE |
| WIDR | 194 | R | WT | V600E | WT | | | 2.5 | Internal | EQUIVOCAL |
| NCI-H716 | 217 | R | WT | WT | WT | WT | WT | 2.3 | Sanger | |
| COLO-205 | 242 | R | WT | V600E | WT | WT | WT | 3.7 | Internal | |
| GP5D | 252 | R | G12D | WT | H1047L | Y201H; | WT | 1.9 | Sanger | NEGATIVE |
| | | | | | | D435G | | | | |
| GP2D | 261 | R | G12D | WT | H1047L | | | 2 | Sanger | |
| LS174T | 358 | R | G12D | WT | H1047R | | | 2 | Internal | NEGATIVE |
| CaR-1 | 375 | R | WT | WT | WT | | | 2.2 | Sanger | POSITIVE |
| HCC2998 | 380 | R | A146T | WT | WT | WT | WT | 2.4 | Sanger | EQUIVOCAL |
| GEO | 407 | R | G12A | V600E | WT | | | 1.9 | Internal | NEGATIVE |
| HCT116SS42 | 427 | R | G13D | WT | H1047R | | | 2.1 | Internal | NEGATIVE |
| HT-29 | 451 | R | WT | V600E | P449T | WT | WT | 2.6 | Internal | |
| HCT-116 | 503 | R | G13D | WT | H1047R | WT | WT | 2.1 | Internal | NEGATIVE |
| CW-2 | 586 | R | WT | WT | WT | WT | Stop | 1.9 | Sanger | |
| LS-180 | 629 | R | G12D | WT | H1047R | WT | WT | 2.2 | Internal | NEGATIVE |
| DLD-1 | 666 | R | G13D | WT | E545K ^{b} | | | 2.3 | Internal | NEGATIVE |
| RKO-RM13 | 727 | R | WT | V600E | H1047R | | | 2.1 | Internal | |
| HCT-15 | 757 | R | G13D | WT | D549N ^{b}, | N792D; | K337N; | 2.3 | Internal | NEGATIVE |
| | | | | | E545K | R1246C | F258S | | | |
| RKO PM | 857 | R | G13D | V600E | H1047R | | | 2.2 | Internal | NEGATIVE |
| RKO | 857 | R | WT | V600E | H1047R | WT | WT | 2 | Sanger | |
| NCI-H747 | 1155 | R | G13D | WT | WT | WT | WT | 2.1 | Sanger | NEGATIVE |
| COLO741 | 1350 | R | WT | V600E | WT | WT | WT | 2.4 | Sanger | EQUIVOCAL |
| SNU-C2B | 1480 | R | G12D | WT | WT | WT | R1128H | 2 | Internal | NEGATIVE |
| SW620 | 1832 | R | G12V | WT | WT | WT | WT | 2.7 | Internal | EQUIVOCAL |
| SW837 | 1886 | R | G12C | WT | WT | WT | WT | 1.4 | Internal | NEGATIVE |
| OUMS-23 | 2217 | R | WT | WT | WT | | | 2.6 | CCEL | |
| CCD18CO | 2555 | R | WT | WT | WT | | | 1.8 | Internal | |
| COLO-201 | 2760 | R | WT | V600E | WT | | | 3.2 | Internal | |
| LoVo | 3871 | R | G13D | WT | WT | WT | WT | 1.9 | Internal | NEGATIVE |
| MIP | 4359 | R | G13D | WT | WT | | | 2 | Internal | EQUIVOCAL |
| LS-123 | 4473 | R | G12S | WT | WT | WT | WT | 2.1 | Internal | EQUIVOCAL |
| HCT8 | 4855 | R | G13D | WT | E545K ^{b} | | | 2.4 | Internal | NEGATIVE |
| SW480 | 4888 | R | G12V | WT | WT | | | 1.7 | Internal | NEGATIVE |
| SNU-81 | 5000 | R | WT | V600E | WT | | | 1.9 | CCEL | |
| T84 | 5200 | R | G13D | WT | E542K ^{b} | WT | V774M | 1.6 | Internal | NEGATIVE |
| CACO-2 | 5496 | R | WT | WT | WT | | | 2.1 | Internal | |

^{a}“S” refers Sensitive; “R” refers Resistant
^{b} PIK3CA activating mutations in exon 9
^{c} PIK3CA activating mutations in exon 20
^{d}Blank refers “Not Tested”
^{e}IRS2 copy number >= 3 defined as amplification
^{f}Blank refers “Not Tested”. The FISH results are defined: “Positive” as IRS2 => 3 copies in =>50% cells; “Equivocal” in 25%-49% cells; “Negative” in ≦24% cells

### Example 8

### Expanded Methods for Analyzing Association Between Cancer Driven Mutations and Pathway Related Gene Alterations with Sensitivity to BMS-754807

The following experiments relate to and expand upon the experiments described in Example 2. Materials and methods for these experiments are as described in Example 7 herein.

In vitro proliferation results have indicated approximately 30% of the CRC lines tested were sensitive to BMS-754807, providing an opportunity for predictive biomarker discovery. First, the present inventors characterized the genetic status of selected key cancer driver mutations for CRC, and correlated the mutational status of KRAS, BRAF and PIK3CA with the sensitivity of BMS-754807 (FIG. 5B). The Fisher exact test (Table 4) demonstrated that the association between BMS-754807 sensitivity and BRAF mutational status was significant (p=0.002), and that all cell lines harboring BRAF^{V600E} mutations were resistant. The association was not statistically significant for mutational status of KRAS (p=0.79) or PIK3CA (p=0.15). However, when mutations at different amino acid positions were assessed, the present inventors found that all 10 cell lines with KRAS^{G13D} mutations were resistant to the drug (p=0.011), while mutations on codon 12 did not correlate with drug sensitivity (p=0.27). In addition, 9 of 10 cell lines with PIK3CA activating mutations within exon 20 were resistant and only 1 was sensitive (p=0.08). Furthermore, 10 of the 16 cell lines wild type (WT) for both KRAS/BRAF were sensitive to BMS-754807 (p=0.013).

Mutational status for other cancer drivers such as p53, PTEN and APC were not found to be correlated with sensitivity to BMS-754807 (data not shown). Sequencing the drug target genes IGF-1R and IR in a subset of cell lines did not uncover any mutational hot spots, and the detected mutations in these two genes did not reveal significant association with the drug sensitivity (Table 3), which is consistent with a previous report that IGF-1R mutations in CRC had no association with the sensitivity to IGF-1R antibody, figitumumab (28). These expanded results are consistent with the preliminary results observed in Example 2 herein.

TABLE 4
The association between mutational status of KRAS, BRAF
and PIK3CA and BMS-754807 sensitivity as evaluated by
Fisher exact test in a panel of 60 CRC cell lines.

| | | | p value, |
|---|---|---|---|
| | Sensitive | Resistant | Fisher-exact test |
| KRAS mutation | 11 | 22 | 0.79 |
| WT | 10 | 17 | |
| KRAS G13V | 0 | 10 | 0.011 |
| WT & other mutation | 21 | 29 | |
| KRAS G12 | 9 | 11 | 0.27 |
| WT & other mutation | 12 | 28 | |
| BRAF V600E | 0 | 13 | 0.002 |
| WT | 21 | 26 | |
| KRAS or BRAF mutation | 11 | 33 | 0.013 |
| WT/WT | 10 | 6 | |
| PI3KCA activating mutation | 3 | 14 | 0.13 |
| WT | 18 | 25 | |
| PI3KCA mutation Exon 9 | 2 | 4 | 1 |
| WT | 19 | 35 | |
| PI3KCA mutation Exon 20 | 1 | 9 | 0.08 |
| WT | 20 | 30 | |

### Example 9

### Expanded Methods for Associating Copy Number Alteration with Sensitivity to BMS-754807 Through Whole-Genome Copy Number Variation (CNV) Analysis

The following experiments relate to and expand upon the experiments described in Example 3. Materials and methods for these experiments are as described in Example 7 herein.

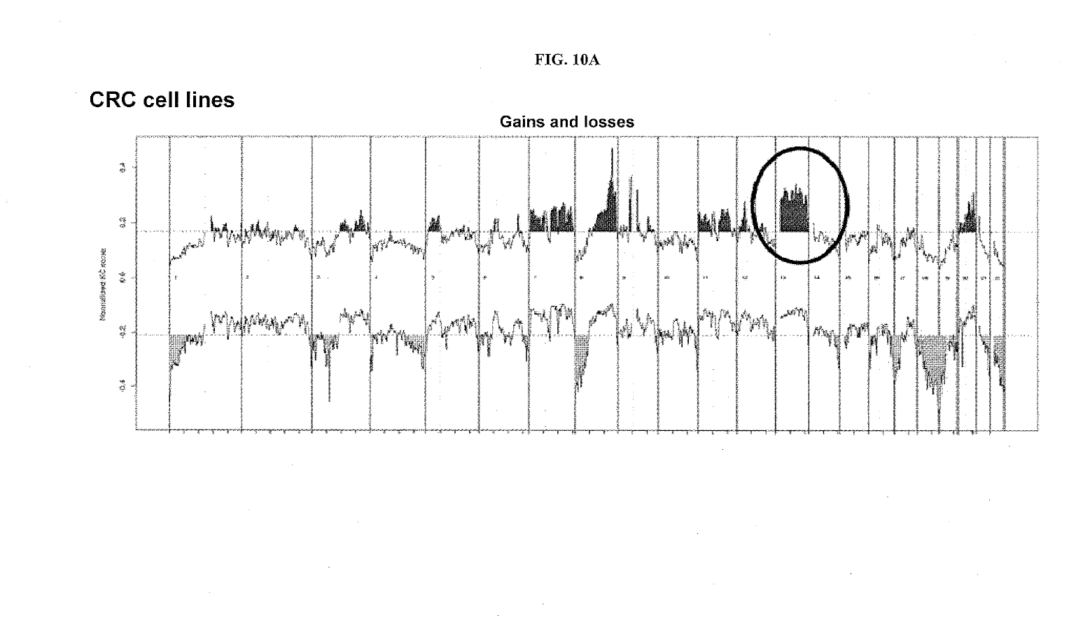

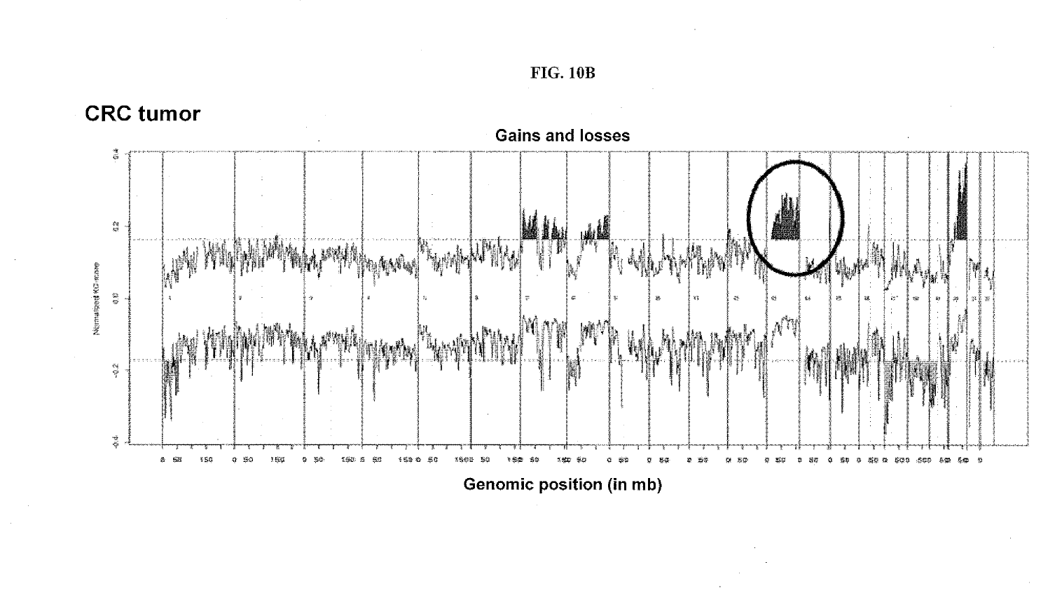

Genome-wide analysis of copy number gain and loss using Affymetrix SNP6.0 microarray data was performed to determine whether there was any CNV associated with in vitro sensitivity to BMS-754807. Two statistical analyses were performed including Pearson correlation with IC₅₀ values, and a student t-test to compare sensitive to resistant cell lines. This led to the identification of genomic segments of 197 genes that showed variation in DNA copy number significantly associated with the drug sensitivity (p<0.005 in both statistical tests), and interestingly they are all located on chromosome 13 (see Tables 5A-B). Cell lines with chromosome 13 gene amplifications were enriched in the sensitive group (FIG. 5C). To test whether the chromosome 13 copy number gain observation was an artifact of cell-line models, the present inventors evaluated and compared the DNA CNV profiles in CRC cell lines to those in a published dataset of CRC primary tumor samples (29), and found that the profiles in both were very similar (FIG. 10), confirming that copy number gain on segments of chromosome 13 are a frequent event in CRC tumors.

Among those genes amplified, IRS2 encodes for the downstream substrate of both IGF-1R and IR signaling pathways, and as indicated in FIG. 1C, 7 of the 10 cell lines with IRS2 amplification (>=3 copy number) were sensitive, while 3 lines were resistant to BMS-754807 (p=0.025). To confirm the IRS2 amplification results from SNP analysis, fluorescent in situ hybridization (FISH) assay was performed on 35 lines; the concordance of IRS2 amplification status was 94% (see Table 3) and the FISH results on representative cell lines were shown on FIG. 5D. These expanded results are consistent with the preliminary results observed in Example 3 herein.

### Tables 5A-B

Genes are listed with DNA copy number variation (CNV) that have been associated with in vitro sensitivity to BMS-754807 in all 60 CRC cell lines, in KRAS wild type or in KRAS mutated CRC cell lines. Table 5A provides the Pearson correlation results, while Table 5B provides the T-test results.

TABLE 5A

| | | | | Pearson correlation | | | | | |
|---|---|---|---|---|---|---|---|---|---|
| | | | | in all lines | | in KRAS-MUT | | in KRAS-WT | |
| | | | | (n = 60) | | only (n = 33) | | only (n = 27) | |
| Locus | Symbol | Description | Band | r | p-value | r | p-value | r | p-value |
| 143 | PARP4 | poly (ADP-ribose) polymerase | 13q11 | −0.385 | 0.0024 | −0.488 | 0.0039 | −0.251 | 0.207 |
| | | family, member 4 | | | | | | | |
| 241 | ALOX5AP | arachidonate 5-lipoxygenase- | 13q12 | −0.373 | 0.0033 | −0.467 | 0.0062 | −0.252 | 0.205 |
| | | activating protein | | | | | | | |
| 479 | ATP12A | ATPase, H+/K+ transporting, | 13q12.12\|13q12.1- | −0.377 | 0.0030 | −0.469 | 0.0059 | −0.251 | 0.207 |
| | | nongastric, alpha polypeptide | q12.3 | | | | | | |
| 496 | ATP4B | ATPase, H+/K+ exchanging, beta | 13q34 | −0.460 | 0.0002 | −0.519 | 0.0020 | −0.409 | 0.034 |
| | | polypeptide | | | | | | | |
| 540 | ATP7B | ATPase, Cu++ transporting, beta | 13q14.3 | −0.370 | 0.0036 | −0.526 | 0.0017 | −0.114 | 0.570 |
| | | polypeptide | | | | | | | |
| 675 | BRCA2 | breast cancer 2, early onset | 13q12.3 | −0.389 | 0.0021 | −0.485 | 0.0042 | −0.271 | 0.172 |
| 1024 | CDK8 | cyclin-dependent kinase 8 | 13q12 | −0.379 | 0.0028 | −0.488 | 0.0039 | −0.232 | 0.245 |
| 1102 | RCBTB2 | regulator of chromosome | 13q14.3 | −0.370 | 0.0036 | −0.548 | 0.0010 | −0.111 | 0.580 |
| | | condensation (RCC1) and BTB | | | | | | | |
| | | (POZ) domain containing protein | | | | | | | |
| | | 2 | | | | | | | |
| 1282 | COL4A1 | collagen, type IV, alpha 1 | 13q34 | −0.388 | 0.0022 | −0.534 | 0.0014 | −0.150 | 0.455 |
| 1284 | COL4A2 | collagen, type IV, alpha 2 | 13q34 | −0.387 | 0.0023 | −0.527 | 0.0016 | −0.171 | 0.394 |
| 1361 | CPB2 | carboxypeptidase B2 (plasma) | 13q14.11 | −0.381 | 0.0026 | −0.582 | 0.0004 | −0.123 | 0.540 |
| 1638 | DCT | dopachrome tautomerase | 13q32 | −0.369 | 0.0037 | −0.574 | 0.0005 | −0.075 | 0.709 |
| | | (dopachrome delta-isomerase, | | | | | | | |
| | | tyrosine-related protein 2) | | | | | | | |
| 1880 | GPR183 | G protein-coupled receptor 183 | 13q32.3 | −0.405 | 0.0013 | −0.577 | 0.0004 | −0.183 | 0.360 |
| 1948 | EFNB2 | ephrin-B2 | 13q33 | −0.390 | 0.0021 | −0.611 | 0.0002 | −0.063 | 0.753 |
| 2073 | ERCC5 | excision repair cross- | 13q33 | −0.377 | 0.0030 | −0.610 | 0.0002 | −0.044 | 0.827 |
| | | complementing rodent repair | | | | | | | |
| | | deficiency, complementation | | | | | | | |
| | | group 5 | | | | | | | |
| 2098 | ESD | esterase D/formylglutathione | 13q14.1-q14.2 | −0.371 | 0.0035 | −0.561 | 0.0007 | −0.123 | 0.540 |
| | | hydrolase | | | | | | | |
| 2155 | F7 | coagulation factor VII (serum | 13q34 | −0.416 | 0.0010 | −0.519 | 0.0020 | −0.263 | 0.185 |
| | | prothrombin conversion | | | | | | | |
| | | accelerator) | | | | | | | |
| 2159 | F10 | coagulation factor X | 13q34 | −0.416 | 0.0010 | −0.519 | 0.0020 | −0.263 | 0.185 |
| 2254 | FGF9 | fibroblast growth factor 9 (glia- | 13q11-q12 | −0.374 | 0.0032 | −0.480 | 0.0047 | −0.216 | 0.278 |
| | | activating factor) | | | | | | | |
| 2308 | FOXO1 | forkhead box O1 | 13q14.1 | −0.367 | 0.0040 | −0.495 | 0.0034 | −0.168 | 0.401 |
| 2621 | GAS6 | growth arrest-specific 6 | 13q34 | −0.459 | 0.0002 | −0.519 | 0.0020 | −0.412 | 0.033 |
| 2700 | GJA3 | gap junction protein, alpha 3, | 13q11-q12 | −0.375 | 0.0032 | −0.488 | 0.0040 | −0.222 | 0.265 |
| | | 46 kDa | | | | | | | |
| 2706 | GJB2 | gap junction protein, beta 2, | 13q11-q12 | −0.378 | 0.0029 | −0.491 | 0.0037 | −0.222 | 0.265 |
| | | 26 kDa | | | | | | | |
| 2835 | GPR12 | G protein-coupled receptor 12 | 13q12 | −0.378 | 0.0029 | −0.513 | 0.0023 | −0.203 | 0.310 |
| 2841 | GPR18 | G protein-coupled receptor 18 | 13q32 | −0.405 | 0.0013 | −0.577 | 0.0004 | −0.183 | 0.360 |
| 2963 | GTF2F2 | general transcription factor IIF, | 13q14 | −0.390 | 0.0021 | −0.583 | 0.0004 | −0.122 | 0.545 |
| | | polypeptide 2, 30 kDa | | | | | | | |
| 3146 | HMGB1 | high-mobility group box 1 | 13q12 | −0.375 | 0.0032 | −0.455 | 0.0078 | −0.260 | 0.190 |
| 3356 | HTR2A | 5-hydroxytryptamine (serotonin) | 13q14-q21 | −0.371 | 0.0035 | −0.561 | 0.0007 | −0.123 | 0.540 |
| | | receptor 2A | | | | | | | |
| 3621 | ING1 | inhibitor of growth family, | 13q34 | −0.397 | 0.0017 | −0.535 | 0.0013 | −0.183 | 0.361 |
| | | member 1 | | | | | | | |
| 3843 | IPO5 | importin 5 | 13q32.2 | −0.435 | 0.0005 | −0.596 | 0.0003 | −0.188 | 0.347 |
| 3916 | LAMP1 | lysosomal-associated membrane | 13q34 | −0.463 | 0.0002 | −0.519 | 0.0020 | −0.413 | 0.032 |
| | | protein 1 | | | | | | | |
| 3936 | LCP1 | lymphocyte cytosolic protein 1 | 13q14.3 | −0.381 | 0.0026 | −0.582 | 0.0004 | −0.123 | 0.540 |
| | | (L-plastin) | | | | | | | |
| 4285 | MIPEP | mitochondrial intermediate | 13q12 | −0.378 | 0.0029 | −0.490 | 0.0038 | −0.249 | 0.211 |
| | | peptidase | | | | | | | |
| 4752 | NEK3 | NIMA (never in mitosis gene a)- | 13q14.13 | −0.370 | 0.0036 | −0.526 | 0.0017 | −0.114 | 0.570 |
| | | related kinase 3 | | | | | | | |
| 5042 | PABPC3 | poly(A) binding protein, | 13q12-q13 | −0.372 | 0.0034 | −0.469 | 0.0059 | −0.234 | 0.240 |
| | | cytoplasmic 3 | | | | | | | |
| 5095 | PCCA | propionyl Coenzyme A | 13q32 | −0.401 | 0.0015 | −0.572 | 0.0005 | −0.164 | 0.413 |
| | | carboxylase, alpha polypeptide | | | | | | | |
| 5100 | PCDH8 | protocadherin 8 | 13q14.3-q21.1 | −0.364 | 0.0042 | −0.528 | 0.0016 | −0.104 | 0.605 |
| 5412 | UBL3 | ubiquitin-like 3 | 13q12-q13 | −0.396 | 0.0017 | −0.487 | 0.0041 | −0.272 | 0.170 |
| 6011 | GRK1 | G protein-coupled receptor kinase | 13q34 | −0.460 | 0.0002 | −0.519 | 0.0020 | −0.409 | 0.034 |
| | | 1 | | | | | | | |
| 6049 | RNF6 | ring finger protein (C3H2C3 type) | 13q12.2 | −0.365 | 0.0041 | −0.467 | 0.0062 | −0.226 | 0.256 |
| | | 6 | | | | | | | |
| 6445 | SGCG | sarcoglycan, gamma (35 kDa | 13q12 | −0.390 | 0.0021 | −0.491 | 0.0037 | −0.245 | 0.218 |
| | | dystrophin-associated | | | | | | | |
| | | glycoprotein) | | | | | | | |
| 6555 | SLC10A2 | solute carrier family 10 | 13q33 | −0.377 | 0.0030 | −0.610 | 0.0002 | −0.044 | 0.827 |
| | | (sodium/bile acid cotransporter | | | | | | | |
| | | family), member 2 | | | | | | | |
| 6564 | SLC15A1 | solute carrier family 15 | 13q33-q34 | −0.427 | 0.0007 | −0.590 | 0.0003 | −0.179 | 0.371 |
| | | (oligopeptide transporter), | | | | | | | |
| | | member 1 | | | | | | | |
| 6656 | SOX1 | SRY (sex determining region Y)- | 13q34 | −0.413 | 0.0010 | −0.546 | 0.0010 | −0.221 | 0.268 |
| | | box 1 | | | | | | | |
| 7027 | TFDP1 | transcription factor Dp-1 | 13q34 | −0.460 | 0.0002 | −0.519 | 0.0020 | −0.409 | 0.034 |
| 7178 | TPT1 | tumor protein, translationally- | 13q12-q14 | −0.394 | 0.0018 | −0.593 | 0.0003 | −0.119 | 0.553 |
| | | controlled 1 | | | | | | | |
| 7546 | ZIC2 | Zic family member 2 (odd-paired | 13q32 | −0.408 | 0.0012 | −0.574 | 0.0005 | −0.176 | 0.380 |
| | | homolog, Drosophila) | | | | | | | |
| 7750 | ZMYM2 | zinc finger, MYM-type 2 | 13q11-q12 | −0.384 | 0.0025 | −0.488 | 0.0040 | −0.244 | 0.221 |
| 8100 | IFT88 | intraflagellar transport 88 | 13q12.1 | −0.376 | 0.0030 | −0.475 | 0.0052 | −0.230 | 0.249 |
| | | homolog (Chlamydomonas) | | | | | | | |
| 8428 | STK24 | serine/threonine kinase 24 (STE20 | 13q31.2-q32.3 | −0.419 | 0.0009 | −0.615 | 0.0001 | −0.127 | 0.529 |
| | | homolog, yeast) | | | | | | | |
| 8451 | CUL4A | cullin 4A | 13q34 | −0.464 | 0.0002 | −0.519 | 0.0020 | −0.415 | 0.032 |
| 8660 | IRS2 | insulin receptor substrate 2 | 13q34 | −0.381 | 0.0027 | −0.557 | 0.0008 | −0.116 | 0.563 |
| 8803 | SUCLA2 | succinate-CoA ligase, ADP- | 13q12.2-q13.3 | −0.364 | 0.0043 | −0.559 | 0.0007 | −0.100 | 0.620 |
| | | forming, beta subunit | | | | | | | |
| 8848 | TSC22D1 | TSC22 domain family, member 1 | 13q14 | −0.368 | 0.0038 | −0.470 | 0.0057 | −0.183 | 0.362 |
| 8858 | PROZ | protein Z, vitamin K-dependent | 13q34 | −0.469 | 0.0002 | −0.519 | 0.0020 | −0.420 | 0.029 |
| | | plasma glycoprotein | | | | | | | |
| 8874 | ARHGEF7 | Rho guanine nucleotide exchange | 13q34 | −0.394 | 0.0018 | −0.542 | 0.0011 | −0.160 | 0.425 |
| | | factor (GEF) 7 | | | | | | | |
| 8881 | CDC16 | cell division cycle 16 homolog (S. | 13q34 | −0.465 | 0.0002 | −0.535 | 0.0013 | −0.402 | 0.038 |
| | | cerevisiae) | | | | | | | |
| 9071 | CLDN10 | claudin 10 | 13q31-q34 | −0.390 | 0.0021 | −0.578 | 0.0004 | −0.102 | 0.611 |
| 9107 | MTMR6 | myotubularin related protein 6 | 13q12 | −0.372 | 0.0034 | −0.467 | 0.0062 | −0.234 | 0.240 |
| 9205 | ZMYM5 | zinc finger, MYM-type 5 | 13q12 | −0.381 | 0.0027 | −0.492 | 0.0036 | −0.230 | 0.248 |
| 9365 | KL | klotho | 13q12 | −0.366 | 0.0041 | −0.450 | 0.0086 | −0.255 | 0.200 |
| 9375 | TM9SF2 | transmembrane 9 superfamily | 13q32.3 | −0.416 | 0.0009 | −0.583 | 0.0004 | −0.194 | 0.333 |
| | | member 2 | | | | | | | |
| 9445 | ITM2B | integral membrane protein 2B | 13q14.3 | −0.363 | 0.0044 | −0.562 | 0.0007 | −0.100 | 0.620 |
| 9724 | UTP14C | UTP14, U3 small nucleolar | 13q14.2 | −0.370 | 0.0036 | −0.526 | 0.0017 | −0.114 | 0.570 |
| | | ribonucleoprotein, homolog C | | | | | | | |
| | | (yeast) | | | | | | | |
| 9818 | NUPL1 | nucleoporin like 1 | 13q12.13 | −0.372 | 0.0034 | −0.467 | 0.0062 | −0.234 | 0.240 |
| 10129 | FRY | furry homolog (Drosophila) | 13q13.1 | −0.373 | 0.0034 | −0.481 | 0.0046 | −0.228 | 0.253 |
| 10160 | FARP1 | FERM, RhoGEF (ARHGEF) and | 13q32.2 | −0.415 | 0.0010 | −0.609 | 0.0002 | −0.127 | 0.529 |
| | | pleckstrin domain protein 1 | | | | | | | |
| | | (chondrocyte-derived) | | | | | | | |
| 10166 | SLC25A15 | solute carrier family 25 | 13q14 | −0.378 | 0.0029 | −0.519 | 0.0020 | −0.167 | 0.404 |
| | | (mitochondrial carrier; ornithine | | | | | | | |
| | | transporter) member 15 | | | | | | | |
| 10206 | TRIM13 | tripartite motif-containing 13 | 13q14 | −0.377 | 0.0030 | −0.544 | 0.0011 | −0.132 | 0.511 |
| 10208 | USPL1 | ubiquitin specific peptidase like 1 | 13q12-q14 | −0.375 | 0.0032 | −0.455 | 0.0078 | −0.260 | 0.190 |
| 10240 | MRPS31 | mitochondrial ribosomal protein | 13q14.11 | −0.377 | 0.0030 | −0.519 | 0.0020 | −0.168 | 0.401 |
| | | S31 | | | | | | | |
| 10257 | ABCC4 | ATP-binding cassette, sub-family | 13q32 | −0.399 | 0.0016 | −0.592 | 0.0003 | −0.129 | 0.521 |
| | | C (CFTR/MRP), member 4 | | | | | | | |
| 10284 | SAP18 | Sin3A-associated protein, 18 kDa | 13q12.11 | −0.382 | 0.0026 | −0.478 | 0.0049 | −0.240 | 0.227 |
| 10426 | TUBGCP3 | tubulin, gamma complex | 13q34 | −0.389 | 0.0021 | −0.563 | 0.0007 | −0.119 | 0.554 |
| | | associated protein 3 | | | | | | | |
| 10443 | N4BP2L2 | NEDD4 binding protein 2-like 2 | 13q13.1 | −0.372 | 0.0034 | −0.480 | 0.0047 | −0.253 | 0.203 |
| 10562 | OLFM4 | olfactomedin 4 | 13q21.1 | −0.363 | 0.0044 | −0.532 | 0.0014 | −0.104 | 0.606 |
| 10804 | GJB6 | gap junction protein, beta 6, | 13q11-q12.1\|13q12 | −0.378 | 0.0029 | −0.491 | 0.0037 | −0.222 | 0.265 |
| | | 30 kDa | | | | | | | |
| 10810 | WASF3 | WAS protein family, member 3 | 13q12 | −0.379 | 0.0028 | −0.515 | 0.0022 | −0.203 | 0.310 |
| 10910 | SUGT1 | SGT1, suppressor of G2 allele of | 13q14.3 | −0.361 | 0.0046 | −0.529 | 0.0015 | −0.098 | 0.628 |
| | | SKP1 (S. cerevisiae) | | | | | | | |
| 11061 | LECT1 | leukocyte cell derived chemotaxin | 13q14-q21 | −0.362 | 0.0045 | −0.528 | 0.0016 | −0.098 | 0.627 |
| | | 1 | | | | | | | |
| 22821 | RASA3 | RAS p21 protein activator 3 | 13q34 | −0.430 | 0.0006 | −0.519 | 0.0020 | −0.353 | 0.071 |
| 22873 | DZIP1 | DAZ interacting protein 1 | 13q32.1 | −0.379 | 0.0029 | −0.562 | 0.0007 | −0.101 | 0.617 |
| 23026 | MYO16 | myosin XVI | 13q33.3 | −0.383 | 0.0025 | −0.602 | 0.0002 | −0.080 | 0.690 |
| 23047 | PDS5B | PDS5, regulator of cohesion | 13q12.3 | −0.370 | 0.0036 | −0.466 | 0.0063 | −0.253 | 0.203 |
| | | maintenance, homolog B (S. | | | | | | | |
| | | cerevisiae) | | | | | | | |
| 23091 | ZC3H13 | zinc finger CCCH-type containing | 13q14.12 | −0.381 | 0.0026 | −0.582 | 0.0004 | −0.123 | 0.540 |
| | | 13 | | | | | | | |
| 23143 | LRCH1 | leucine-rich repeats and calponin | 13q14.13-q14.2 | −0.378 | 0.0029 | −0.577 | 0.0004 | −0.123 | 0.540 |
| | | homology (CH) domain | | | | | | | |
| | | containing 1 | | | | | | | |
| 23250 | ATP11A | ATPase, class VI, type 11A | 13q34 | −0.421 | 0.0008 | −0.519 | 0.0020 | −0.270 | 0.172 |
| 23263 | MCF2L | MCF.2 cell line derived | 13q34 | −0.416 | 0.0010 | −0.519 | 0.0020 | −0.263 | 0.185 |
| | | transforming sequence-like | | | | | | | |
| 23348 | DOCK9 | dedicator of cytokinesis 9 | 13q32.3 | −0.416 | 0.0010 | −0.584 | 0.0004 | −0.187 | 0.351 |
| 23483 | TGDS | TDP-glucose 4,6-dehydratase | 13q32.1 | −0.369 | 0.0037 | −0.574 | 0.0005 | −0.075 | 0.709 |
| 26050 | SLITRK5 | SLIT and NTRK-like family, | 13q31.2 | −0.401 | 0.0015 | −0.548 | 0.0010 | −0.211 | 0.291 |
| | | member 5 | | | | | | | |
| 26278 | SACS | spastic ataxia of Charlevoix- | 13q12 | −0.389 | 0.0021 | −0.491 | 0.0037 | −0.256 | 0.198 |
| | | Saguenay (sacsin) | | | | | | | |
| 26524 | LATS2 | LATS, large tumor suppressor, | 13q11-q12 | −0.382 | 0.0026 | −0.478 | 0.0049 | −0.240 | 0.227 |
| | | homolog 2 (Drosophila) | | | | | | | |
| 26586 | CKAP2 | cytoskeleton associated protein 2 | 13q14 | −0.371 | 0.0035 | −0.526 | 0.0017 | −0.120 | 0.552 |
| 29079 | MED4 | mediator complex subunit 4 | 13q14.2 | −0.364 | 0.0043 | −0.559 | 0.0007 | −0.100 | 0.620 |
| 51028 | VPS36 | vacuolar protein sorting 36 | 13q14.3 | −0.371 | 0.0035 | −0.526 | 0.0017 | −0.120 | 0.552 |
| | | homolog (S. cerevisiae) | | | | | | | |
| 51084 | CRYL1 | crystallin, lambda 1 | 13q12.11 | −0.376 | 0.0030 | −0.487 | 0.0040 | −0.218 | 0.274 |
| 51761 | ATP8A2 | ATPase, aminophospholipid | 13q12 | −0.366 | 0.0040 | −0.467 | 0.0062 | −0.223 | 0.263 |
| | | transporter-like, class I, type 8A, | | | | | | | |
| | | member 2 | | | | | | | |
| 53342 | IL17D | interleukin 17D | 13q12.11 | −0.376 | 0.0030 | −0.475 | 0.0052 | −0.230 | 0.249 |
| 55002 | TMCO3 | transmembrane and coiled-coil | 13q34 | −0.460 | 0.0002 | -0.519 | 0.0020 | −0.409 | 0.034 |
| | | domains 3 | | | | | | | |
| 55082 | ARGLU1 | arginine and glutamate rich 1 | 13q33.3 | −0.390 | 0.0021 | −0.611 | 0.0002 | −0.063 | 0.753 |
| 55208 | DCUN1D2 | DCN1, defective in cullin | 13q34 | −0.460 | 0.0002 | −0.519 | 0.0020 | −0.409 | 0.034 |
| | | neddylation 1 , domain containing | | | | | | | |
| | | 2 (S. cerevisiae) | | | | | | | |
| 55269 | PSPC1 | paraspeckle component 1 | 13q12.11 | −0.373 | 0.0033 | −0.486 | 0.0041 | −0.219 | 0.272 |
| 55270 | NUDT15 | nudix (nucleoside diphosphate | 13q14.2 | −0.364 | 0.0043 | −0.559 | 0.0007 | −0.100 | 0.620 |
| | | linked moiety X)-type motif 15 | | | | | | | |
| 55504 | TNFRSF19 | tumor necrosis factor receptor | 13q12.11-q12.3 | −0.397 | 0.0017 | −0.491 | 0.0037 | −0.255 | 0.199 |
| | | superfamily, member 19 | | | | | | | |
| 55608 | ANKRD10 | ankyrin repeat domain 10 | 13q34 | −0.377 | 0.0030 | −0.550 | 0.0009 | −0.113 | 0.575 |
| 55647 | RAB20 | RAB20, member RAS oncogene | 13q34 | −0.396 | 0.0017 | −0.523 | 0.0018 | −0.208 | 0.297 |
| | | family | | | | | | | |
| 55739 | CARKD | carbohydrate kinase domain | 13q34 | −0.425 | 0.0007 | −0.535 | 0.0013 | −0.260 | 0.191 |
| | | containing | | | | | | | |
| 55795 | PCID2 | PCI domain containing 2 | 13q34 | −0.464 | 0.0002 | −0.519 | 0.0020 | −0.415 | 0.032 |
| 55835 | CENPJ | centromere protein J | 13q12.12 | −0.377 | 0.0030 | −0.469 | 0.0059 | −0.248 | 0.212 |
| 55901 | THSD1 | thrombospondin, type I, domain | 13q14.3 | −0.371 | 0.0035 | −0.526 | 0.0017 | −0.120 | 0.552 |
| | | containing 1 | | | | | | | |
| 56163 | RNF17 | ring finger protein 17 | 13q12.12 | −0.379 | 0.0028 | −0.469 | 0.0059 | −0.255 | 0.199 |
| 57105 | CYSLTR2 | cysteinyl leukotriene receptor 2 | 13q14.12-q21.1 | −0.370 | 0.0036 | −0.548 | 0.0010 | −0.111 | 0.580 |
| 57213 | C13orf1 | chromosome 13 open reading | 13q14 | −0.371 | 0.0035 | −0.533 | 0.0014 | −0.132 | 0.511 |
| | | frame 1 | | | | | | | |
| 64328 | XPO4 | exportin 4 | 13q11 | −0.376 | 0.0030 | −0.475 | 0.0052 | −0.230 | 0.249 |
| 65110 | UPF3A | UPF3 regulator of nonsense | 13q34 | −0.465 | 0.0002 | −0.535 | 0.0013 | −0.402 | 0.038 |
| | | transcripts homolog A (yeast) | | | | | | | |
| 78988 | MRP63 | mitochondrial ribosomal protein | 13q12.11 | −0.382 | 0.0026 | −0.478 | 0.0049 | −0.240 | 0.227 |
| | | 63 | | | | | | | |
| 79587 | CARS2 | cysteinyl-tRNA synthetase 2, | 13q34 | −0.409 | 0.0012 | −0.535 | 0.0013 | −0.218 | 0.275 |
| | | mitochondrial (putative) | | | | | | | |
| 79621 | RNASEH2B | ribonuclease H2, subunit B | 13q14.3 | −0.371 | 0.0035 | −0.542 | 0.0011 | −0.120 | 0.552 |
| 79758 | DHRS12 | dehydrogenase/reductase (SDR | 13q14.3 | −0.370 | 0.0036 | −0.526 | 0.0017 | −0.114 | 0.570 |
| | | family) member 12 | | | | | | | |
| 79774 | GRTP1 | growth hormone regulated TBC | 13q34 | −0.449 | 0.0003 | −0.519 | 0.0020 | −0.389 | 0.045 |
| | | protein 1 | | | | | | | |
| 80183 | C13orf18 | chromosome 13 open reading | 13q14.12 | −0.381 | 0.0026 | −0.582 | 0.0004 | −0.123 | 0.540 |
| | | frame 18 | | | | | | | |
| 83446 | CCDC70 | coiled-coil domain containing 70 | 13q14.3 | −0.370 | 0.0036 | −0.526 | 0.0017 | −0.114 | 0.570 |
| 83548 | COG3 | component of oligomeric golgi | 13q14.12 | −0.395 | 0.0018 | −0.590 | 0.0003 | −0.124 | 0.537 |
| | | complex 3 | | | | | | | |
| 84056 | KATNAL1 | katanin p60 subunit A-like 1 | 13q12.3 | −0.388 | 0.0022 | −0.482 | 0.0045 | −0.260 | 0.190 |
| 84899 | TMTC4 | transmembrane and | 13q32.3 | −0.390 | 0.0021 | −0.569 | 0.0005 | −0.116 | 0.563 |
| | | tetratricopeptide repeat containing | | | | | | | |
| | | 4 | | | | | | | |
| 85416 | ZIC5 | Zic family member 5 (odd-paired | 13q32.3 | −0.408 | 0.0012 | −0.574 | 0.0005 | −0.176 | 0.380 |
| | | homolog, Drosophila) | | | | | | | |
| 87769 | A2LD1 | AIG2-like domain 1 | 13q32.3 | −0.390 | 0.0021 | −0.569 | 0.0005 | −0.116 | 0.563 |
| 90627 | STARD13 | StAR-related lipid transfer | 13q12-q13 | −0.362 | 0.0045 | −0.449 | 0.0087 | −0.248 | 0.213 |
| | | (START) domain containing 13 | | | | | | | |
| 90634 | N4BP2L1 | NEDD4 binding protein 2-like 1 | 13q12-q13 | −0.372 | 0.0034 | −0.476 | 0.0051 | −0.253 | 0.203 |
| 113622 | ADPRHL1 | ADP-ribosylhydrolase like 1 | 13q34 | −0.460 | 0.0002 | −0.519 | 0.0020 | −0.409 | 0.034 |
| 114798 | SLITRK1 | SLIT and NTRK-like family, | 13q31.1 | −0.430 | 0.0006 | −0.565 | 0.0006 | −0.294 | 0.136 |
| | | member 1 | | | | | | | |
| 115761 | ARL11 | ADP-ribosylation factor-like 11 | 13q14.3 | −0.365 | 0.0041 | −0.491 | 0.0037 | −0.164 | 0.415 |
| 115825 | WDFY2 | WD repeat and FYVE domain | 13q14.3 | −0.372 | 0.0034 | −0.528 | 0.0016 | −0.122 | 0.545 |
| | | containing 2 | | | | | | | |
| 121793 | C13orf16 | chromosome 13 open reading | 13q34 | −0.414 | 0.0010 | −0.534 | 0.0014 | −0.219 | 0.272 |
| | | frame 16 | | | | | | | |
| 122258 | C13orf28 | chromosome 13 open reading | 13q34 | −0.401 | 0.0015 | −0.563 | 0.0007 | −0.157 | 0.433 |
| | | frame 28 | | | | | | | |
| 140432 | RNF113B | ring finger protein 113B | 13q32.2 | −0.408 | 0.0012 | −0.585 | 0.0003 | −0.134 | 0.506 |
| 144983 | HNRNPA1L2 | heterogeneous nuclear | 13q14.3 | −0.362 | 0.0045 | −0.528 | 0.0016 | −0.098 | 0.628 |
| | | ribonucleoprotein A1-like 2 | | | | | | | |
| 160897 | GPR180 | G protein-coupled receptor 180 | 13q32.1 | −0.369 | 0.0037 | −0.574 | 0.0005 | −0.075 | 0.709 |
| 171425 | CLYBL | citrate lyase beta like | 13q32 | −0.414 | 0.0010 | −0.577 | 0.0004 | −0.189 | 0.345 |
| 219287 | FAM123A | family with sequence similarity | 13q12.13 | −0.372 | 0.0034 | −0.467 | 0.0062 | −0.234 | 0.240 |
| | | 123A | | | | | | | |
| 220081 | FLJ32682 | hypothetical protein FLJ32682 | 13q14.12 | −0.399 | 0.0016 | −0.582 | 0.0004 | −0.152 | 0.449 |
| 220082 | SPERT | spermatid associated | 13q14.12 | −0.397 | 0.0017 | −0.578 | 0.0004 | −0.157 | 0.435 |
| 220107 | DLEU7 | deleted in lymphocytic leukemia, 7 | 13q14.3 | −0.364 | 0.0042 | −0.540 | 0.0012 | −0.120 | 0.552 |
| 220108 | FAM124A | family with sequence similarity | 13q14.3 | −0.366 | 0.0040 | −0.506 | 0.0026 | −0.132 | 0.511 |
| | | 124A | | | | | | | |
| 220416 | LRRC63 | leucine rich repeat containing 63 | 13q14.12 | −0.381 | 0.0026 | −0.582 | 0.0004 | −0.123 | 0.540 |
| 221143 | N6AMT2 | N-6 adenine-specific DNA | 13q12.11 | −0.376 | 0.0030 | −0.475 | 0.0052 | −0.230 | 0.249 |
| | | methyltransferase 2 (putative) | | | | | | | |
| 221150 | C13orf3 | chromosome 13 open reading | 13q12.11 | −0.382 | 0.0026 | −0.478 | 0.0049 | −0.240 | 0.227 |
| | | frame 3 | | | | | | | |
| 221154 | EFHA1 | EF-hand domain family, member | 13q12.11 | −0.387 | 0.0023 | −0.480 | 0.0047 | −0.249 | 0.210 |
| | | A1 | | | | | | | |
| 221178 | SPATA13 | spermatogenesis associated 13 | 13q12.12 | −0.382 | 0.0026 | −0.477 | 0.0050 | −0.264 | 0.183 |
| 253512 | SLC25A30 | solute carrier family 25, member | 13q14.12 | −0.382 | 0.0026 | −0.573 | 0.0005 | −0.119 | 0.553 |
| | | 30 | | | | | | | |
| 253832 | ZDHHC20 | zinc finger, DHHC-type | 13q12.11 | −0.385 | 0.0024 | −0.469 | 0.0059 | −0.259 | 0.191 |
| | | containing 20 | | | | | | | |
| 259232 | NALCN | sodium leak channel, non- | 13q32.3 | −0.369 | 0.0038 | −0.573 | 0.0005 | −0.061 | 0.763 |
| | | selective | | | | | | | |
| 267012 | DAOA | D-amino acid oxidase activator | 13q33.2\|13q34 | −0.374 | 0.0032 | −0.617 | 0.0001 | −0.039 | 0.846 |
| 283489 | ZNF828 | zinc finger protein 828 | 13q34 | −0.443 | 0.0004 | −0.501 | 0.0030 | −0.402 | 0.038 |
| 283514 | SIAH3 | seven in absentia homolog 3 | 13q14.12 | −0.397 | 0.0017 | −0.578 | 0.0004 | −0.157 | 0.435 |
| | | (Drosophila) | | | | | | | |
| 283518 | KCNRG | potassium channel regulator | 13q14.3 | −0.377 | 0.0030 | −0.544 | 0.0011 | −0.132 | 0.511 |
| 337867 | UBAC2 | UBA domain containing 2 | 13q32.3 | −0.428 | 0.0006 | −0.596 | 0.0003 | −0.204 | 0.308 |
| 338872 | C1QTNF9 | C1q and tumor necrosis factor | 13q12.12 | −0.380 | 0.0027 | −0.484 | 0.0044 | −0.245 | 0.218 |
| | | related protein 9 | | | | | | | |
| 341676 | NEK5 | NIMA (never in mitosis gene a)- | 13q14.3 | −0.370 | 0.0036 | −0.526 | 0.0017 | −0.114 | 0.570 |
| | | related kinase 5 | | | | | | | |
| 348013 | FAM70B | family with sequence similarity | 13q34 | −0.449 | 0.0003 | −0.519 | 0.0020 | −0.396 | 0.041 |
| | | 70, member B | | | | | | | |
| 386618 | KCTD4 | potassium channel tetramerisation | 13q14.12 | −0.387 | 0.0023 | −0.577 | 0.0004 | −0.122 | 0.545 |
| | | domain containing 4 | | | | | | | |
| 387911 | RP11-45B20.2 | collagen triple helix repeat- | 13q12.12 | −0.370 | 0.0036 | −0.485 | 0.0043 | −0.245 | 0.217 |
| | | containing | | | | | | | |
| 387914 | SHISA2 | shisa homolog 2 (Xenopus laevis) | 13q12.13 | −0.360 | 0.0047 | −0.467 | 0.0062 | −0.212 | 0.289 |
| 387923 | SERP2 | stress-associated endoplasmic | 13q14.11 | −0.386 | 0.0024 | −0.470 | 0.0057 | −0.233 | 0.242 |
| | | reticulum protein family member | | | | | | | |
| | | 2 | | | | | | | |
| 390424 | LOC390424 | similar to hCG1639781 | 13q33.3 | −0.393 | 0.0019 | −0.615 | 0.0001 | −0.063 | 0.753 |
| 400110 | FLJ46358 | FLJ46358 protein | 13q12.12 | −0.374 | 0.0033 | −0.486 | 0.0041 | −0.250 | 0.208 |
| 400165 | C13orf35 | chromosome 13 open reading | 13q34 | −0.391 | 0.0020 | −0.531 | 0.0015 | −0.176 | 0.379 |
| | | frame 35 | | | | | | | |
| 440138 | ALG11 | asparagine-linked glycosylation | 13q14.2 | −0.370 | 0.0036 | −0.526 | 0.0017 | −0.114 | 0.570 |
| | | 11, alpha-1,2-mannosyltransferase | | | | | | | |
| | | homolog (yeast) | | | | | | | |
| 445341 | TNAP | TRAFs and NIK-associated | Chr13 | −0.367 | 0.0040 | −0.464 | 0.0065 | −0.205 | 0.305 |
| | | protein | | | | | | | |
| 542767 | PCOTH | prostate collagen triple helix | 13q12 | −0.370 | 0.0036 | −0.485 | 0.0043 | −0.245 | 0.217 |
| 646357 | LOC646357 | hypothetical LOC646357 | 13q12.12 | −0.380 | 0.0027 | −0.484 | 0.0044 | −0.245 | 0.218 |
| 646799 | RP11-37E23.4 | ZAR1-like protein | 13q13.1 | −0.405 | 0.0013 | −0.491 | 0.0037 | −0.294 | 0.136 |
| 647166 | LOC647166 | similar to hCG28707 | 13q14.3 | −0.368 | 0.0038 | −0.511 | 0.0024 | −0.132 | 0.511 |
| 650794 | LOC650794 | similar to FRAS1 related | 13q12.11 | −0.379 | 0.0028 | −0.472 | 0.0055 | −0.240 | 0.227 |
| | | extracellular matrix protein 2 | | | | | | | |
| 728767 | LOC728767 | hypothetical LOC728767 | 13q34 | −0.395 | 0.0018 | −0.584 | 0.0004 | −0.115 | 0.569 |
| 731932 | LOC731932 | hypothetical LOC731932 | 13q14.13 | −0.381 | 0.0026 | −0.582 | 0.0004 | −0.123 | 0.540 |
| 100128430 | LOC100128430 | hypothetical protein | 13q34 | −0.463 | 0.0002 | −0.519 | 0.0020 | −0.424 | 0.027 |
| | | LOC100128430 | | | | | | | |
| 100128765 | LOC100128765 | similar to hCG2041516 | 13q12.11 | −0.379 | 0.0028 | −0.432 | 0.0121 | −0.292 | 0.139 |
| 100129122 | LOC100129122 | hypothetical protein | 13q32.3 | −0.405 | 0.0013 | −0.577 | 0.0004 | −0.183 | 0.360 |
| | | LOC100129122 | | | | | | | |
| 100129174 | RP11-90M2.3 | novel protein similar to | 13q14.2 | −0.364 | 0.0043 | −0.559 | 0.0007 | −0.100 | 0.620 |
| | | polymerase (RNA) II (DNA | | | | | | | |
| | | directed) polypeptide K, 7.0 kDa | | | | | | | |
| | | POLR2K | | | | | | | |
| 100129303 | LOC100129303 | hypothetical protein | 13q14.3 | −0.366 | 0.0040 | −0.506 | 0.0026 | −0.132 | 0.511 |
| | | LOC100129303 | | | | | | | |
| 100129390 | LOC100129390 | hypothetical protein | 13q34 | −0.377 | 0.0030 | −0.550 | 0.0009 | −0.113 | 0.575 |
| | | LOC100129390 | | | | | | | |
| 100129538 | LOC100129538 | hypothetical protein | 13q32.1 | −0.369 | 0.0037 | −0.574 | 0.0005 | −0.075 | 0.709 |
| | | LOC100129538 | | | | | | | |
| 100129597 | LOC100129597 | hypothetical protein | 13q14.2 | −0.370 | 0.0036 | −0.548 | 0.0010 | −0.111 | 0.580 |
| | | LOC100129597 | | | | | | | |
| 100129836 | LOC100129836 | hypothetical protein | 13q34 | −0.389 | 0.0021 | −0.498 | 0.0032 | −0.221 | 0.269 |
| | | LOC100129836 | | | | | | | |
| 100130463 | LOC100130463 | hypothetical protein | 13q34 | −0.465 | 0.0002 | −0.535 | 0.0013 | −0.402 | 0.038 |
| | | LOC100130463 | | | | | | | |
| 100130563 | LOC100130563 | similar to CNOT4 protein | 13q12.11 | −0.382 | 0.0026 | −0.478 | 0.0049 | −0.240 | 0.227 |
| 100130779 | LOC100130779 | hypothetical protein | 13q14.11 | −0.386 | 0.0024 | −0.470 | 0.0057 | −0.233 | 0.242 |
| | | LOC100130779 | | | | | | | |
| 100130979 | LOC100130979 | hypothetical protein | 13q12.11 | −0.376 | 0.0030 | −0.475 | 0.0052 | −0.230 | 0.249 |
| | | LOC100130979 | | | | | | | |
| 100131110 | LOC100131110 | hypothetical protein | 13q32.3 | −0.411 | 0.0011 | −0.569 | 0.0006 | −0.204 | 0.308 |
| | | LOC100131110 | | | | | | | |
| 100131435 | LOC100131435 | hypothetical protein | 13q34 | −0.397 | 0.0017 | −0.535 | 0.0013 | −0.183 | 0.361 |
| | | LOC100131435 | | | | | | | |
| 100131766 | OK/SW-CL.58 | OK/SW-CL.58 | 13q12.3 | −0.381 | 0.0027 | −0.511 | 0.0024 | −0.232 | 0.245 |
| 100131993 | LOC100131993 | similar to hCG2020760 | 13q14.2 | −0.370 | 0.0036 | −0.548 | 0.0010 | −0.111 | 0.580 |
| 100132099 | UNQ1829 | FRSS1829 | 13q32.3 | −0.405 | 0.0013 | −0.577 | 0.0004 | −0.183 | 0.360 |
| 100132761 | LOC100132761 | hypothetical protein | 13q14.3 | −0.362 | 0.0045 | −0.528 | 0.0016 | −0.098 | 0.628 |
| | | LOC100132761 | | | | | | | |
| 100133284 | LOC100133284 | similar to hCG1791648 | 13q12.13 | −0.372 | 0.0034 | −0.469 | 0.0059 | −0.234 | 0.240 |

TABLE 5B

| | | | | T-test | | |
|---|---|---|---|---|---|---|
| | | | | | in KRAS-MUT | in KRAS-WT |
| | | | | in all lines | only | only |
| | | | | (n = 60) | (n = 33) | (n = 27) |
| | | | | p-value, | p-value, | p-value, |
| Locus | Symbol | Description | Band | S vs. R | S vs. R | S vs. R |
| 143 | PARP4 | poly (ADP-ribose) polymerase | 13q11 | 0.0034 | 0.0040 | 0.1759 |
| | | family, member 4 | | | | |
| 241 | ALOX5AP | arachidonate 5-lipoxygenase- | 13q12 | 0.0039 | 0.0073 | 0.1355 |
| | | activating protein | | | | |
| 479 | ATP12A | ATPase, H+/K+ transporting, | 13q12.12\|13q12.1- | 0.0040 | 0.0050 | 0.1774 |
| | | nongastric, alpha polypeptide | q12.3 | | | |
| 496 | ATP4B | ATPase, H+/K+ exchanging, beta | 13q34 | 0.0003 | 0.0036 | 0.0157 |
| | | polypeptide | | | | |
| 540 | ATP7B | ATPase, Cu++ transporting, beta | 13q14.3 | 0.0005 | 0.0001 | 0.3162 |
| | | polypeptide | | | | |
| 675 | BRCA2 | breast cancer 2, early onset | 13q12.3 | 0.0022 | 0.0065 | 0.0873 |
| 1024 | CDK8 | cyclin-dependent kinase 8 | 13q12 | 0.0022 | 0.0024 | 0.1639 |
| 1102 | RCBTB2 | regulator of chromosome | 13q14.3 | 0.0017 | 0.0006 | 0.2805 |
| | | condensation (RCC1) and BTB | | | | |
| | | (POZ) domain containing protein 2 | | | | |
| 1282 | COL4A1 | collagen, type IV, alpha 1 | 13q34 | 0.0020 | 0.0008 | 0.2765 |
| 1284 | COL4A2 | collagen, type IV, alpha 2 | 13q34 | 0.0021 | 0.0014 | 0.2064 |
| 1361 | CPB2 | carboxypeptidase B2 (plasma) | 13q14.11 | 0.0009 | 0.0005 | 0.1796 |
| 1638 | DCT | dopachrome tautomerase | 13q32 | 0.0048 | 0.0012 | 0.4102 |
| | | (dopachrome delta-isomerase, | | | | |
| | | tyrosine-related protein 2) | | | | |
| 1880 | GPR183 | G protein-coupled receptor 183 | 13q32.3 | 0.0007 | 0.0008 | 0.1120 |
| 1948 | EFNB2 | ephrin-B2 | 13q33 | 0.0024 | 0.0003 | 0.4897 |
| 2073 | ERCC5 | excision repair cross- | 13q33 | 0.0043 | 0.0004 | 0.5811 |
| | | complementing rodent repair | | | | |
| | | deficiency, complementation group | | | | |
| | | 5 | | | | |
| 2098 | ESD | esterase D/formylglutathione | 13q14.1-q14.2 | 0.0017 | 0.0013 | 0.1856 |
| | | hydrolase | | | | |
| 2155 | F7 | coagulation factor VII (serum | 13q34 | 0.0015 | 0.0028 | 0.1037 |
| | | prothrombin conversion accelerator) | | | | |
| 2159 | F10 | coagulation factor X | 13q34 | 0.0015 | 0.0028 | 0.1037 |
| 2254 | FGF9 | fibroblast growth factor 9 (glia- | 13q11-q12 | 0.0019 | 0.0014 | 0.1948 |
| | | activating factor) | | | | |
| 2308 | FOXO1 | forkhead box O1 | 13q14.1 | 0.0010 | 0.0002 | 0.3006 |
| 2621 | GAS6 | growth arrest-specific 6 | 13q34 | 0.0003 | 0.0037 | 0.0194 |
| 2700 | GJA3 | gap junction protein, alpha 3, | 13q11-q12 | 0.0023 | 0.0026 | 0.1627 |
| | | 46 kDa | | | | |
| 2706 | GJB2 | gap junction protein, beta 2, 26 kDa | 13q11-q12 | 0.0022 | 0.0024 | 0.1642 |
| 2835 | GPR12 | G protein-coupled receptor 12 | 13q12 | 0.0047 | 0.0029 | 0.2730 |
| 2841 | GPR18 | G protein-coupled receptor 18 | 13q32 | 0.0007 | 0.0008 | 0.1120 |
| 2963 | GTF2F2 | general transcription factor IIF, | 13q14 | 0.0024 | 0.0011 | 0.2692 |
| | | polypeptide 2, 30 kDa | | | | |
| 3146 | HMGB1 | high-mobility group box 1 | 13q12 | 0.0030 | 0.0055 | 0.1304 |
| 3356 | HTR2A | 5-hydroxytryptamine (serotonin) | 13q14-q21 | 0.0017 | 0.0013 | 0.1856 |
| | | receptor 2A | | | | |
| 3621 | ING1 | inhibitor of growth family, member | 13q34 | 0.0022 | 0.0015 | 0.2119 |
| | | 1 | | | | |
| 3843 | IPO5 | importin 5 | 13q32.2 | 0.0003 | 0.0001 | 0.1882 |
| 3916 | LAMP1 | lysosomal-associated membrane | 13q34 | 0.0002 | 0.0036 | 0.0124 |
| | | protein 1 | | | | |
| 3936 | LCP1 | lymphocyte cytosolic protein 1 (L- | 13q14.3 | 0.0009 | 0.0005 | 0.1796 |
| | | plastin) | | | | |
| 4285 | MIPEP | mitochondrial intermediate | 13q12 | 0.0031 | 0.0064 | 0.1195 |
| | | peptidase | | | | |
| 4752 | NEK3 | NIMA (never in mitosis gene a)- | 13q14.13 | 0.0005 | 0.0001 | 0.3162 |
| | | related kinase 3 | | | | |
| 5042 | PABPC3 | poly(A) binding protein, | 13q12-q13 | 0.0041 | 0.0048 | 0.1859 |
| | | cytoplasmic 3 | | | | |
| 5095 | PCCA | propionyl Coenzyme A | 13q32 | 0.0009 | 0.0004 | 0.2100 |
| | | carboxylase, alpha polypeptide | | | | |
| 5100 | PCDH8 | protocadherin 8 | 13q14.3-q21.1 | 0.0005 | 0.0001 | 0.3288 |
| 5412 | UBL3 | ubiquitin-like 3 | 13q12-q13 | 0.0020 | 0.0039 | 0.1116 |
| 6011 | GRK1 | G protein-coupled receptor kinase 1 | 13q34 | 0.0003 | 0.0036 | 0.0157 |
| 6049 | RNF6 | ring finger protein (C3H2C3 type) 6 | 13q12.2 | 0.0032 | 0.0032 | 0.1923 |
| 6445 | SGCG | sarcoglycan, gamma (35 kDa | 13q12 | 0.0020 | 0.0023 | 0.1565 |
| | | dystrophin-associated glycoprotein) | | | | |
| 6555 | SLC10A2 | solute carrier family 10 | 13q33 | 0.0043 | 0.0004 | 0.5811 |
| | | (sodium/bile acid cotransporter | | | | |
| | | family), member 2 | | | | |
| 6564 | SLC15A1 | solute carrier family 15 | 13q33-q34 | 0.0003 | 0.0002 | 0.1289 |
| | | (oligopeptide transporter), member | | | | |
| | | 1 | | | | |
| 6656 | SOX1 | SRY (sex determining region Y)- | 13q34 | 0.0013 | 0.0025 | 0.1003 |
| | | box 1 | | | | |
| 7027 | TFDP1 | transcription factor Dp-1 | 13q34 | 0.0003 | 0.0036 | 0.0157 |
| 7178 | TPT1 | tumor protein, translationally- | 13q12-q14 | 0.0027 | 0.0014 | 0.2602 |
| | | controlled 1 | | | | |
| 7546 | ZIC2 | Zic family member 2 (odd-paired | 13q32 | 0.0006 | 0.0003 | 0.1824 |
| | | homolog, Drosophila) | | | | |
| 7750 | ZMYM2 | zinc finger, MYM-type 2 | 13q11-q12 | 0.0017 | 0.0030 | 0.1110 |
| 8100 | IFT88 | intraflagellar transport 88 homolog | 13q12.1 | 0.0017 | 0.0014 | 0.1803 |
| | | (Chlamydomonas) | | | | |
| 8428 | STK24 | serine/threonine kinase 24 (STE20 | 13q31.2-q32.3 | 0.0003 | 0.0001 | 0.1996 |
| | | homolog, yeast) | | | | |
| 8451 | CUL4A | cullin 4A | 13q34 | 0.0002 | 0.0036 | 0.0111 |
| 8660 | IRS2 | insulin receptor substrate 2 | 13q34 | 0.0032 | 0.0010 | 0.3385 |
| 8803 | SUCLA2 | succinate-CoA ligase, ADP- | 13q12.2-q13.3 | 0.0024 | 0.0009 | 0.2868 |
| | | forming, beta subunit | | | | |
| 8848 | TSC22D1 | TSC22 domain family, member 1 | 13q14 | 0.0008 | 0.0003 | 0.2292 |
| 8858 | PROZ | protein Z, vitamin K-dependent | 13q34 | 0.0002 | 0.0032 | 0.0141 |
| | | plasma glycoprotein | | | | |
| 8874 | ARHGEF7 | Rho guanine nucleotide exchange | 13q34 | 0.0038 | 0.0025 | 0.2492 |
| | | factor (GEF) 7 | | | | |
| 8881 | CDC16 | cell division cycle 16 homolog | 13q34 | 0.0002 | 0.0036 | 0.0120 |
| | | (S. cerevisiae) | | | | |
| 9071 | CLDN10 | claudin 10 | 13q31-q34 | 0.0020 | 0.0003 | 0.3934 |
| 9107 | MTMR6 | myotubularin related protein 6 | 13q12 | 0.0023 | 0.0022 | 0.1781 |
| 9205 | ZMYM5 | zinc finger, MYM-type 5 | 13q12 | 0.0013 | 0.0018 | 0.1219 |
| 9365 | KL | klotho | 13q12 | 0.0022 | 0.0056 | 0.0963 |
| 9375 | TM9SF2 | transmembrane 9 superfamily | 13q32.3 | 0.0006 | 0.0005 | 0.1414 |
| | | member 2 | | | | |
| 9445 | ITM2B | integral membrane protein 2B | 13q14.3 | 0.0029 | 0.0012 | 0.2874 |
| 9724 | UTP14C | UTP14, U3 small nucleolar | 13q14.2 | 0.0005 | 0.0001 | 0.3162 |
| | | ribonucleoprotein, homolog C | | | | |
| | | (yeast) | | | | |
| 9818 | NUPL1 | nucleoporin like 1 | 13q12.13 | 0.0023 | 0.0022 | 0.1781 |
| 10129 | FRY | furry homolog (Drosophila) | 13q13.1 | 0.0037 | 0.0051 | 0.1621 |
| 10160 | FARP1 | FERM, RhoGEF (ARHGEF) and | 13q32.2 | 0.0004 | 0.0001 | 0.2015 |
| | | pleckstrin domain protein 1 | | | | |
| | | (chondrocyte-derived) | | | | |
| 10166 | SLC25A15 | solute carrier family 25 | 13q14 | 0.0011 | 0.0003 | 0.2796 |
| | | (mitochondrial carrier; ornithine | | | | |
| | | transporter) member 15 | | | | |
| 10206 | TRIM 13 | tripartite motif-containing 13 | 13q14 | 0.0006 | 0.0002 | 0.2173 |
| 10208 | USPL1 | ubiquitin specific peptidase like 1 | 13q12-q14 | 0.0030 | 0.0055 | 0.1304 |
| 10240 | MRPS31 | mitochondrial ribosomal protein | 13q14.11 | 0.0010 | 0.0003 | 0.2658 |
| | | S31 | | | | |
| 10257 | ABCC4 | ATP-binding cassette, sub-family C | 13q32 | 0.0015 | 0.0006 | 0.2553 |
| | | (CFTR/MRP), member 4 | | | | |
| 10284 | SAP18 | Sin3A-associated protein, 18 kDa | 13q12.11 | 0.0016 | 0.0014 | 0.1662 |
| 10426 | TUBGCP3 | tubulin, gamma complex associated | 13q34 | 0.0025 | 0.0011 | 0.2657 |
| | | protein 3 | | | | |
| 10443 | N4BP2L2 | NEDD4 binding protein 2-like 2 | 13q13.1 | 0.0025 | 0.0096 | 0.0746 |
| 10562 | OLFM4 | olfactomedin 4 | 13q21.1 | 0.0005 | 0.0001 | 0.3433 |
| 10804 | GJB6 | gap junction protein, beta 6, 30 kDa | 13q11- | 0.0022 | 0.0024 | 0.1642 |
| | | | q12.1\|13q12 | | | |
| 10810 | WASF3 | WAS protein family, member 3 | 13q12 | 0.0036 | 0.0020 | 0.2675 |
| 10910 | SUGT1 | SGT1, suppressor of G2 allele of | 13q14.3 | 0.0008 | 0.0001 | 0.3939 |
| | | SKP1 (S. cerevisiae) | | | | |
| 11061 | LECT1 | leukocyte cell derived chemotaxin 1 | 13q14-q21 | 0.0008 | 0.0001 | 0.3953 |
| 22821 | RASA3 | RAS p21 protein activator 3 | 13q34 | 0.0007 | 0.0040 | 0.0391 |
| 22873 | DZIP1 | DAZ interacting protein 1 | 13q32.1 | 0.0041 | 0.0009 | 0.4276 |
| 23026 | MYO16 | myosin XVI | 13q33.3 | 0.0049 | 0.0008 | 0.4933 |
| 23047 | PDS5B | PDS5, regulator of cohesion | 13q12.3 | 0.0024 | 0.0077 | 0.0819 |
| | | maintenance, homolog B | | | | |
| | | (S. cerevisiae) | | | | |
| 23091 | ZC3H13 | zinc finger CCCH-type containing | 13q14.12 | 0.0009 | 0.0005 | 0.1796 |
| | | 13 | | | | |
| 23143 | LRCH1 | leucine-rich repeats and calponin | 13q14.13-q14.2 | 0.0012 | 0.0009 | 0.1808 |
| | | homology (CH) domain containing | | | | |
| | | 1 | | | | |
| 23250 | ATP11A | ATPase, class VI, type 11A | 13q34 | 0.0016 | 0.0028 | 0.1130 |
| 23263 | MCF2L | MCF.2 cell line derived | 13q34 | 0.0015 | 0.0028 | 0.1037 |
| | | transforming sequence-like | | | | |
| 23348 | DOCK9 | dedicator of cytokinesis 9 | 13q32.3 | 0.0006 | 0.0006 | 0.1176 |
| 23483 | TGDS | TDP-glucose 4,6-dehydratase | 13q32.1 | 0.0048 | 0.0012 | 0.4102 |
| 26050 | SLITRK5 | SLIT and NTRK-like family, | 13q31.2 | 0.0006 | 0.0007 | 0.1051 |
| | | member 5 | | | | |
| 26278 | SACS | spastic ataxia of Charlevoix- | 13q12 | 0.0021 | 0.0038 | 0.1193 |
| | | Saguenay (sacsin) | | | | |
| 26524 | LATS2 | LATS, large tumor suppressor, | 13q11-q12 | 0.0016 | 0.0014 | 0.1662 |
| | | homolog 2 (Drosophila) | | | | |
| 26586 | CKAP2 | cytoskeleton associated protein 2 | 13q14 | 0.0003 | 0.0001 | 0.2672 |
| 29079 | MED4 | mediator complex subunit 4 | 13q14.2 | 0.0024 | 0.0009 | 0.2868 |
| 51028 | VPS36 | vacuolar protein sorting 36 homolog | 13q14.3 | 0.0003 | 0.0001 | 0.2672 |
| | | (S. cerevisiae) | | | | |
| 51084 | CRYL1 | crystallin, lambda 1 | 13q12.11 | 0.0020 | 0.0016 | 0.1877 |
| 51761 | ATP8A2 | ATPase, aminophospholipid | 13q12 | 0.0029 | 0.0024 | 0.2039 |
| | | transporter-like, class I, type 8A, | | | | |
| | | member 2 | | | | |
| 53342 | IL17D | interleukin 17D | 13q12.11 | 0.0017 | 0.0014 | 0.1803 |
| 55002 | TMCO3 | transmembrane and coiled-coil | 13q34 | 0.0003 | 0.0036 | 0.0157 |
| | | domains 3 | | | | |
| 55082 | ARGLU1 | arginine and glutamate rich 1 | 13q33.3 | 0.0024 | 0.0003 | 0.4897 |
| 55208 | DCUN1D2 | DCN1, defective in cullin | 13q34 | 0.0003 | 0.0036 | 0.0157 |
| | | neddylation 1, domain containing 2 | | | | |
| | | (S. cerevisiae) | | | | |
| 55269 | PSPC1 | paraspeckle component 1 | 13q12.11 | 0.0024 | 0.0026 | 0.1677 |
| 55270 | NUDT15 | nudix (nucleoside diphosphate | 13q14.2 | 0.0024 | 0.0009 | 0.2868 |
| | | linked moiety X)-type motif 15 | | | | |
| 55504 | TNFRSF19 | tumor necrosis factor receptor | 13q12.11-q12.3 | 0.0014 | 0.0018 | 0.1339 |
| | | superfamily, member 19 | | | | |
| 55608 | ANKRD10 | ankyrin repeat domain 10 | 13q34 | 0.0047 | 0.0020 | 0.3244 |
| 55647 | RAB20 | RAB20, member RAS oncogene | 13q34 | 0.0016 | 0.0016 | 0.1521 |
| | | family | | | | |
| 55739 | CARKD | carbohydrate kinase domain | 13q34 | 0.0006 | 0.0010 | 0.0944 |
| | | containing | | | | |
| 55795 | PCID2 | PCI domain containing 2 | 13q34 | 0.0002 | 0.0036 | 0.0111 |
| 55835 | CENPJ | centromere protein J | 13q12.12 | 0.0036 | 0.0055 | 0.1523 |
| 55901 | THSD1 | thrombospondin, type I, domain | 13q14.3 | 0.0003 | 0.0001 | 0.2672 |
| | | containing 1 | | | | |
| 56163 | RNF17 | ring finger protein 17 | 13q12.12 | 0.0035 | 0.0056 | 0.1449 |
| 57105 | CYSLTR2 | cysteinyl leukotriene receptor 2 | 13q14.12-q21.1 | 0.0017 | 0.0006 | 0.2805 |
| 57213 | C13orf1 | chromosome 13 open reading frame | 13q14 | 0.0008 | 0.0003 | 0.2208 |
| | | 1 | | | | |
| 64328 | XPO4 | exportin 4 | 13q11 | 0.0017 | 0.0014 | 0.1803 |
| 65110 | UPF3A | UPF3 regulator of nonsense | 13q34 | 0.0002 | 0.0036 | 0.0120 |
| | | transcripts homolog A (yeast) | | | | |
| 78988 | MRP63 | mitochondrial ribosomal protein 63 | 13q12.11 | 0.0016 | 0.0014 | 0.1662 |
| 79587 | CARS2 | cysteinyl-tRNA synthetase 2, | 13q34 | 0.0011 | 0.0013 | 0.1370 |
| | | mitochondrial (putative) | | | | |
| 79621 | RNASEH2B | ribonuclease H2, subunit B | 13q14.3 | 0.0006 | 0.0001 | 0.2878 |
| 79758 | DHRS12 | dehydrogenase/reductase (SDR | 13q14.3 | 0.0005 | 0.0001 | 0.3162 |
| | | family) member 12 | | | | |
| 79774 | GRTP1 | growth hormone regulated TBC | 13q34 | 0.0005 | 0.0040 | 0.0293 |
| | | protein 1 | | | | |
| 80183 | C13orf18 | chromosome 13 open reading frame | 13q14.12 | 0.0009 | 0.0005 | 0.1796 |
| | | 18 | | | | |
| 83446 | CCDC70 | coiled-coil domain containing 70 | 13q14.3 | 0.0005 | 0.0001 | 0.3162 |
| 83548 | COG3 | component of oligomeric golgi | 13q14.12 | 0.0012 | 0.0004 | 0.2691 |
| | | complex 3 | | | | |
| 84056 | KATNAL1 | katanin p60 subunit A-like 1 | 13q12.3 | 0.0024 | 0.0038 | 0.1299 |
| 84899 | TMTC4 | transmembrane and | 13q32.3 | 0.0013 | 0.0003 | 0.3161 |
| | | tetratricopeptide repeat containing 4 | | | | |
| 85416 | ZIC5 | Zic family member 5 (odd-paired | 13q32.3 | 0.0006 | 0.0003 | 0.1824 |
| | | homolog, Drosophila) | | | | |
| 87769 | A2LD1 | AIG2-like domain 1 | 13q32.3 | 0.0013 | 0.0003 | 0.3161 |
| 90627 | STARD13 | StAR-related lipid transfer | 13q12-q13 | 0.0025 | 0.0061 | 0.1025 |
| | | (START) domain containing 13 | | | | |
| 90634 | N4BP2L1 | NEDD4 binding protein 2-like 1 | 13q12-q13 | 0.0025 | 0.0090 | 0.0763 |
| 113622 | ADPRHL1 | ADP-ribosylhydrolase like 1 | 13q34 | 0.0003 | 0.0036 | 0.0157 |
| 114798 | SLITRK1 | SLIT and NTRK-like family, | 13q31.1 | 0.0003 | 0.0008 | 0.0535 |
| | | member 1 | | | | |
| 115761 | ARL11 | ADP-ribosylation factor-like 11 | 13q14.3 | 0.0008 | 0.0011 | 0.1136 |
| 115825 | WDFY2 | WD repeat and FYVE domain | 13q14.3 | 0.0005 | 0.0001 | 0.2958 |
| | | containing 2 | | | | |
| 121793 | C13orf16 | chromosome 13 open reading frame | 13q34 | 0.0027 | 0.0037 | 0.1481 |
| | | 16 | | | | |
| 122258 | C13orf28 | chromosome 13 open reading frame | 13q34 | 0.0014 | 0.0011 | 0.1814 |
| | | 28 | | | | |
| 140432 | RNF113B | ring finger protein 113B | 13q32.2 | 0.0005 | 0.0002 | 0.2169 |
| 144983 | HNRNPA1L2 | heterogeneous nuclear | 13q14.3 | 0.0008 | 0.0001 | 0.4005 |
| | | ribonucleoprotein A1-like 2 | | | | |
| 160897 | GPR180 | G protein-coupled receptor 180 | 13q32.1 | 0.0048 | 0.0012 | 0.4102 |
| 171425 | CLYBL | citrate lyase beta like | 13q32 | 0.0006 | 0.0004 | 0.1714 |
| 219287 | FAM123A | family with sequence similarity | 13q12.13 | 0.0023 | 0.0022 | 0.1781 |
| | | 123A | | | | |
| 220081 | FLJ32682 | hypothetical protein FLJ32682 | 13q14.12 | 0.0005 | 0.0004 | 0.1344 |
| 220082 | SPERT | spermatid associated | 13q14.12 | 0.0005 | 0.0005 | 0.1133 |
| 220107 | DLEU7 | deleted in lymphocytic leukemia, 7 | 13q14.3 | 0.0013 | 0.0004 | 0.2732 |
| 220108 | FAM124A | family with sequence similarity | 13q14.3 | 0.0004 | 0.0001 | 0.2743 |
| | | 124A | | | | |
| 220416 | LRRC63 | leucine rich repeat containing 63 | 13q14.12 | 0.0009 | 0.0005 | 0.1796 |
| 221143 | N6AMT2 | N-6 adenine-specific DNA | 13q12.11 | 0.0017 | 0.0014 | 0.1803 |
| | | methyltransferase 2 (putative) | | | | |
| 221150 | C13orf3 | chromosome 13 open reading frame | 13q12.11 | 0.0016 | 0.0014 | 0.1662 |
| | | 3 | | | | |
| 221154 | EFHA1 | EF-hand domain family, member | 13q12.11 | 0.0014 | 0.0014 | 0.1495 |
| | | A1 | | | | |
| 221178 | SPATA13 | spermatogenesis associated 13 | 13q12.12 | 0.0033 | 0.0057 | 0.1356 |
| 253512 | SLC25A30 | solute carrier family 25, member 30 | 13q14.12 | 0.0022 | 0.0010 | 0.2582 |
| 253832 | ZDHHC20 | zinc finger, DHHC-type containing | 13q12.11 | 0.0015 | 0.0019 | 0.1403 |
| | | 20 | | | | |
| 259232 | NALCN | sodium leak channel, non-selective | 13q32.3 | 0.0029 | 0.0004 | 0.4656 |
| 267012 | DAOA | D-amino acid oxidase activator | 13q33.2\|13q34 | 0.0045 | 0.0004 | 0.5980 |
| 283489 | ZNF828 | zinc finger protein 828 | 13q34 | 0.0003 | 0.0052 | 0.0118 |
| 283514 | SIAH3 | seven in absentia homolog 3 | 13q14.12 | 0.0005 | 0.0005 | 0.1133 |
| | | (Drosophila) | | | | |
| 283518 | KCNRG | potassium channel regulator | 13q14.3 | 0.0006 | 0.0002 | 0.2173 |
| 337867 | UBAC2 | UBA domain containing 2 | 13q32.3 | 0.0003 | 0.0004 | 0.0854 |
| 338872 | C1QTNF9 | C1q and tumor necrosis factor | 13q12.12 | 0.0041 | 0.0047 | 0.1878 |
| | | related protein 9 | | | | |
| 341676 | NEK5 | NIMA (never in mitosis gene a)- | 13q14.3 | 0.0005 | 0.0001 | 0.3162 |
| | | related kinase 5 | | | | |
| 348013 | FAM70B | family with sequence similarity 70, | 13q34 | 0.0004 | 0.0041 | 0.0222 |
| | | member B | | | | |
| 386618 | KCTD4 | potassium channel tetramerisation | 13q14.12 | 0.0019 | 0.0008 | 0.2693 |
| | | domain containing 4 | | | | |
| 387911 | RP11-45B20.2 | collagen triple helix repeat- | 13q12.12 | 0.0039 | 0.0082 | 0.1248 |
| | | containing | | | | |
| 387914 | SHISA2 | shisa homolog 2 (Xenopus laevis) | 13q12.13 | 0.0037 | 0.0027 | 0.2329 |
| 387923 | SERP2 | stress-associated endoplasmic | 13q14.11 | 0.0005 | 0.0005 | 0.1241 |
| | | reticulum protein family member 2 | | | | |
| 390424 | LOC390424 | similar to hCG1639781 | 13q33.3 | 0.0024 | 0.0003 | 0.4913 |
| 400110 | FLJ46358 | FLJ46358 protein | 13q12.12 | 0.0031 | 0.0067 | 0.1171 |
| 400165 | C13orf35 | chromosome 13 open reading frame | 13q34 | 0.0033 | 0.0021 | 0.2445 |
| | | 35 | | | | |
| 440138 | ALG11 | asparagine-linked glycosylation 11, | 13q14.2 | 0.0005 | 0.0001 | 0.3162 |
| | | alpha-1,2-mannosyltransferase | | | | |
| | | homolog (yeast) | | | | |
| 445341 | TNAP | TRAFs and NIK-associated protein | Chr13 | 0.0020 | 0.0020 | 0.1676 |
| 542767 | PCOTH | prostate collagen triple helix | 13q12 | 0.0039 | 0.0082 | 0.1248 |
| 646357 | LOC646357 | hypothetical LOC646357 | 13q12.12 | 0.0041 | 0.0047 | 0.1878 |
| 646799 | RP11-37E23.4 | ZAR1-like protein | 13q13.1 | 0.0020 | 0.0053 | 0.0892 |
| 647166 | LOC647166 | similar to hCG28707 | 13q14.3 | 0.0004 | 0.0001 | 0.2725 |
| 650794 | LOC650794 | similar to FRAS1 related | 13q12.11 | 0.0018 | 0.0017 | 0.1677 |
| | | extracellular matrix protein 2 | | | | |
| 728767 | LOC728767 | hypothetical LOC728767 | 13q34 | 0.0020 | 0.0005 | 0.3464 |
| 731932 | LOC731932 | hypothetical LOC731932 | 13q14.13 | 0.0009 | 0.0005 | 0.1796 |
| 100128430 | LOC100128430 | hypothetical protein | 13q34 | 0.0003 | 0.0037 | 0.0167 |
| | | LOC100128430 | | | | |
| 100128765 | LOC100128765 | similar to hCG2041516 | 13q12.11 | 0.0030 | 0.0161 | 0.0586 |
| 100129122 | LOC100129122 | hypothetical protein | 13q32.3 | 0.0007 | 0.0008 | 0.1120 |
| | | LOC100129122 | | | | |
| 100129174 | RP11-90M2.3 | novel protein similar to polymerase | 13q14.2 | 0.0024 | 0.0009 | 0.2868 |
| | | (RNA) II (DNA directed) | | | | |
| | | polypeptide K, 7.0 kDa POLR2K | | | | |
| 100129303 | LOC100129303 | hypothetical protein | 13q14.3 | 0.0004 | 0.0001 | 0.2743 |
| | | LOC100129303 | | | | |
| 100129390 | LOC100129390 | hypothetical protein | 13q34 | 0.0047 | 0.0020 | 0.3244 |
| | | LOC100129390 | | | | |
| 100129538 | LOC100129538 | hypothetical protein | 13q32.1 | 0.0048 | 0.0012 | 0.4102 |
| | | LOC100129538 | | | | |
| 100129597 | LOC100129597 | hypothetical protein | 13q14.2 | 0.0017 | 0.0006 | 0.2805 |
| | | LOC100129597 | | | | |
| 100129836 | LOC100129836 | hypothetical protein | 13q34 | 0.0023 | 0.0030 | 0.1458 |
| | | LOC100129836 | | | | |
| 100130463 | LOC100130463 | hypothetical protein | 13q34 | 0.0002 | 0.0036 | 0.0120 |
| | | LOC100130463 | | | | |
| 100130563 | LOC100130563 | similar to CNOT4 protein | 13q12.11 | 0.0016 | 0.0014 | 0.1662 |
| 100130779 | LOC100130779 | hypothetical protein | 13q14.11 | 0.0005 | 0.0005 | 0.1241 |
| | | LOC100130779 | | | | |
| 100130979 | LOC100130979 | hypothetical protein | 13q12.11 | 0.0017 | 0.0014 | 0.1803 |
| | | LOC100130979 | | | | |
| 100131110 | LOC100131110 | hypothetical protein | 13q32.3 | 0.0006 | 0.0005 | 0.1281 |
| | | LOC100131110 | | | | |
| 100131435 | LOC100131435 | hypothetical protein | 13q34 | 0.0022 | 0.0015 | 0.2119 |
| | | LOC100131435 | | | | |
| 100131766 | OK/SW-CL.58 | OK/SW-CL.58 | 13q12.3 | 0.0048 | 0.0067 | 0.1706 |
| 100131993 | LOC100131993 | similar to hCG2020760 | 13q14.2 | 0.0017 | 0.0006 | 0.2805 |
| 100132099 | UNQ1829 | FRSS1829 | 13q32.3 | 0.0007 | 0.0008 | 0.1120 |
| 100132761 | LOC100132761 | hypothetical protein | 13q14.3 | 0.0008 | 0.0001 | 0.4005 |
| | | LOC100132761 | | | | |
| 100133284 | LOC100133284 | similar to hCG1791648 | 13q12.13 | 0.0041 | 0.0048 | 0.1859 |

### Example 10

### Expanded Methods for Correlating IRS2 Copy Number, mRNA and Protein Expression Level and BMS-754807 Sensitivity

The following experiments relate to and expand upon the experiments described in Example 4. Materials and methods for these experiments are as described in Example 7 herein.

Since IRS2 DNA copy number was correlated with sensitive to BMS-754807 (FIG. 6A, top), the present inventors then tested whether the IRS2 RNA and protein levels were correlated with IRS2 DNA copy number, and with in vitro sensitivity to BMS-754807. First, the expression levels of RNA and protein in the 60 cell lines were assessed by qRT-PCR and MSD, respectively (Table 3). Pearson correlation analysis showed a good correlation between IRS2 RNA and protein expression levels with a correlation coefficient (r) of 0.448 (p=0.0003). In addition, IRS2 DNA copy number was correlated with both RNA (r=0.3; p=0.02) and protein levels (r=0.39; p=0.0018). Furthermore, compared to the resistant cell lines, significant higher levels of IRS2 RNA (FIG. 6A, middle), as well as a trend of higher protein levels was observed in the sensitive cell lines (FIG. 6A, bottom). These expanded results are consistent with the preliminary results observed in Example 4 herein.

### Example 11

### Expanded Methods for Analyzing KRAS Mutant Cell Lines with IRS2 Amplification and Sensitivity to BMS-754807

The following experiments relate to and expand upon the experiments described in Example 6. Materials and methods for these experiments are as described in Example 7 herein.

The results showed herein that cell lines with either KRAS^{G13D} or BRAF^{V600E} mutations are not sensitive to BMS-754807; however, a subset of KRAS mutations at other positions or in KRAS/BRAF-WT subpopulations were likely to respond to the drug (FIG. 6B). As IRS2 amplification is enriched in the sensitive cell lines (FIG. 5C), the present inventors next explored IRS2 amplification in relation to KRAS mutational status and found that IRS2 amplification was more significantly correlated with the drug sensitivity in KRAS mutated CRC lines, 5 of the 6 amplified lines sensitive to BMS-754807 (FIG. 6C, p=0.01, Fisher exact test). Interestingly, KRAS^{G13D} mutations were all found in the resistant lines and none had IRS2 amplification (FIG. 6C). Whereas there was no apparent correlation between IRS2 amplification status and drug sensitivity in KRAS-WT. Among 4 lines with IRS2 amplification, two with BRAF-WT were sensitive and two with BRAF mutation were resistant to BMS-754807 (FIG. 6D).

These expanded results are consistent with the preliminary results observed in Example 6 herein.

### Example 12

### Expanded Methods for Analyzing Differential Expression Patterns of IGF-1R, IR-A and IGFBP6, and Sensitivity to BMS-754807 in Subpopulations Defined by KRAS and BRAF Status

The following experiments relate to and expand upon the experiments described in Examples 5 and 6. Materials and methods for these experiments are as described in Example 7 herein.

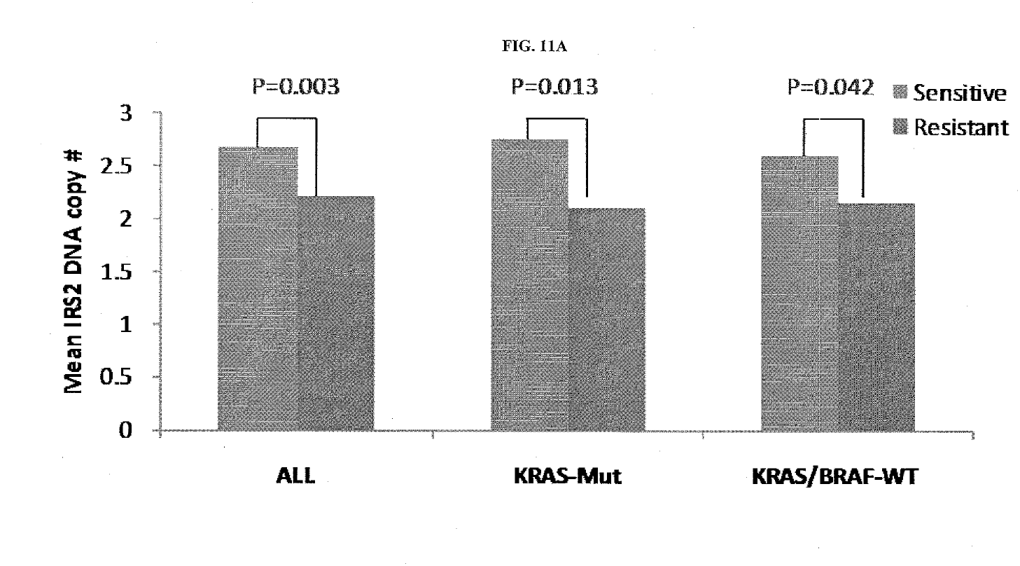

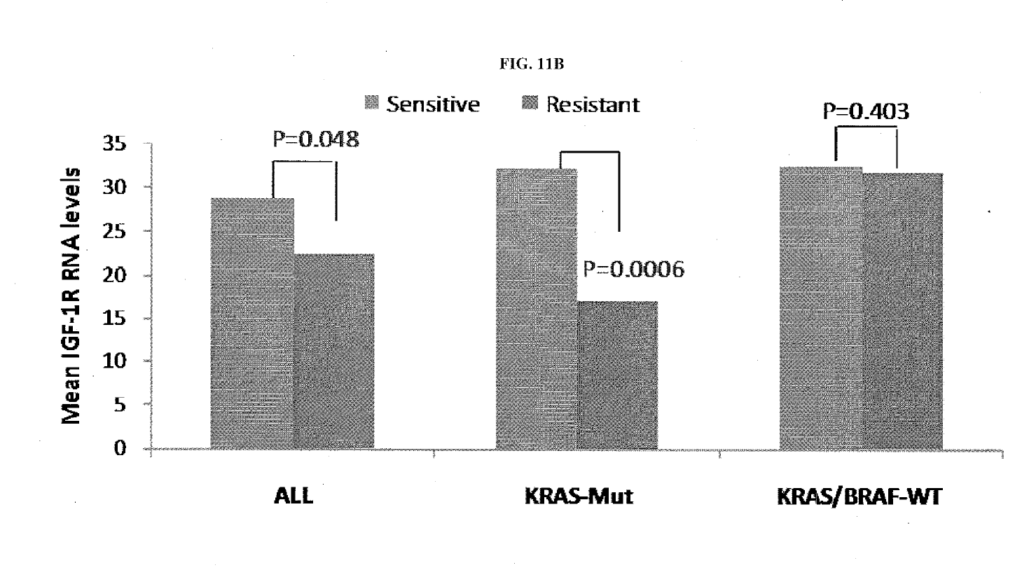

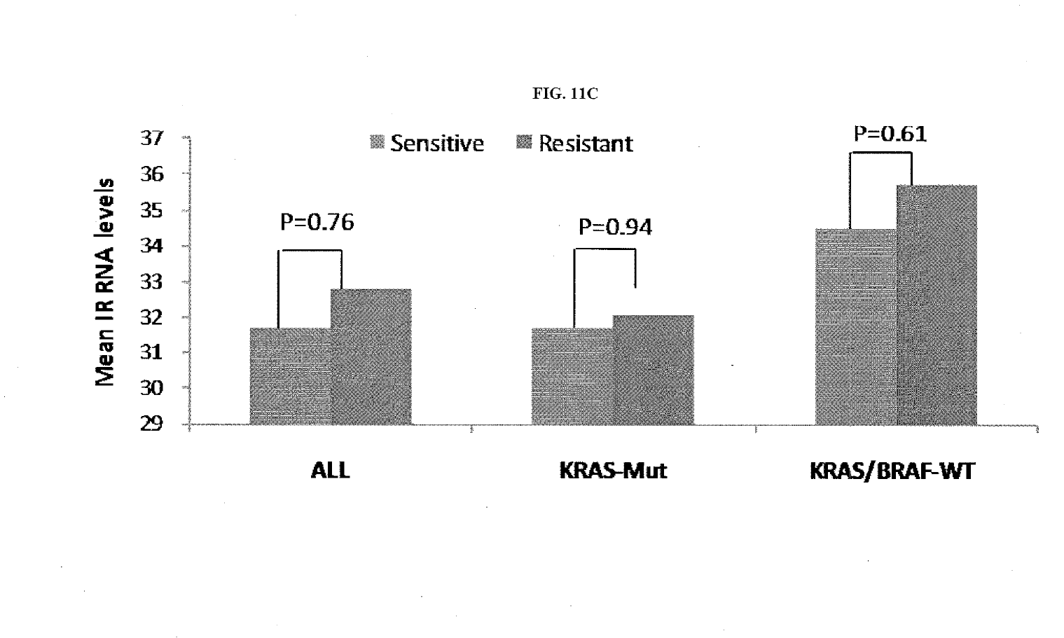

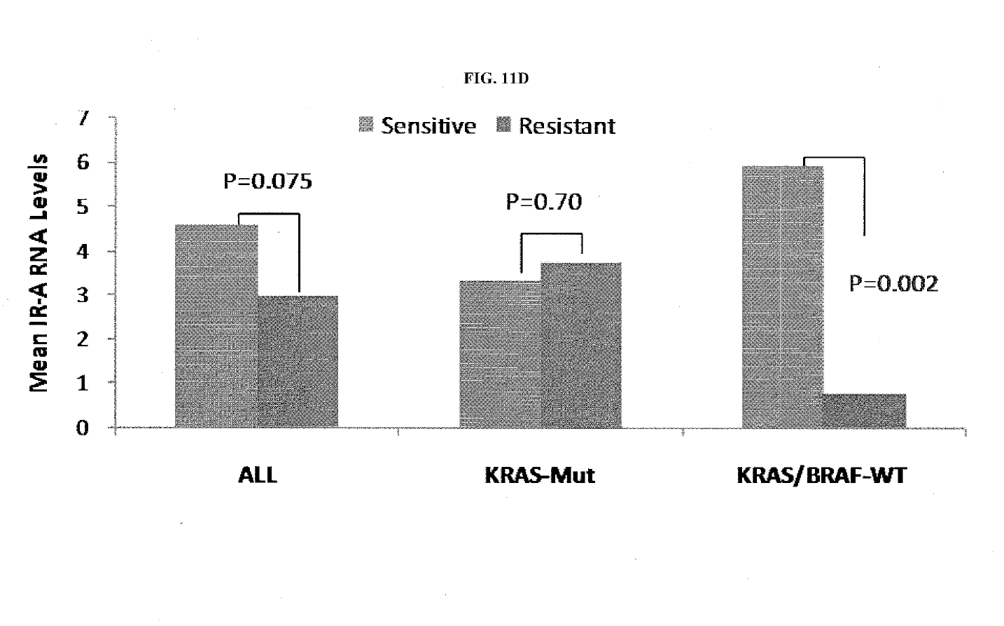

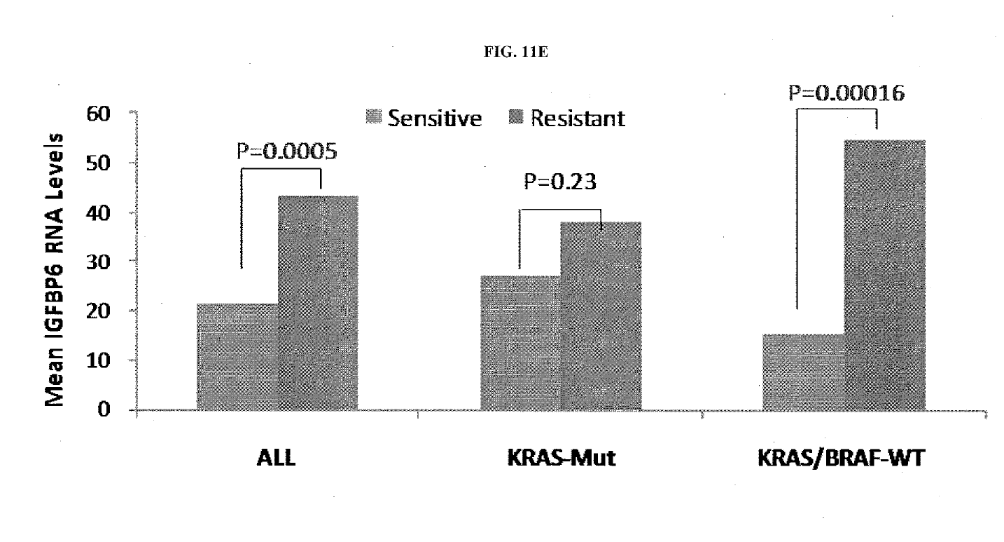

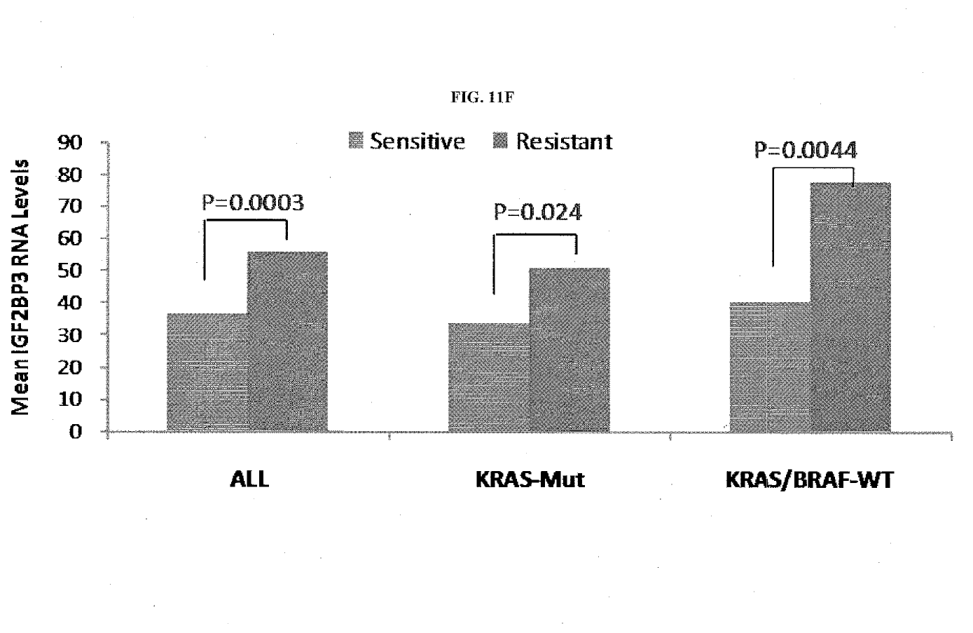

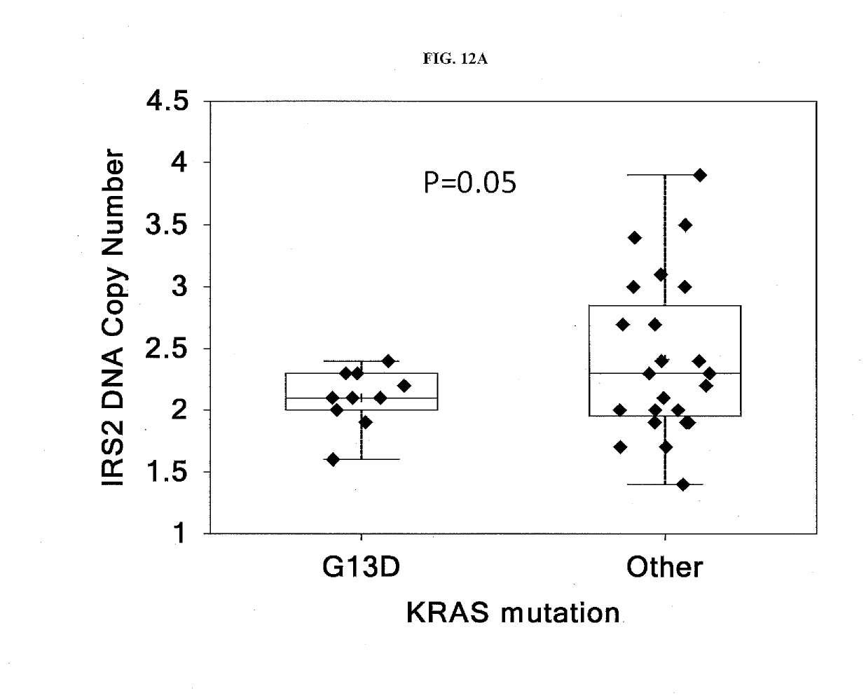

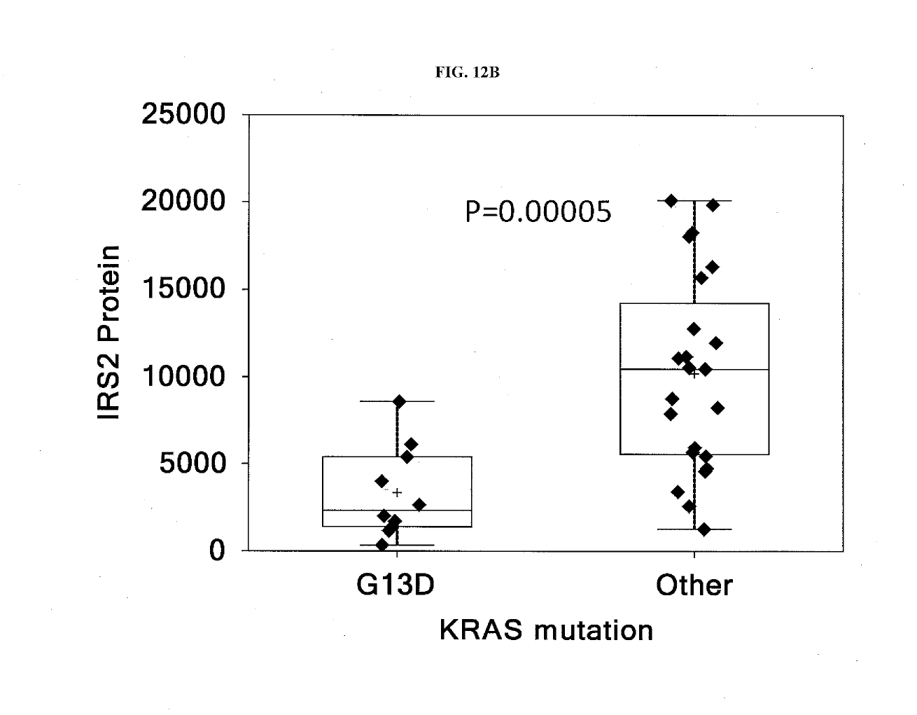

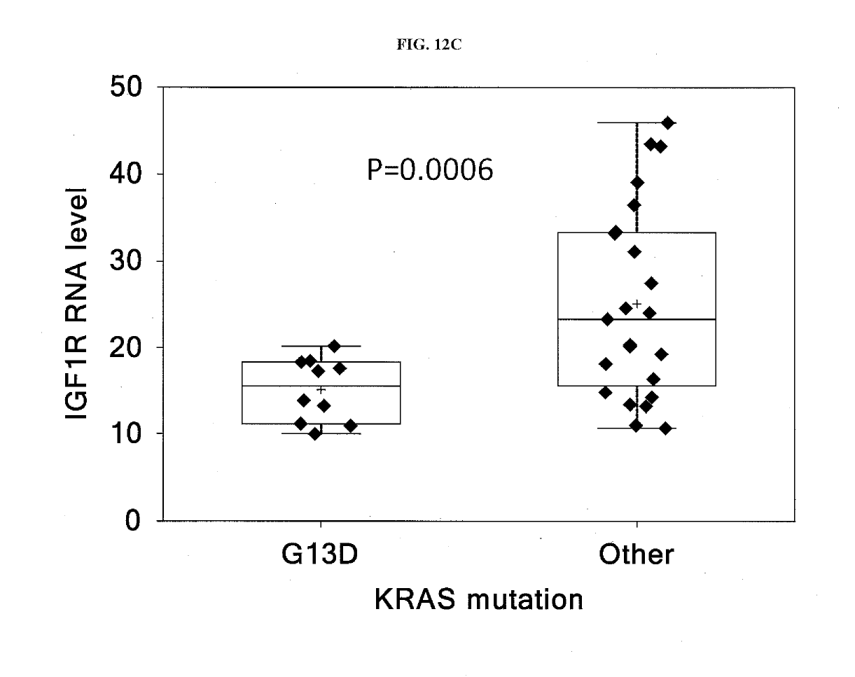

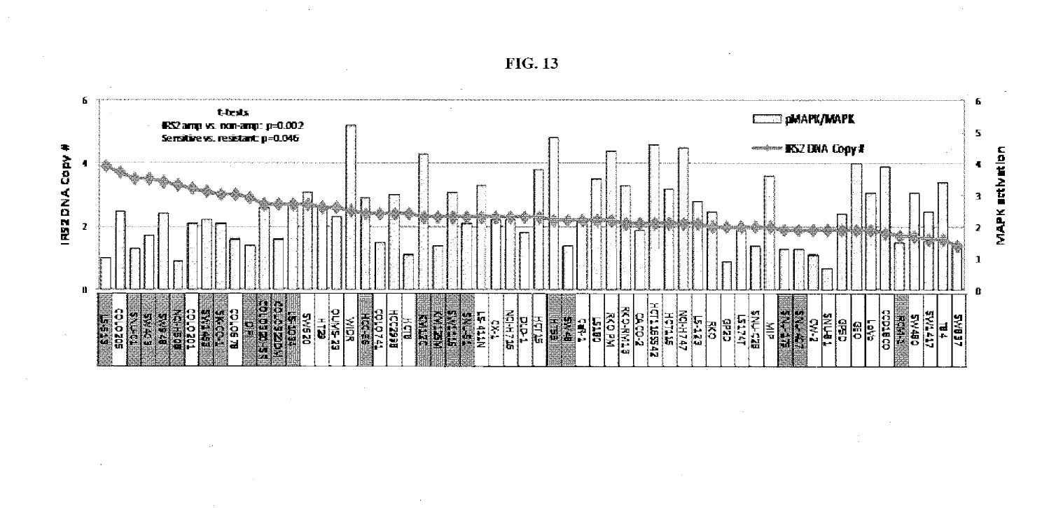

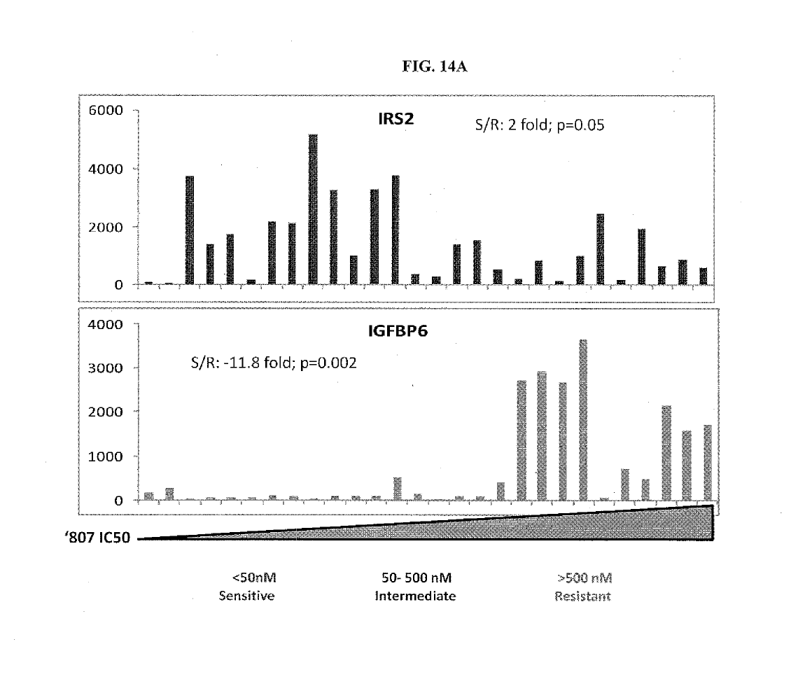

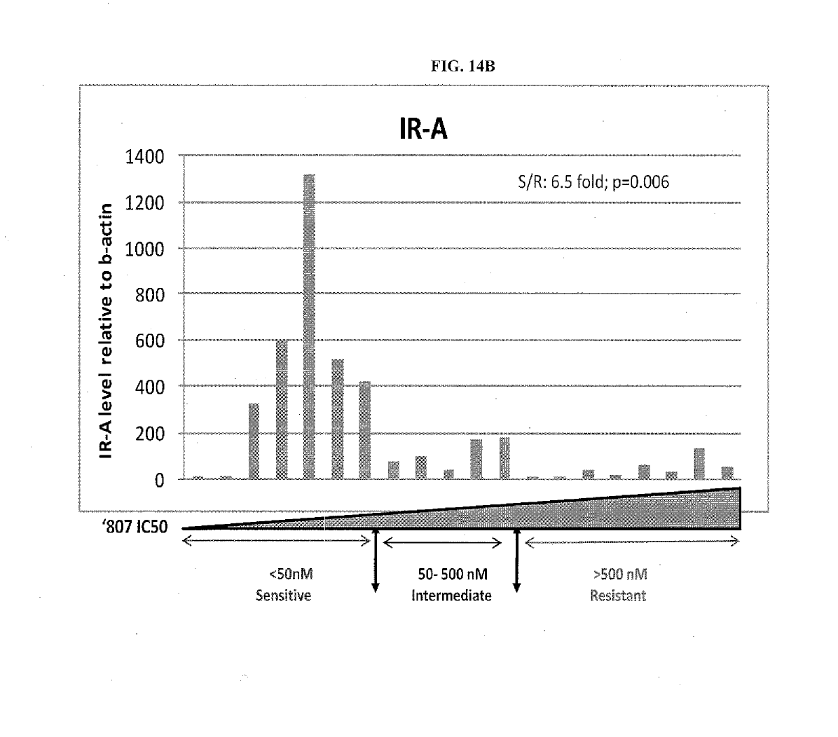

KRAS and BRAF mutational status divides this panel of CRC cell lines into 3 subpopulations: KRAS mutant, BRAF mutant and KRAS/BRAF-WT. Cell lines with BRAF mutation were not sensitive to BMS-754807 (FIG. 6B). The present inventors then evaluated the IGF pathway components by comparing IRS2 CNV and RNA expression levels of receptors, ligands and IGFBPs between sensitive and resistant cell lines in the other two subpopulations, KRAS mutants and KRAS/BRAF-WT. IRS2 DNA copy number was significantly higher in the sensitive cell lines compared to the resistant ones in the whole population (p=0.003), or in KRAS mutants (p=0.013) and KRAS/BRAF-WT (p=0.042) subpopulations (FIG. 11A). For IGF-1R RNA expression, when compared to resistant lines, significantly higher levels were seen in sensitive lines in KRAS mutated subpopulation (p=0.0006, FIG. 6E), but no significant difference was observed in KRAS/BRAF-WT subpopulation (p=0.40; FIG. 11B). However, no significant difference in the levels of total IR (FIG. 11C) or IR-B isoform (data not shown) was apparent. Significantly higher RNA levels of IR-A isoform were observed in the sensitive lines compared to resistant lines (7.6 fold, p=0.002) in KRAS/BRAF-WT only (FIG. 6F), not in KRAS mutants or in the whole population (FIG. 11D). No significant differences in the levels of IGF1 or IGF2 ligands were seen when comparing the sensitive and resistant lines in either subpopulation (data not shown). Interestingly, RNA expression levels of IGFBP6 were significantly lower in sensitive lines compared to resistant lines in KRAS/BRAF-WT subpopulation (p=0.00016, FIG. 6G), in whole population (p=0.0005) but not in KRAS mutated subpopulation (FIG. 11E); none of the other IGFBP levels were significantly associated with sensitivity to BMS-754807 (data not shown). In addition, the RNA levels of IGF2 binding protein 3 (IGF2BP3), was also lower in sensitive lines (FIG. 11F). Interestingly, when compared to cell lines with other KRAS mutations, KRAS^{G13D} cell lines had no IRS2 amplification (FIG. 6C) and significantly lower levels of IRS2 protein and IGF-1R RNA expression (FIG. 12).

These expanded results are consistent with the preliminary results observed in Examples 5 and 6 herein.

### Example 13

### Expanded Methods for Analyzing Cell Lines with IRS2 Amplification to Assess Whether they are More Responsive to Stimulation by IGF-1R Ligands and BMS-754807 Inhibition

The following experiments relate to and expand upon the experiments described in Example 6. Materials and methods for these experiments are as described in Example 7 herein.

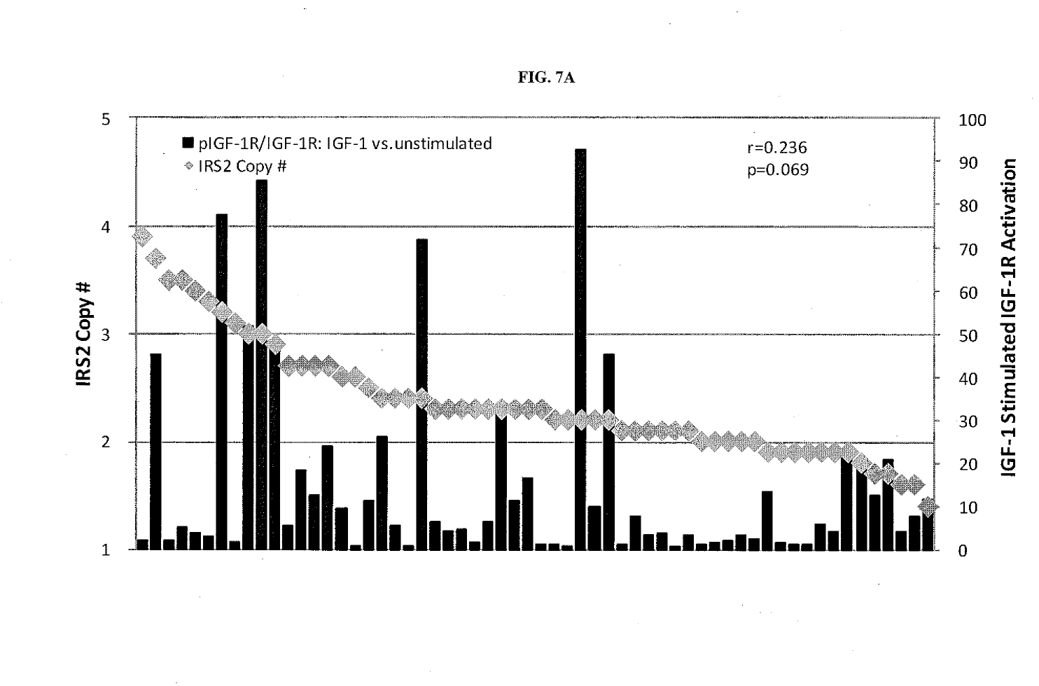

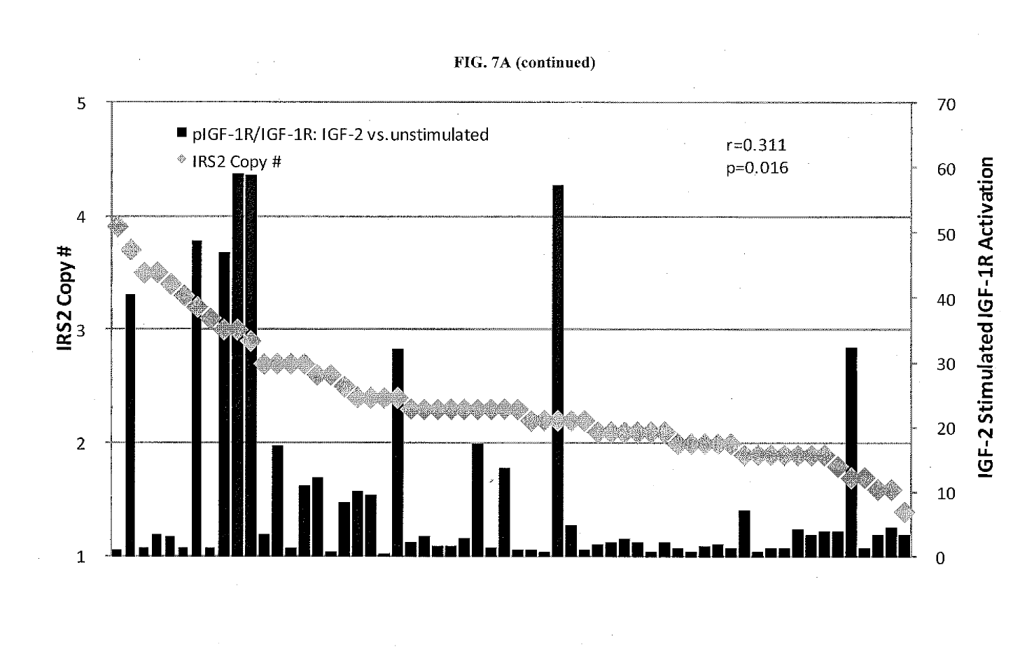

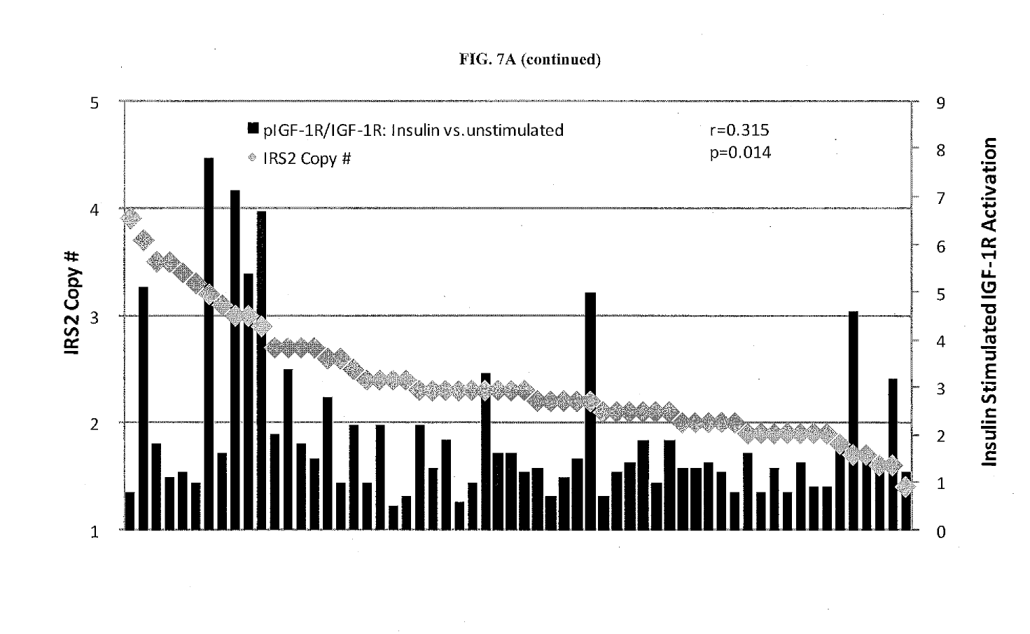

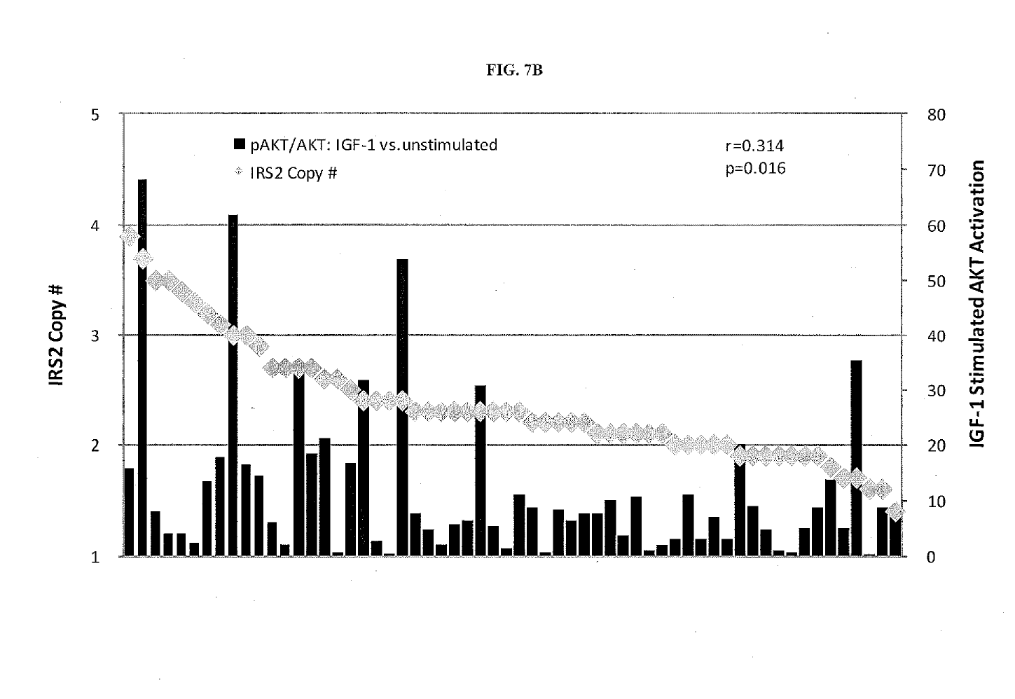

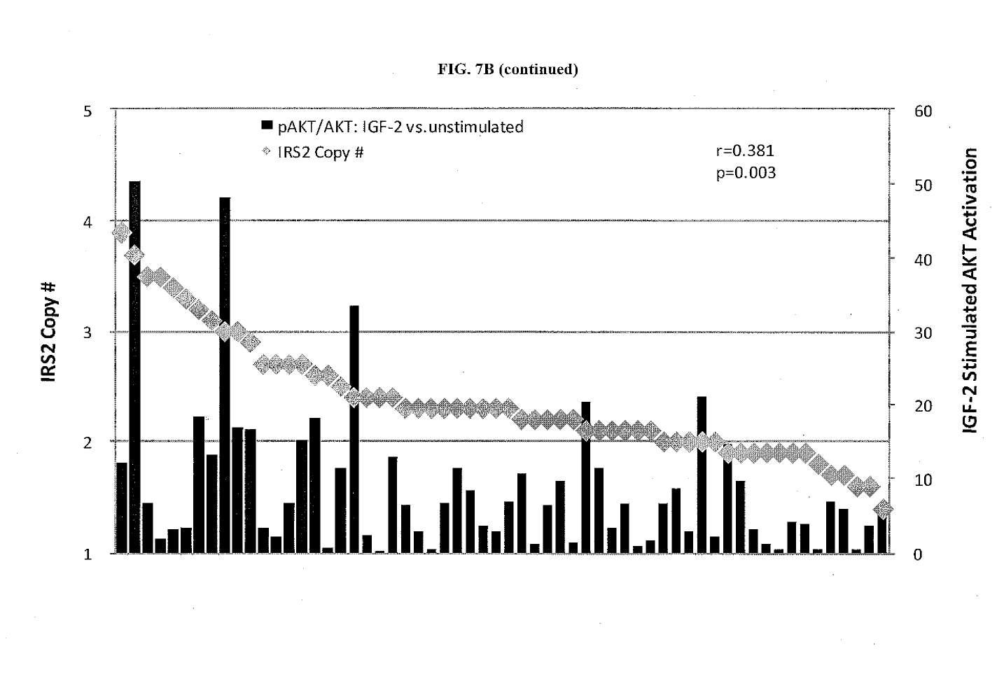

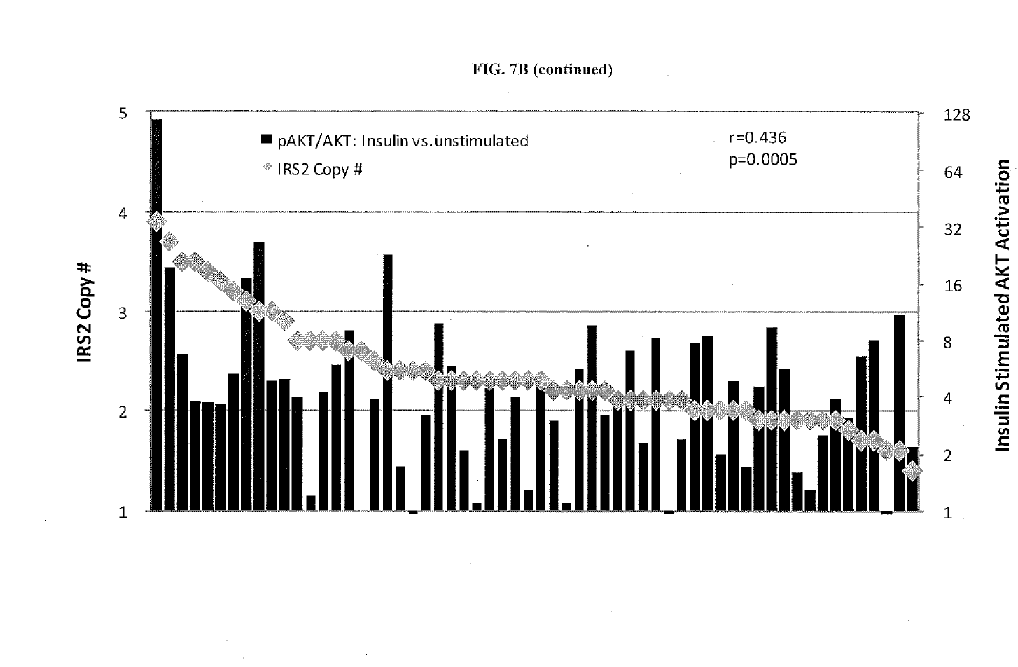

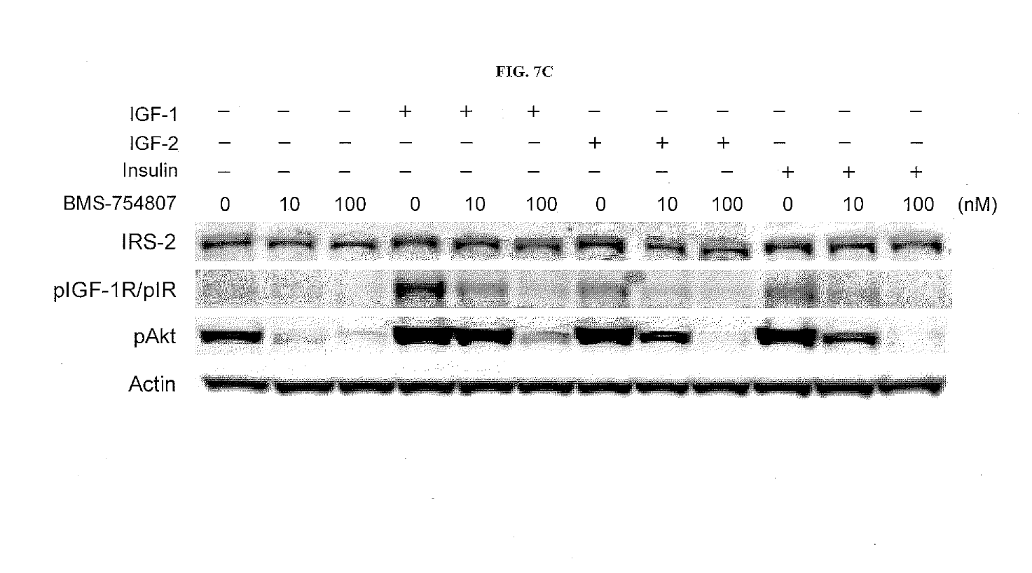

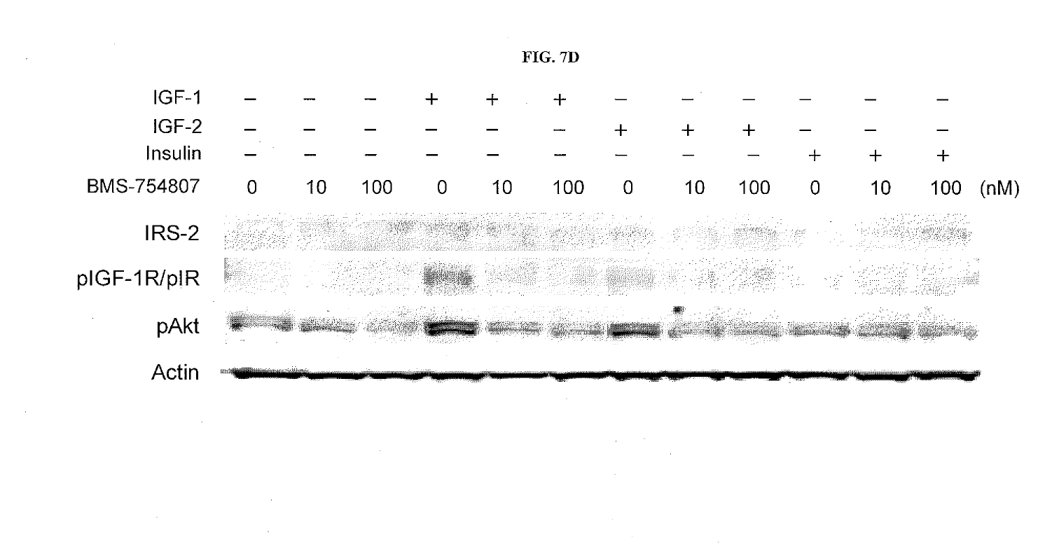

Next, the present inventors performed cell signaling studies to determine differences in IGF signaling pathways at baseline and/or in response to ligand stimulation in relation to BMS-754807 sensitivity and to IRS2 copy numbers. All 60 CRC cell lines were either stimulated with IGF-1, IGF-2 or insulin, or unstimulated. The levels of phospho- and total IGF-1R, IR, IRS1, IRS2, AKT and MAPK were evaluated by both Western blot and MSD analyses. The results showed that IRS2 copy number positively correlated with the levels of ligand-stimulated activation of IGF-1R (FIG. 7A) and AKT (FIG. 7B), which are determined as the ratio of the pIGF-1R/IGF-1R or pAKT/AKT value in specific ligand-stimulated cells vs. the ratio in the non-stimulated cells, suggesting that IGF-IR signaling pathways were more actively coupled to AKT signaling in response to ligand activation in IRS2 amplified cell lines compared to non-amplified lines. In addition, IRS2 amplified cell lines had significantly lower basal levels of MAPK activation than non-amplified cell lines (p=0.002) and similar results were observed in the sensitive cell lines compared to resistant lines (FIG. 13), suggesting that non-amplified IRS2 or resistant cell lines had higher activation of MAPK pathway.

To further explore the mechanisms of differential response to BMS-754807 between KRAS mutated cell lines with IRS2 amplification and those with normal copy numbers, cells were treated with 10 or 100 nM of BMS-754807 for 1 hr, then stimulated with IGF-1, IGF-2, or insulin for 10 min. Cell lysates were subsequently subjected to Western blot analysis and evaluated for pIGF-1R/pIR and pAKT. SK-CO-1 cells with IRS2 amplification had higher expression levels of IRS2 protein; pIGF-1R/pIR and pAKT levels increased in response to individual ligand stimulation, and were inhibited by BMS-754807 treatment in a dose-dependent manner (FIG. 7C). Similar results were observed for LS513 and SW-403 cell lines, which are also KRAS mutant and IRS2 amplified (data not shown). On the contrary, DLD-1 with normal IRS2 copy number and low to undetectable levels of IRS2 protein expression, showed a limited response to IGF-2 or insulin stimulation for pIGF-1R/pIR and pAKT activation, and was not significantly inhibited by BMS-754807 (FIG. 7D).

These expanded results are consistent with the preliminary results observed in Example 6 herein.

### Example 14

### Expanded Methods for Analyzing Modulation of IRS2 Levels with Sensitivity to BMS-754807

The following experiments relate to and expand upon the experiments described in Example 5. Materials and methods for these experiments are as described in Example 7 herein.

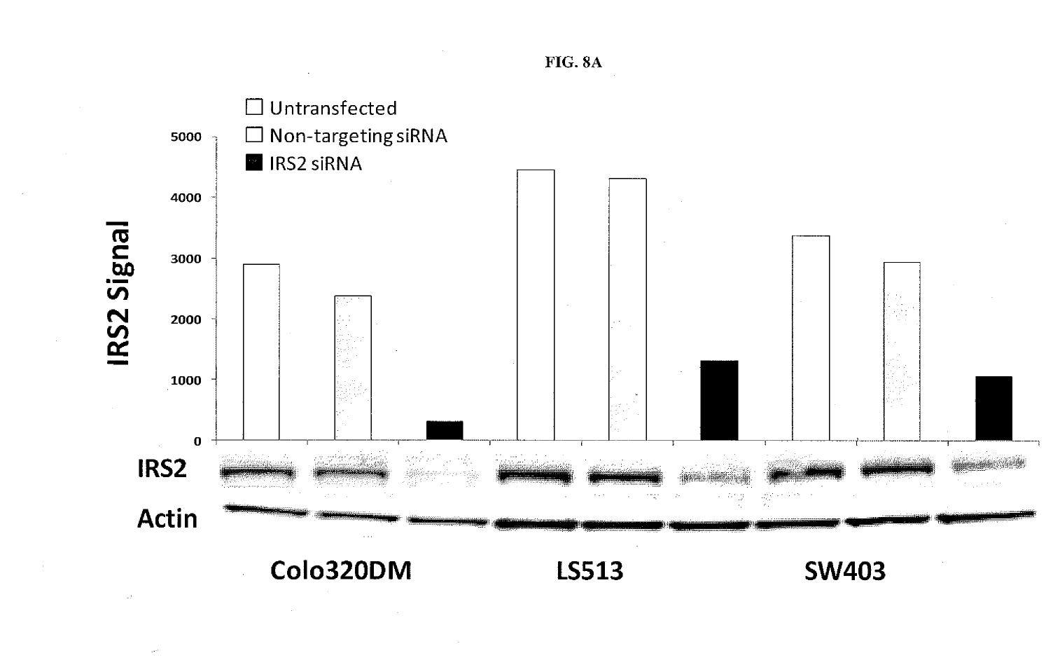

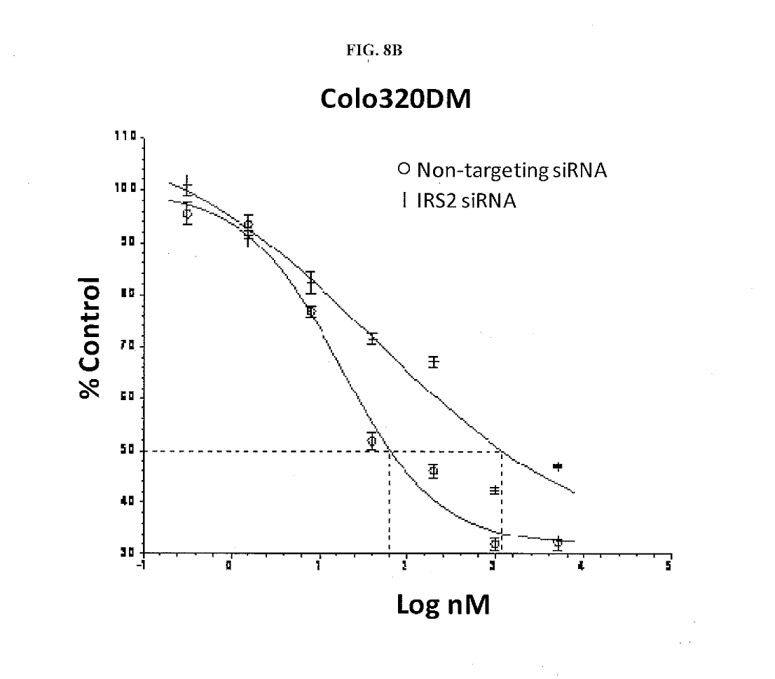

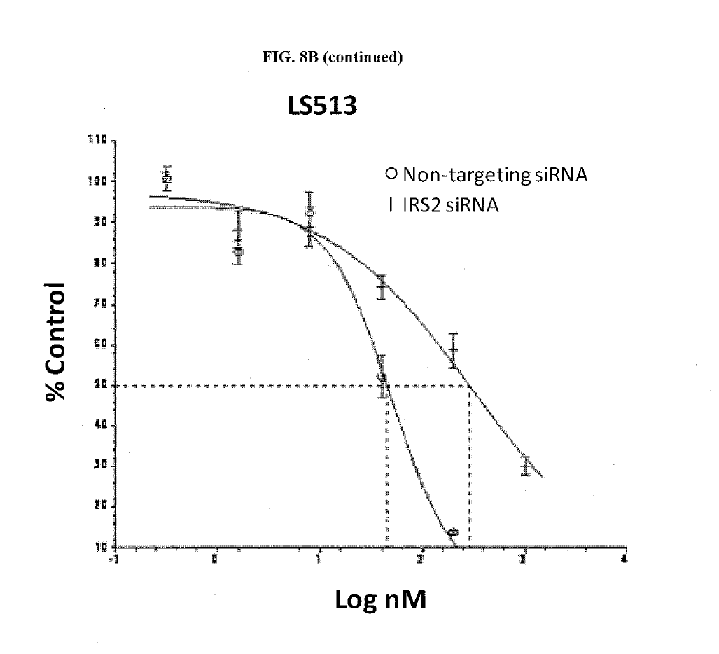

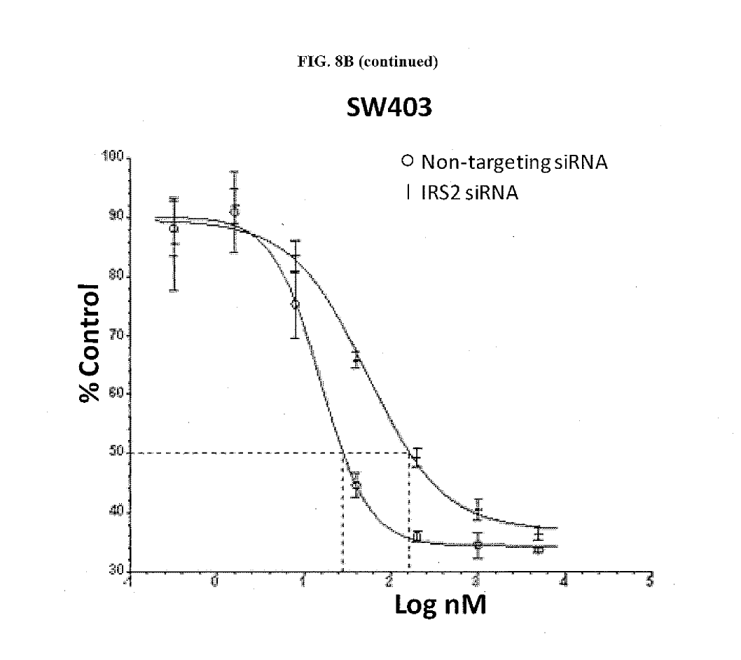

To investigate the role of IRS2 in relation to the sensitivity to BMS-754807, the present inventors utilized siRNA studies to knockdown the IRS2 expression level in 3 cell lines that were sensitive to BMS-754807; the cells were either KRAS-WT (COLO320DM) or mutant (LS-513, SW403). After transfection, the cells were exposed to BMS-754807 at different concentrations for 72 hours and cell proliferation was used to assess their sensitivity profiles. As shown in FIG. 4A, IRS2 siRNA significantly decreased the expression level of IRS2 protein as verified by Western Blot and MSD analyses when compared to the cells transfected with a non-targeting control siRNA and in un-transfected cells. Knockdown of IRS2 in all 3 cell lines resulted in a shift in the proliferation curves with increased IC₅₀ values compared with the non-targeted control siRNA, indicating reduction of sensitivity to BMS-754807 (FIG. 8B). These results supported the observation that cell lines with higher expression levels of IRS2 were more sensitive to BMS-754807, and down-regulation of IRS2 levels decreased the response to the drug. In addition, the present inventors also conducted IRS2 overexpression studies in the DLD-1 cell line, which had lower levels of IRS2 expression and was resistant to BMS-754807. Transfection with IRS2 plasmid DNA to increase the IRS2 expression level resulted in an increase in sensitivity to the drug by shifting to a lower IC₅₀ (data not shown). These results provided evidence that IRS2 has a functional role in mediating sensitivity to IGF-1R/IR inhibitor BMS-754807.

These expanded results are consistent with the preliminary results observed in Example 5 herein.

### Example 15

### Expanded Methods for Analyzing IRS2 Amplification and Establishing Increased Prevalent in CRC than in Other Tumor Types

The following experiments relate to and expand upon the experiments described in Example 6. Materials and methods for these experiments are as described in Example 7 herein.

As IRS2 DNA amplification status is associated with sensitivity of BMS-754807 and modulation of IRS2 expression level altered the response to the drug, IRS2 amplification could be used as a potential predictive biomarker for patient selection. To estimate the size of the targeted population, the present inventors next assessed the prevalence of IRS2 amplification in cancer and especially in CRC. By data mining of publicly available sources on tumor annotations (Table 6), the percentage of IRS2 amplification in CRC, as measured by SNP array, ranged from 8-26% in a total 648 samples from 4 datasets, which is higher than in any other tumor types (0-2.9%). For examples, the prevalence of IRS2 amplification is 2.9% (20/699), 2.6% (16/608), 1.8% (16/911) and 1.9% (3/154) in breast, ovary, lung and liver cancers, respectively. IRS2 amplification was not seen in prostate (0/165), renal cancers (0/593) and ALL (0/378).

To further stratify the prevalence of IRS2 amplification by KRAS mutational status, the present inventors subsequently analyzed 94 formalin fixed paraffin embedded (FFPE) CRC specimens either from primary or metastatic tumors for IRS2 copy number by qPCR CNV and KRAS mutational status by Sanger sequencing. The results from this limited number of samples indicated that the prevalence of IRS2 amplification was ˜35%, with no significant differences observed between primary (35.7%) and metastatic CRC tumors (33%) or between KRAS-WT (33.8%) and mutated (38.5%) populations (Table 7).

These expanded results are consistent with the preliminary results observed in Example 6 herein.

TABLE 6
The prevalence of IRS2 amplification in different
tumor types by SNP analysis via data mining.

| | | | # with | % of | |
|---|---|---|---|---|---|
| | | | IRS2 > | IRS2 > | |
| Cancer Type | Data Source | N | 3 cp | 3 cp | Assay type |
| Colon cancer | GEO, | 48 | 4 | 8.3% | Mapping250K |
| | GSE16125 | | | | |
| Colon | TCGA | 348 | 69 | 19.8% | SNP6 chip |
| adenocar- | | | | | |
| cinoma | | | | | |
| Rectum | TCGA | 124 | 32 | 25.8% | SNP6 chip |
| adenocar- | | | | | |
| cinoma | | | | | |
| Head and | TCGA | 91 | 0 | 0.0% | SNP6 chip |
| Neck | | | | | |
| Squamous | | | | | |
| cell | | | | | |
| carcinoma | | | | | |
| (HNSC) | | | | | |
| Breast | TCGA | 506 | 18 | 3.6% | SNP6 chip |
| Lung_{—} | TCGA | 167 | 3 | 1.8% | SNP6 chip |
| squamous | | | | | |
| Lung_{—} | TCGA | 98 | 3 | 3.1% | SNP6 chip |
| adenocar- | | | | | |
| cinoma | | | | | |
| Stomach | TCGA | 109 | 2 | 1.8% | SNP6 chip |
| Ovarian | TCGA | 513 | 16 | 3.1% | SNP6 chip |
| serous | | | | | |
| cystadeno- | | | | | |
| carcinoma | | | | | |
| (OV) | | | | | |
| Renal clear | TCGA | 494 | 0 | 0.0% | SNP6 chip |
| cell | | | | | |
| carcinoma | | | | | |
| Liver | TCGA | 44 | 2 | 4.5% | SNP6 chip |
| hepato- | | | | | |
| cellular | | | | | |
| carcinoma | | | | | |
| (LIHC) | | | | | |
| Prostate | TCGA | 82 | 0 | 0.0% | SNP6 chip |
| adenocar- | | | | | |
| cinoma | | | | | |
| (PRAD) | | | | | |
| Acute | Tumorscape | 378 | 0 | 0.0% | Mapping250K + |
| lympho- | | | | | others |
| blastic | | | | | |
| leukemia | | | | | |
| Breast | Tumorscape | 193 | 2 | 1.0% | Mapping250K + |
| | | | | | others |
| Colorectal | Tumorscape | 128 | 14 | 10.9% | Mapping250K + |
| | | | | | others |
| Hepato- | Tumorscape | 110 | 1 | 0.9% | Mapping250K + |
| cellular | | | | | others |
| Lung NSC | Tumorscape | 629 | 9 | 1.4% | Mapping250K + |
| | | | | | others |
| Lung SC | Tumorscape | 17 | 1 | 5.9% | Mapping250K + |
| | | | | | others |
| Medullo- | Tumorscape | 119 | 3 | 2.5% | Mapping250K + |
| blastoma | | | | | others |
| Ovarian | Tumorscape | 95 | 0 | 0.0% | Mapping250K + |
| | | | | | others |
| Prostate | Tumorscape | 83 | 0 | 0.0% | Mapping250K + |
| | | | | | others |
| Renal | Tumorscape | 99 | 0 | 0.0% | Mapping250K + |
| | | | | | others |

TABLE 7
The prevalence of IRS2 amplification stratified
by KRAS status in CRC tumor samples. The ISR2
CNV was measured by qPCR CNV assay.

| | Total | | KRAS-WT | | KRAS-Mutation | |
|---|---|---|---|---|---|---|
| | | Total N with | | Total N with | | Total N with |
| | | IRS2 >= 3 | | IRS2 >= 3 | | IRS2 >= 3 |
| Sample | N | (%) | N | (%) | N | (%) |
| Primary | 70 | 25 (35.7%) | 54 | 19 (35.2%) | 16 | 6 (37.5%) |
| tumors | | | | | | |
| Metastatis | 24 | 8 (33%) | 14 | 4 (28.6%) | 10 | 4 (40%) |
| tumors | | | | | | |
| Total | 94 | 33 (35.1%) | 68 | 23 (33.8%) | 26 | 10 (38.5%) |

### CONCLUSION

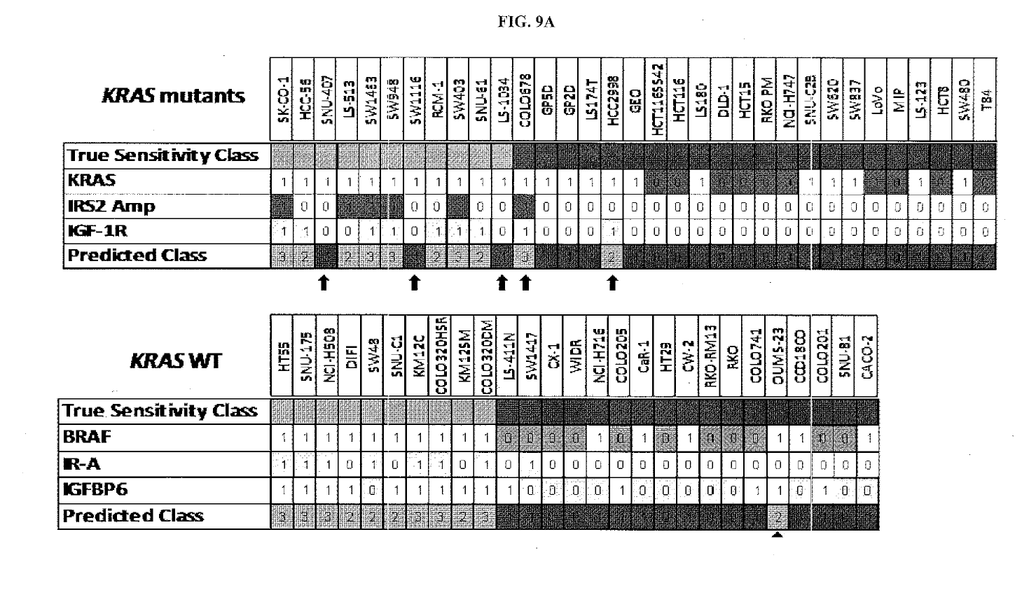

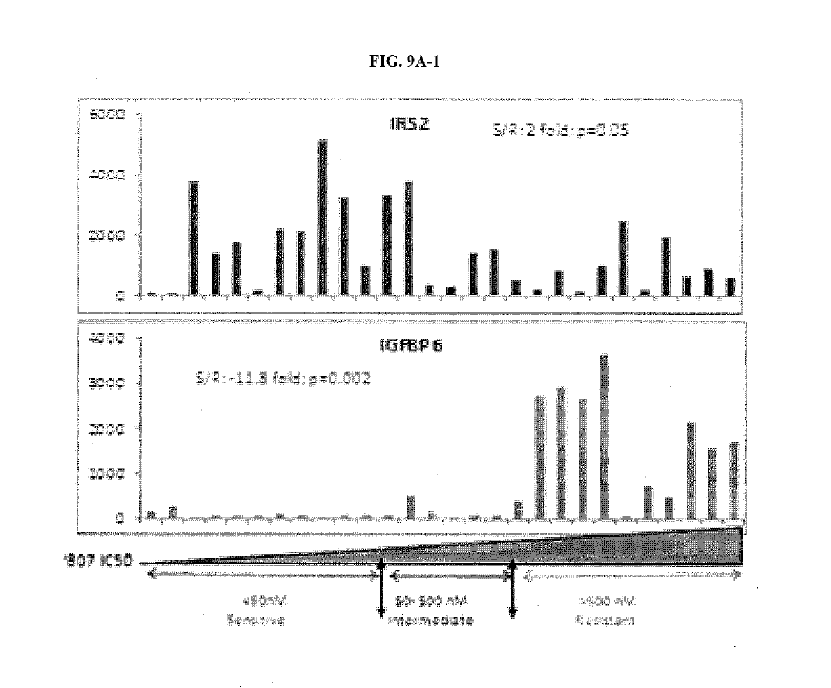

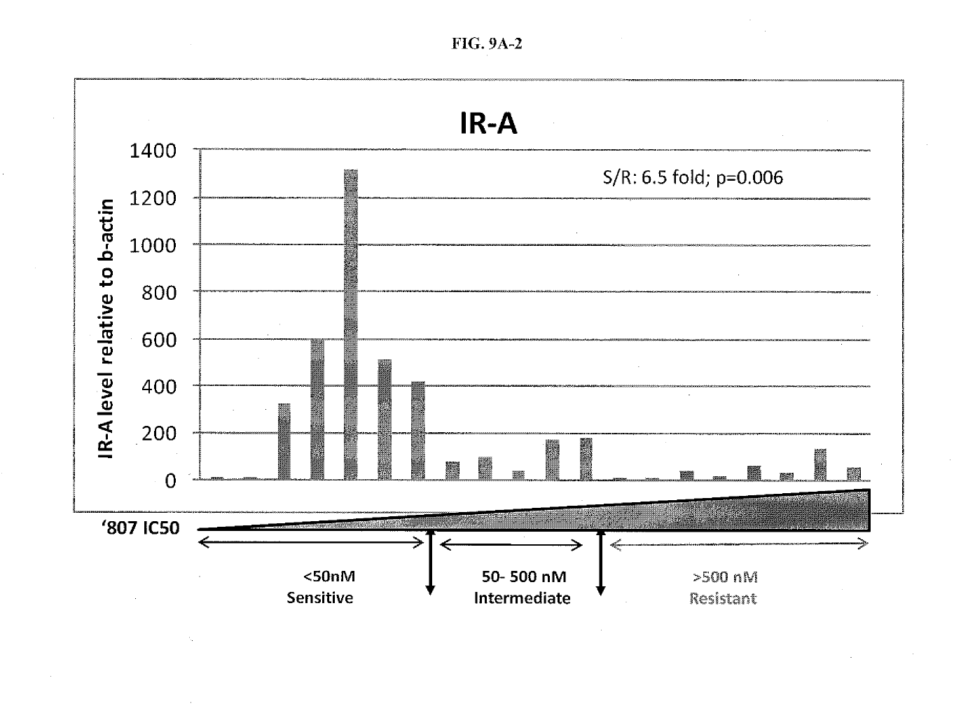

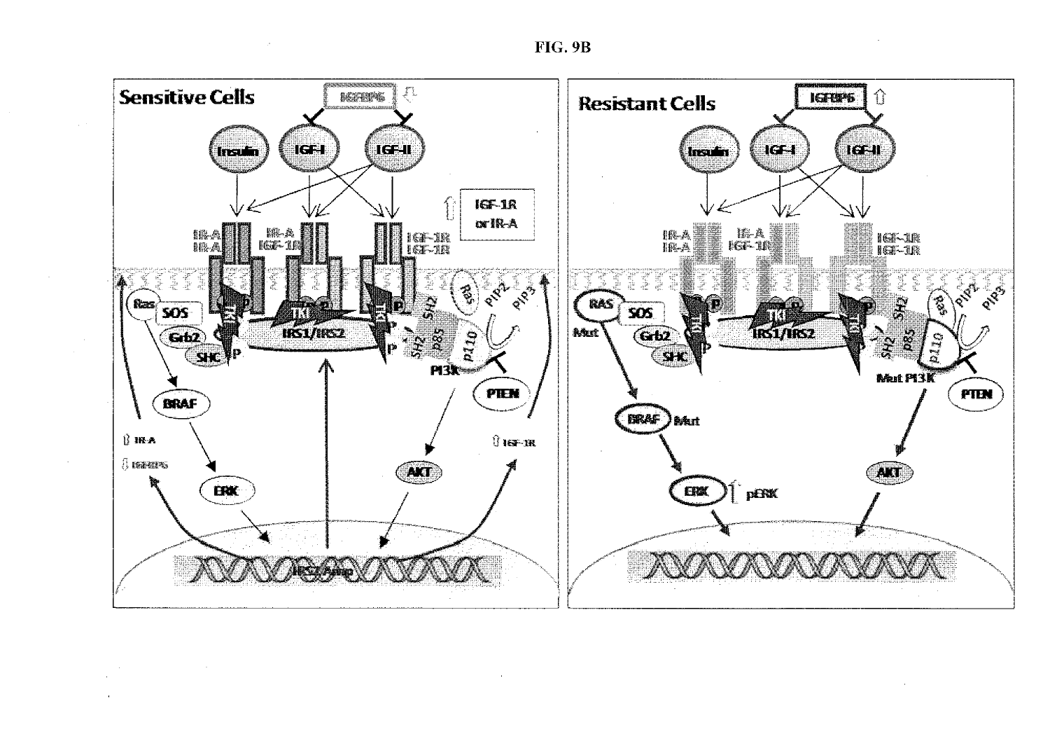

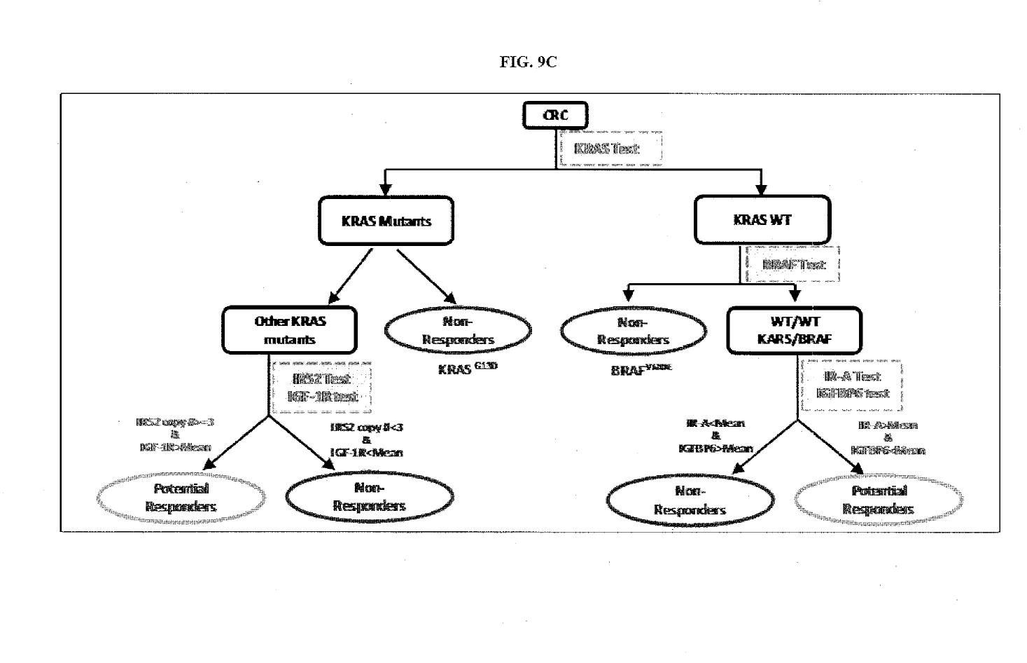

The NCI-60 cell line panel and associated drug screens pioneered the approach of using cancer cell lines to link drug sensitivity with genotype data (30, 31). Cancer cell lines have subsequently been used to identify rare drug-sensitizing genotypes, including mutant EGFR, BRAF and the EML4-ALK translocations, which are highly predictive of clinical responses (32-34). More recently, two published reports took the pharmacology of cultured cancer cells to the next level by including an extensive compilation of gene expression, chromosome copy number, and sequencing data on a panel of several hundred diverse cancer cell lines along with their sensitivity to over a hundred different anticancer agents (35, 36). These studies provided highly useful, large-scale resources for the generation and testing of hypotheses related to the overall goal of personalizing cancer medicine (37). In this study, the present inventors elucidated potential predictive markers of response to the IGF-1R/IR tyrosine kinase inhibitor, BMS-754807, by testing drug sensitivity in a panel of 60 CRC cell lines coupled with systematic genomic analysis. As illustrated in FIG. 9A, the present inventors discovered that: 1) in KRAS mutated cell lines, KRAS^{G13D} is not sensitive to BMS-754807, whereas IRS2 amplification and/or higher IGF-1R RNA expression levels are associated with increased drug sensitivity; 2) cell lines with BRAF^{V600E} mutation are not sensitive to the drug; and 3) in KRAS/BRAF-WT cell lines, the ones having higher IR-A and/or lower IGFBP6 RNA expression levels, are more sensitive to BMS-754807. Utilizing KRAS and BRAF mutational status, IRS2 amplification, IGF-1R, IR-A and IGFBP6 RNA expression level, the present inventors were able to correctly classify the responsiveness to BMS-754807 in 90% (54/60) of CRC cell lines.

CRC is a heterogeneous disease defined by different activating mutations or loss-of-function mutations in KRAS/BRAF/PI3K/PTEN intracellular pathways that impact the efficacy of targeted therapies (38, 39). KRAS has the ability to activate multiple downstream signaling pathways, including PI3K/AKT and MEK/MAPK that have been implicated as independent drivers of tumorigenesis. Our study demonstrated that all cell lines harboring KRAS^{G13D} mutations were resistant to BMS-754807, whereas KRAS mutations at other positions were not significantly correlated with sensitivity to the drug (FIG. 6B). These findings are interesting to note that this observation is opposite for response to EGFR antibody-targeted therapies such as cetuximab. It is generally accepted that the presence of KRAS mutations in metastatic CRC predicts lack of benefit for treatment with cetuximab (40). However, beneficial effects of cetuximab in chemotherapy-refractory metastatic CRC patients with KRAS^{G13D} mutations were seen in a large retrospective pooled exploratory analysis (41). KRAS mutations in codon 12 and 13 have functional and molecular differences in the regulation of apoptosis, cell-cell contact inhibition and predisposition to anchorage-independent growth by the differential regulation of KRAS downstream pathways (42). IGF-1R and EGFR pathways cross-talk and interact to drive tumor growth and survival. Each is activated reciprocally as an escape mechanism when inhibiting one or the other (43). It is unclear why KRAS^{G13D} determines response to IGF-1R/IR and EGFR inhibitors differently, the present inventors found that KRAS^{G13D} cell lines had no IRS2 amplification (FIG. 6C), significantly lower levels of IRS2 protein and IGF-1R RNA expression compared to cell lines with other KRAS mutations (FIG. 12). It is not yet clear, mechanistically, why KRAS^{G13D} mutation co-occurs with the changes in the IGF-1R pathway, but this may be one of reasons why KRAS^{G13D} cell lines are less responsive to BMS-754807 than other KRAS mutants. The correlation between KRAS^{G13D} and response to IGF-1R/IR inhibitors should be validated clinically. The spectrum of KRAS mutations may be critical when assessing the biological behavior of tumors in different clinical settings.

KRAS or BRAF mutations frequently manifest in constitutive activation of the MEK/MAPK signaling pathway. The BRAF protein is located downstream of KRAS and is its principal downstream effector. V600E is an activating mutation of BRAF and results in constitutive activation of the MAPK pathway. In our study, CRC cell lines with BRAF^{V600E} mutations were not sensitive to BMS-754807 (FIG. 6B), and the resistant lines appeared to have higher baseline levels of MAPK activation (FIG. 13), supporting MAPK activation as one of the resistance mechanisms to IGF-1R/IR TKIs (17). Co-targeting MEK and IGF-1R/IR in CRC has been shown to lead to a loss of AKT and ERK activity, marked growth suppression, and robust apoptosis compared with either single-agent EGFR, MEK, or IGF-1R inhibitors or combined EGFR and IGF-IR inhibitors in human KRAS mutant CRC in vitro and in vivo (44), these data supported clinical testing of combining MEK with IGF-1R/IR inhibitors.

Mutations in the PI3KCA gene occur in 12-30% of CRC (45). Most of these mutations are single amino acid substitutions located in hot spots in the helical (exon 9) or kinase domains (exon 20) leading to constitutive activation of the PI3K/AKT signaling pathway (46). The gain-of-function mutation in exon 9 is independent of binding to the p85 regulatory subunit and requires interaction with RAS. In contrast, exon 20 mutations are active in the absence of RAS binding, but are highly dependent on the interaction with p85 (47). PI3K is also an important mediator in the IGF-1R/IR pathway. Our results (FIGS. 5B and 5C) showed that 9 of 10 cell lines with PI3KCA mutation in exon 20 were resistant to BMS-754807, and none of them were IRS2 amplified; cell lines with mutations in exon 9 and with IRS2 amplification were sensitive, whereas the lines without IRS2 amplification were resistant to the drug. It may be important to assess PI3KCA mutations in clinical trials of IGF-1R/IR inhibitors in CRC and determine their association with clinical benefit. PI3K-initiated signaling is inhibited by phosphatase and tensin homologue (PTEN). PTEN activity can be lost through various mechanisms, including mutations in PTEN. Only two of 60 cell lines had PTEN mutation; therefore, the present inventors were not able to assess its association with sensitivity to BMS-754807. There was no significant correlation observed between PTEN DNA copy number and the sensitivity to BMS-754807 (data not shown).

Our results demonstrate that IRS2 amplification and expression is associated with sensitivity to BMS-754807 (FIG. 6A). Utilization of IRS2 as a candidate predictive biomarker is biologically plausible as it is a direct target of IGF-1R/IR and plays a key role in transducing IGF-1R/IR signaling to RAS/ERK and PI3K/AKT pathways, leading to cell proliferation and survival (2, 3). Interestingly, the association between IRS2 amplification and sensitivity to BMS-754807 is more significant in KRAS mutant (FIG. 6C) than in WT cell lines (FIG. 6D). This may be due to the fact that KRAS mutated CRC tumors with IRS2 amplification have IGF-1R/IR pathway activation and are possibly more dependent on IGF-1R/IR pathways for growth. This hypothesis is supported by a recent report showing that IGF-1R has dominant control over PI3K signaling in KRAS mutated CRC. KRAS is an important activator of the ERK pathway, but mutated KRAS does not drive PI3K pathway activity, rather it is driven by IGF-1R activity through interaction of PI3K and IRS1/IRS2 (44). Indeed, KRAS mutated cell lines with higher IRS2 copy number tended to respond better to ligand-stimulated activation of IGF-1R and AKT, and were more responsive to BMS-754807 inhibition (FIG. 7C) compared to cell lines with normal copy number of IRS2 (FIG. 7D).

IGF-1R and IGFBP6 levels have been reported to be associated with sensitivity to IGF-1R/IR inhibitors in several studies (15, 18, 19). The present inventors observed in this study that sensitive cell lines had higher levels of IGF-1R RNA expression, especially in CRC cell lines with KRAS mutations (FIG. 11B) while lower levels of IGFBP6 were seen in sensitive lines (FIG. 11E). IGFBPs are important members of the IGF axis; they regulate the IGF-I pathway and influence IGF signaling by modulating the biological accessibility and activity of the IGFs (15). Cells with lower level of IGFBP6 may have higher IGF-1R pathway activation, therefore more susceptible to IGF-1R inhibition.

Increasing knowledge of the role of IR-A in cancer has important implications for anticancer treatments. Activation of IR signaling or increased expression of the IR-A isoform was observed in cancer cell lines when treated with a selective anti-IGF-1R antibody (13, 48) supporting the notion that activation of the IR-A/IGF2 autocrine loop represents a mechanism of resistance to IGF-1R antibody therapies. Our results demonstrate that KRAS/BRAF-WT cell lines with higher expression of IR-A were more sensitive to BMS-754807 than cells with lower IR-A RNA levels (FIG. 6F), supporting co-targeting IGF-1R and IR with a dual inhibitor such as BMS-754807, which may have enhanced efficacy against biomarker-selected tumors compared with an inhibitor, such as an IGF-1R mAb, that targets only IGF-1R.

Taken together, the present inventors hypothesize (FIG. 9B) that sensitive cells are more dependent on the IGF-1R/IR pathway as the predominant driver for proliferation; this can occur either via IRS2 amplification and/or higher expression of IGF-1R in KRAS mutated cells, or via higher expression of IR-A, and lower expression of IGFBP6 in KRAS/BRAF-WT cells. Activation of IGF-1R/IR pathway results in increased sensitivity to IGF-1R/IR TKI inhibition, leading to decreased downstream PI3K/AKT and RAS/RAF/ERK signaling and consequently decreased in cell proliferation. Whereas resistant cells are less activated in IGF-1R/IR pathways and have dysregulation of ERK and AKT pathways due to KRAS, PIK3CA, or BRAF mutations, making them less dependent on IGF-1R/IR signaling for proliferation; although targeting IGF-1R/IR with a TKI still inhibits IGF-1R/IR activity, it does not sufficiently inhibit the activity of downstream pathways caused by the indicated mutations.

In summary, the present inventors have identified a panel of candidate biomarkers, including KRAS and BRAF mutations, IRS2 amplification, IGF-1R, IR-A and IGFBP6 RNA expression levels that are predictive of sensitivity to IGF-1R/IR inhibitor BMS-754807 in vitro. The utility of these predictive biomarkers is different in subpopulations defined by KRAS and BRAF mutational status. FIG. 9C depicts diagrammatically what tests could be done and how results could be used for personalized treatment of CRC patients with IGF-1R/IR inhibitors. Although the clinical validity of these candidate biomarkers for predicting response to IGF-1R/IR inhibitors remains to be tested, our results provide a hypothesis that warrants testing in clinical investigation.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

The entire disclosure of each document cited (including patents, patent applications, journal articles, abstracts, laboratory manuals, books, GENBANK® Accession numbers, SWISS-PROT® Accession numbers, or other disclosures) in the Background of the Invention, Detailed Description, Brief Description of the Figures, and Examples is hereby incorporated herein by reference in their entirety. Further, the hard copy of the Sequence Listing submitted herewith, in addition to its corresponding Computer Readable Form, are incorporated herein by reference in their entireties.

1. A method for predicting the likelihood a patient will respond therapeutically to a cancer treatment comprising the administration of an IGF-1R/IR inhibitor, comprising the steps of:
(a) measuring the copy number of IRS2 in a sample from said patient, and
(b) predicting an increased likelihood said patient will respond therapeutically to said cancer treatment if said patient has an increased or elevated IRS2 copy number.

2. A method for predicting the likelihood a patient will respond therapeutically to a cancer treatment comprising the administration of an IGF-1R/IR inhibitor, comprising the steps of:
(a) measuring the copy number of IRS2 in a sample from said patient, and if said sample indicates said patent has an increased or elevated IRS2 copy number,
(b) assessing the KRAS status of said patient, and
(c) predicting an increased likelihood said patient will be sensitive to said cancer treatment if said patient has an increased or elevated IRS2 copy number in conjunction with the presence of a KRAS mutation other than a G13D mutation, or if said patient has an increased or elevated IRS2 copy number in conjunction with the presence of a wild type KRAS.

3. A method for predicting the likelihood a patient will respond therapeutically to a cancer treatment comprising the administration of an IGF-1R/IR inhibitor, comprising the steps of:
(a) measuring the copy number of IRS2 in a sample from said patient, and if said sample indicates said patent has an increased or elevated IRS2 copy number,
(b) assessing the KRAS mutation status of said patient,
(c) assessing the BRAF mutation status of said patient, and
(d) predicting an increased likelihood said patient will be sensitive to said cancer treatment if said patient has an increased or elevated IRS2 copy number in conjunction with both the presence of a KRAS mutation other than a G13D mutation and wild type BRAF, or if said patient has an increased or elevated IRS2 copy number in conjunction with the presence of a wild type KRAS and wild tune BRAF.

4. A method for predicting the likelihood a patient will respond therapeutically to a cancer treatment comprising the administration of an IGF-1R/IR inhibitor, comprising the steps of:
(a) measuring the copy number of IRS2 in a sample from said patient, and if said sample indicates said patent has a normal or decreased IRS2 copy number,
(b) assessing the KRAS status of said patient, and
(c) predicting an increased likelihood said patient will be at least partially resistant to said cancer treatment if said patient has a normal or decreased IRS2 copy number in conjunction with the presence of a KRAS mutation.

5. The method according to claim 4, wherein if said patient is KRAS wild type, further comprising the steps of:
(d) measuring the expression level of IGFBP6 in a sample from said patient, and
(e) predicting an increased likelihood said patient will respond to said cancer treatment if said sample shows said patient has a normal or decreased expression level of IGFBP6, and predicting a decreased likelihood said patient will respond to said cancer treatment if said sample shows said patient has an elevated expression level of IGFBP6.

6. A method for predicting the likelihood a patient will respond therapeutically to a cancer treatment comprising the administration of an IGF-1R/IR inhibitor, comprising the steps of:
(a) measuring the expression level of IR-A in a sample from said patient,
(b) assessing the KRAS mutation status of said patient,
(c) assessing the BRAF mutation status of said patient, and
(d) predicting an increased likelihood said patient will be sensitive to said cancer treatment if said patient has an increased or elevated IR-A expression level in conjunction with both the presence of a wild type KRAS and wild type BRAF.

7. A method for predicting the likelihood a patient will respond therapeutically to a cancer treatment comprising the administration of an IGF-1R/IR inhibitor, comprising the steps of:
(a) measuring the expression level of IGFBP6 in a sample from said patient,
(b) assessing the KRAS mutation status of said patient,
(c) assessing the BRAF mutation status of said patient, and
(d) predicting an increased likelihood said patient will be sensitive to said cancer treatment if said patient has a decreased IGFPB6 expression level in conjunction with both the presence of a wild type KRAS and wild type BRAF.

8. A method for predicting the likelihood a patient will respond therapeutically to a cancer treatment comprising the administration of an IGF-1R/IR inhibitor, comprising the steps of:
(a) measuring KRAS mutation status of said patient, and
(b) predicting an decreased likelihood said patient will respond therapeutically to said cancer treatment if said patient has a G13D KRAS mutation.

9. A method for predicting the likelihood a patient will respond therapeutically to a cancer treatment comprising the administration of an IGF-1R/IR inhibitor, comprising the steps of:
(a) measuring BRAF mutation status of said patient, and
(b) predicting an decreased likelihood said patient will respond therapeutically to said cancer treatment if said patient has a V600E BRAF mutation.

10. A method for predicting the likelihood a patient will respond therapeutically to a cancer treatment comprising the administration of an IGF-1R/IR inhibitor, comprising the steps of:
(a) measuring the expression level of IGF1R in a sample from said patient, and if said sample indicates said patent has an increased or elevated IGF1R expression level,
(b) assessing the KRAS status of said patient, and
(c) predicting an increased likelihood said patient will be sensitive to said cancer treatment if said patient has an increased or elevated IGF1R expression level in conjunction with the presence of a KRAS mutation other than a G13D mutation.

11. A method for treating a patient with cancer comprising the steps of:
(a) measuring the copy number of IRS2 in a sample from said patient, and if said sample indicates said patent has an elevated or increased IRS2 copy number,
(b) assessing the KRAS mutation status of said patient, and if said patient has either a KRAS mutation other than a G13D KRAS mutation, or is wild type KRAS,
(c) administering to said patient a therapeutically acceptable amount of an IGF-1R/IR inhibitor.

12. A method for treating a patient with cancer comprising the steps of:
(a) measuring the copy number of IRS2 in a sample from said patient, and if said sample indicates said patent has a normal or decreased IRS2 copy number,
(b) assessing the KRAS status of said patient, and if said patient has a normal or decreased IRS2 copy number in conjunction with the presence of a wild type KRAS, further comprising the steps of
(c) measuring the expression level of IGFBP6, and if said sample shows said patient has a normal or decreased expression level of IGFBP6,
(d) administering to said patient a therapeutically acceptable amount of an IGF-1R/IR inhibitor.

13. A method for treating a patient with cancer comprising the steps of:
(a) measuring the expression level of IGFBP6, wherein if said sample shows said patient has an reduce or decreased expression level of IGFBP6,
(b) assessing the KRAS mutation status and BRAF mutation status of said patient, and if said patient has a decreased IGFBP6 expression level in conjunction with the presence of a wild type KRAS and wild type BRAF,
(c) administering to said patient a therapeutically acceptable amount of an IGF-1R inhibitor.

14. A method for treating a patient with cancer comprising the steps of:
(a) measuring the expression level of IGF1R, wherein if said sample shows said patient has an increased or elevated expression level of IGF1R,
(b) assessing the KRAS mutation status and BRAF mutation status of said patient, and if said patient has an increased IGF IR expression level in conjunction with the presence of a wild type KRAS and wild type BRAF,
(c) administering to said patient a therapeutically acceptable amount of an IGF-1R inhibitor.

15. A method for treating a patient with cancer comprising the steps of:
(a) measuring the expression level of IR-A, wherein if said sample shows said patient has an increased or elevated expression level of IR-A,
(b) assessing the KRAS mutation status and BRAF mutation status of said patient, and if said patient has an increased IR-A expression level in conjunction with the presence of a wild type KRAS and wild type BRAF,
(c) administering to said patient a therapeutically acceptable amount of an IGF-1R inhibitor.

16-20. (canceled)

21. A kit for use in treating a patient with cancer, comprising:
(a) a means for measuring the IRS2 copy number in a patient sample;
(b) a therapeutically effective amount of an IGF-1R/IR inhibitor, and instructions for administering said IGF-1R/IR inhibitor if said patient has an increased or elevated IRS2 copy number.

22. A kit for use in treating a patient with cancer, comprising:
(a) a means for measuring the IRS2 copy number in a patient sample;
(b) a means for determining the KRAS mutation status of said patient sample or a means for determining the BRAF mutation status;
(c) a therapeutically effective amount of an IGF-1R inhibitor, and instructions for administering said IGF-1R inhibitor if said patient has wild type KRAS or KRAS mutation other than a G13D mutation, a wild type BRAF, and has an increased or elevated IRS2 copy number.

23. A kit for use in treating a patient with cancer, comprising:
(a) a means for measuring the BRAF mutation status in a patient sample;
(b) a means for determining the KRAS mutation status of said patient sample;
(c) a means for measuring the IGFBP6, IR-A, or IGF1R expression level in a patient sample, and
(d) a therapeutically effective amount of an IGF-1R/IR inhibitor, and instructions for administering said IGF-1R/IR inhibitor if said patient is KRAS wild type, is BRAF wild type, and has either a decreased IGFBP6 expression level, or an increased IGF1R or IR-A level.

24. A kit for use in treating a patient with cancer, comprising:
(a) a means for measuring the IGFBP6 expression level in a patient sample,
(b) a means for determining the KRAS mutation status of said patient sample; and
(c) a therapeutically effective amount of an IGF-1R inhibitor, and instructions for administering said IGF-1R inhibitor if said patient is KRAS wild type, and has a decreased IGFBP6 expression level.


SEQUENCE LISTING (11 sequences)

SEQ ID NO: 1 - DNA, 4017 bp, homo sapiens
atggcgagcccgccgcggcacgggccgcccgggccggcgagcggagacggccccaacctcaacaacaacaacaacaacaacaaccacagcgtgcgcaagtgcggctacctgcgcaagcagaagcatggccacaagcgcttcttcgtgctgcgcggacccggcgcgggcggcgacgaggcgacggcgggcggggggtcggcgccgcaaccgccgcggctcgagtactacgagagcgagaaaaagtggcggagcaaggcaggcgcgccgaaacgggtgatcgctctcgactgctgcctgaacatcaacaagcgcgccgacgccaagcacaagtacctgatcgccctctacaccaaggacgagtacttcgccgtggccgccgagaacgagcaggagcaggagggctggtaccgcgcgctcaccgacctggtcagcgagggccgcgcggccgccggagacgcgccccccgccgccgcgcccgccgcgtcctgcagcgcctccctgcccggcgccctgggcggctctgccggcgccgccggggccgaggacagctacgggctggtggctcccgccacggccgcctaccgtgaggtgtggcaggtgaacctgaagcccaagggtctgggccagagcaagaacctgacgggggtgtaccgtctgtgcctgtctgcgcgcaccatcggcttcgtgaagctcaactgcgagcagccgtcggtgacgctgcagctcatgaacatccgccgctgcggccactcggacagcttcttcttcatcgaggtgggccgctcggccgtcacaggccccggcgagctgtggatgcaggcggacgactcggtggtggcgcagaacatccacgagaccatcctggaggccatgaaggcgctcaaggagctcttcgagttccggccgcgcagtaagagccaatcgtcggggtcgtcggccacgcaccccatcagcgtccccggcgcgcgccgccaccaccacctggtcaacctgccccccagccagacgggcctggtgcgccgctcgcgcaccgacagcctggccgccaccccgccggcggccaagtgcagctcgtgccgggtgcgcaccgccagcgagggcgacggcggcgcggcggcgggagcggcggccgcgggcgccaggccggtgtcggtggctgggagccccctgagccccgggccggtgcgcgcgcccctgagccgctcgcacaccctgagcggcggctgcggcggccgcgggagcaaggtggcgctgctgccggcagggggcgcgctgcaacacagccgctccatgtccatgcccgtggcgcactcgccgcccgccgccaccagccccggctccctgtcgtccagcagcggccacggctcgggctcctacccgccgccgcccggcccgcacccgcctctgccgcatccgctgcaccacggccccggccagcggccctccagcggcagcgcctccgcctcgggctcccccagcgaccccggcttcatgtccctggacgagtacggctccagcccaggcgacctgcgcgccttctgcagccaccgaagcaacacgcccgagtccatcgcggagacgcccccggcccgagacggcggcggcggcggtgagttctacgggtacatgaccatggacaggcccctgagccactgtggccgctcctaccgccgggtctcgggggacgcggcccaggacctggaccgagggctgcgcaagaggacctactccctgaccacgccagcccggcagcggccggtgccccagccctcctctgcctcgctggatgaatacaccctgatgcgggccaccttctcgggcagcgcgggccgcctctgcccgtcctgccccgcgtcctctcccaaggtggcctaccacccctacccagaggactacggagacatcgagatcggctcccacaggagctccagcagcaacctgggggcagacgacggctacatgcccatgacgcccggcgcggccctcgcgggcagtgggagcggcagctgcaggagcgacgactacatgcccatgagccccgccagcgtgtccgcccccaagcagatcttgcagcccagggccgccgccgccgccgccgccgccgtgccttctgcggggcctgcggggccagcacccacctctgcggcgggcaggacattcccggcgagcgggggcggctacaaggccagctcgcccgccgagagctcccccgaggacagtgggtacatgcgcatgtggtgcggttccaagctgtccatggagcatgcagatggcaagctgctgcccaacggggactacctcaacgtgtcccccagcgacgcggtcaccacgggcaccccgcccgacttcttctccgcagccctgcaccccggcggggagccgctcaggggcgttcccggctgctgctacagctccttgccccgctcctacaaggccccctacacctgtggcggggacagcgaccagtacgtgctcatgagctcccccgtggggcgcatcctggaggaggagcgtctggagcctcaggccacgccagggcccagccaggcggccagcgccttcggggccggccccacgcagccccctcaccctgtagtgccttcgcccgtgcggcctagcggcggccgcccggagggcttcttgggccagcgcggccgggcggtgaggcccacgcgcctgtccctggaggggctgcccagcctgcccagcatgcacgagtacccactgccaccggagcccaagagccccggcgagtacatcaacatcgactttggcgagcccggggcccgcctgtcgccgcccgcgcctcccctgctggcgtcggcggcctcgtcctcctcgctcttgtccgccagcagcccggcctcgtcgctgggctcaggcaccccgggcaccagcagcgacagccggcagcggtctccgctctccgactacatgaacctcgacttcagctcccccaagtctcctaagccgggcgccccgagcggccaccccgtgggctccttggacggcctcctgtcccccgaggcctcctccccgtatccgccgttgcccccgcgtccgtccgcgtccccgtcgtcgtctctgcagccgccgccaccgccgccggccccgggggagctgtaccgcctgccccccgcctcggccgttgccaccgcccagggcccgggcgccgcctcatcgttgtcctcggacaccggggacaatggtgactacaccgagatggcttttggtgtggccgccaccccgccgcaacctatcgcggcccccccgaagccagaagctgcccgcgtggccagcccgacgtcgggcgtgaagaggctgagcctcatggagcaggtgtcgggagtcgaggccttcctgcaggccagccagcccccggacccccaccgcggcgccaaggtcatccgcgcagacccgcaggggggccgccgccgccacagttccgagaccttctcctccaccacgacggtcacccccgtgtccccgtccttcgcccacaaccccaagcgccacaactcggcctccgtggaaaatgtctctctcaggaaaagcagcgagggcggcgtgggtgtcggccctggagggggcgacgagccgcccacctccccacgacagttgcagccggcgccccctttggcaccgcagggccggccgtggaccccgggtcagcccgggggcttggtcggttgtcctgggagcggtggatcgcccatgcgcagagagacctctgccggcttccagaatggtctcaactacatcgccatcgacgtgagggaggagcccgggctgccaccccagccgcagccgccgccgccgccgcttcctcagccgggagacaagagctcctggggccggacccgaagcctcgggggtctcatcagcgctgtgggcgtcggcagcaccggcggcgggtgcggggggccgggtcccggtgccctgccccctgccaacacctacgccagcattgacttcttgtcccaccacttgaaggaggccaccatcgtgaaagagtga

SEQ ID NO: 2 - protein, 1338 aa, homo sapiens
MASPPRHGPPGPASGDGPNLNNNNNNNNHSVRKCGYLRKQKHGHKRFFVLRGPGAGGDEATAGGGSAPQPPRLEYYESEKKWRSKAGAPKRVIALDCCLNINKRADAKHKYLIALYTKDEYFAVAAENEQEQEGWYRALTDLVSEGRAAAGDAPPAAAPAASCSASLPGALGGSAGAAGAEDSYGLVAPATAAYREVWQVNLKPKGLGQSKNLTGVYRLCLSARTIGFVKLNCEQPSVTLQLMNIRRCGHSDSFFFIEVGRSAVTGPGELWMQADDSVVAQNIHETILEAMKALKELFEFRPRSKSQSSGSSATHPISVPGARRHHHLVNLPPSQTGLVRRSRTDSLAATPPAAKCSSCRVRTASEGDGGAAAGAAAAGARPVSVAGSPLSPGPVRAPLSRSHTLSGGCGGRGSKVALLPAGGALQHSRSMSMPVAHSPPAATSPGSLSSSSGHGSGSYPPPPGPHPPLPHPLHHGPGQRPSSGSASASGSPSDPGFMSLDEYGSSPGDLRAFCSHRSNTPESIAETPPARDGGGGGEFYGYMTMDRPLSHCGRSYRRVSGDAAQDLDRGLRKRTYSLTTPARQRPVPQPSSASLDEYTLMRATFSGSAGRLCPSCPASSPKVAYHPYPEDYGDIEIGSHRSSSSNLGADDGYMPMTPGAALAGSGSGSCRSDDYMPMSPASVSAPKQILQPRAAAAAAAAVPSAGPAGPAPTSAAGRTFPASGGGYKASSPAESSPEDSGYMRMWCGSKLSMEHADGKLLPNGDYLNVSPSDAVTTGTPPDFFSAALHPGGEPLRGVPGCCYSSLPRSYKAPYTCGGDSDQYVLMSSPVGRILEEERLEPQATPGPSQAASAFGAGPTQPPHPVVPSPVRPSGGRPEGFLGQRGRAVRPTRLSLEGLPSLPSMHEYPLPPEPKSPGEYINIDFGEPGARLSPPAPPLLASAASSSSLLSASSPASSLGSGTPGTSSDSRQRSPLSDYMNLDFSSPKSPKPGAPSGHPVGSLDGLLSPEASSPYPPLPPRPSASPSSSLQPPPPPPAPGELYRLPPASAVATAQGPGAASSLSSDTGDNGDYTEMAFGVAATPPQPIAAPPKPEAARVASPTSGVKRLSLMEQVSGVEAFLQASQPPDPHRGAKVIRADPQGGRRRHSSETFSSTTTVTPVSPSFAHNPKRHNSASVENVSLRKSSEGGVGVGPGGGDEPPTSPRQLQPAPPLAPQGRPWTPGQPGGLVGCPGSGGSPMRRETSAGFQNGLNYIAIDVREEPGLPPQPQPPPPPLPQPGDKSSWGRTRSLGGLISAVGVGSTGGGCGGPGPGALPPANTYASIDFLSHHLKEATIVKE

SEQ ID NO: 3 - DNA, 723 bp, homo sapiens
atgaccccccacaggctgctgccaccgctgctgctgctgctagctctgctgctcgctgccagcccaggaggcgccttggcgcggtgcccaggctgcgggcaaggggtgcaggcgggttgtccagggggctgcgtggaggaggaggatggggggtcgccagccgagggctgcgcggaagctgagggctgtctcaggagggaggggcaggagtgcggggtctacacccctaactgcgccccaggactgcagtgccatccgcccaaggacgacgaggcgcctttgcgggcgctgctgctcggccgaggccgctgccttccggcccgcgcgcctgctgttgcagaggagaatcctaaggagagtaaaccccaagcaggcactgcccgcccacaggatgtgaaccgcagagaccaacagaggaatccaggcacctctaccacgccctcccagcccaattctgcgggtgtccaagacactgagatgggcccatgccgtagacatctggactcagtgctgcagcaactccagactgaggtctaccgaggggctcaaacactctacgtgcccaattgtgaccatcgaggcttctaccggaagcggcagtgccgctcctcccaggggcagcgccgaggtccctgctggtgtgtggatcggatgggcaagtccctgccagggtctccagatggcaatggaagctcctcctgccccactgggagtagcggctaa

SEQ ID NO: 4 - protein, 240 aa, homo sapiens
MTPHRLLPPLLLLLALLLAASPGGALARCPGCGQGVQAGCPGGCVEEEDGGSPAEGCAEAEGCLRREGQECGVYTPNCAPGLQCHPPKDDEAPLRALLLGRGRCLPARAPAVAEENPKESKPQAGTARPQDVNRRDQQRNPGTSTTPSQPNSAGVQDTEMGPCRRHLDSVLQQLQTEVYRGAQTLYVPNCDHRGFYRKRQCRSSQGQRRGPCWCVDRMGKSLPGSPDGNGSSSCPTGSSG

SEQ ID NO: 5 - DNA, 4149 bp, homo sapiens
atggccaccgggggccggcggggggcggcggccgcgccgctgctggtggcggtggccgcgctgctactgggcgccgcgggccacctgtaccccggagaggtgtgtcccggcatggatatccggaacaacctcactaggttgcatgagctggagaattgctctgtcatcgaaggacacttgcagatactcttgatgttcaaaacgaggcccgaagatttccgagacctcagtttccccaaactcatcatgatcactgattacttgctgctcttccgggtctatgggctcgagagcctgaaggacctgttccccaacctcacggtcatccggggatcacgactgttctttaactacgcgctggtcatcttcgagatggttcacctcaaggaactcggcctctacaacctgatgaacatcacccggggttctgtccgcatcgagaagaacaatgagctctgttacttggccactatcgactggtcccgtatcctggattccgtggaggataattacatcgtgttgaacaaagatgacaacgaggagtgtggagacatctgtccgggtaccgcgaagggcaagaccaactgccccgccaccgtcatcaacgggcagtttgtcgaacgatgttggactcatagtcactgccagaaagtttgcccgaccatctgtaagtcacacggctgcaccgccgaaggcctctgttgccacagcgagtgcctgggcaactgttctcagcccgacgaccccaccaagtgcgtggcctgccgcaacttctacctggacggcaggtgtgtggagacctgcccgcccccgtactaccacttccaggactggcgctgtgtgaacttcagcttctgccaggacctgcaccacaaatgcaagaactcgcggaggcagggctgccaccagtacgtcattcacaacaacaagtgcatccctgagtgtccctccgggtacacgatgaattccagcaacttgctgtgcaccccatgcctgggtccctgtcccaaggtgtgccacctcctagaaggcgagaagaccatcgactcggtgacgtctgcccaggagctccgaggatgcaccgtcatcaacgggagtctgatcatcaacattcgaggaggcaacaatctggcagctgagctagaagccaacctcggcctcattgaagaaatttcagggtatctaaaaatccgccgatcctacgctctggtgtcactttccttcttccggaagttacgtctgattcgaggagagaccttggaaattgggaactactccttctatgccttggacaaccagaacctaaggcagctctgggactggagcaaacacaacctcaccatcactcaggggaaactcttcttccactataaccccaaactctgcttgtcagaaatccacaagatggaagaagtttcaggaaccaaggggcgccaggagagaaacgacattgccctgaagaccaatggggaccaggcatcctgtgaaaatgagttacttaaattttcttacattcggacatcttttgacaagatcttgctgagatgggagccgtactggccccccgacttccgagacctcttggggttcatgctgttctacaaagaggccccttatcagaatgtgacggagttcgacgggcaggatgcgtgtggttccaacagttggacggtggtagacattgacccacccctgaggtccaacgaccccaaatcacagaaccacccagggtggctgatgcggggtctcaagccctggacccagtatgccatctttgtgaagaccctggtcaccttttcggatgaacgccggacctatggggccaagagtgacatcatttatgtccagacagatgccaccaacccctctgtgcccctggatccaatctcagtgtctaactcatcatcccagattattctgaagtggaaaccaccctccgaccccaatggcaacatcacccactacctggttttctgggagaggcaggcggaagacagtgagctgttcgagctggattattgcctcaaagggctgaagctgccctcgaggacctggtctccaccattcgagtctgaagattctcagaagcacaaccagagtgagtatgaggattcggccggcgaatgctgctcctgtccaaagacagactctcagatcctgaaggagctggaggagtcctcgtttaggaagacgtttgaggattacctgcacaacgtggttttcgtccccagaaaaacctcttcaggcactggtgccgaggaccctaggccatctcggaaacgcaggtcccttggcgatgttgggaatgtgacggtggccgtgcccacggtggcagctttccccaacacttcctcgaccagcgtgcccacgagtccggaggagcacaggccttttgagaaggtggtgaacaaggagtcgctggtcatctccggcttgcgacacttcacgggctatcgcatcgagctgcaggcttgcaaccaggacacccctgaggaacggtgcagtgtggcagcctacgtcagtgcgaggaccatgcctgaagccaaggctgatgacattgttggccctgtgacgcatgaaatctttgagaacaacgtcgtccacttgatgtggcaggagccgaaggagcccaatggtctgatcgtgctgtatgaagtgagttatcggcgatatggtgatgaggagctgcatctctgcgtctcccgcaagcacttcgctctggaacggggctgcaggctgcgtgggctgtcaccggggaactacagcgtgcgaatccgggccacctcccttgcgggcaacggctcttggacggaacccacctatttctacgtgacagactatttagacgtcccgtcaaatattgcaaaaattatcatcggccccctcatctttgtctttctcttcagtgttgtgattggaagtatttatctattcctgagaaagaggcagccagatgggccgctgggaccgctttacgcttcttcaaaccctgagtatctcagtgccagtgatgtgtttccatgctctgtgtacgtgccggacgagtgggaggtgtctcgagagaagatcaccctccttcgagagctggggcagggctccttcggcatggtgtatgagggcaatgccagggacatcatcaagggtgaggcagagacccgcgtggcggtgaagacggtcaacgagtcagccagtctccgagagcggattgagttcctcaatgaggcctcggtcatgaagggcttcacctgccatcacgtggtgcgcctcctgggagtggtgtccaagggccagcccacgctggtggtgatggagctgatggctcacggagacctgaagagctacctccgttctctgcggccagaggctgagaataatcctggccgccctccccctacccttcaagagatgattcagatggcggcagagattgctgacgggatggcctacctgaacgccaagaagtttgtgcatcgggacctggcagcgagaaactgcatggtcgcccatgattttactgtcaaaattggagactttggaatgaccagagacatctatgaaacggattactaccggaaagggggcaagggtctgctccctgtacggtggatggcaccggagtccctgaaggatggggtcttcaccacttcttctgacatgtggtcctttggcgtggtcctttgggaaatcaccagcttggcagaacagccttaccaaggcctgtctaatgaacaggtgttgaaatttgtcatggatggagggtatctggatcaacccgacaactgtccagagagagtcactgacctcatgcgcatgtgctggcaattcaaccccaagatgaggccaaccttcctggagattgtcaacctgctcaaggacgacctgcaccccagctttccagaggtgtcgttcttccacagcgaggagaacaaggctcccgagagtgaggagctggagatggagtttgaggacatggagaatgtgcccctggaccgttcctcgcactgtcagagggaggaggcggggggccgggatggagggtcctcgctgggtttcaagcggagctacgaggaacacatcccttacacacacatgaacggaggcaagaaaaacgggcggattctgaccttgcctcggtccaatccttcctaa

SEQ ID NO: 6 - protein, 1382 aa, homo sapiens
MATGGRRGAAAAPLLVAVAALLLGAAGHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRKTSSGTGAEDPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAKIIIGPLIFVFLFSVVIGSIYLFLRKRQPDGPLGPLYASSNPEYLSASDVFPCSVYVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIYETDYYRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENKAPESEELEMEFEDMENVPLDRSSHCQREEAGGRDGGSSLGFKRSYEEHIPYTHMNGGKKNGRILTLPRSNPS

SEQ ID NO: 7 - DNA, 4104 bp, homo sapiens
atgaagtctggctccggaggagggtccccgacctcgctgtgggggctcctgtttctctccgccgcgctctcgctctggccgacgagtggagaaatctgcgggccaggcatcgacatccgcaacgactatcagcagctgaagcgcctggagaactgcacggtgatcgagggctacctccacatcctgctcatctccaaggccgaggactaccgcagctaccgcttccccaagctcacggtcattaccgagtacttgctgctgttccgagtggctggcctcgagagcctcggagacctcttccccaacctcacggtcatccgcggctggaaactcttctacaactacgccctggtcatcttcgagatgaccaatctcaaggatattgggctttacaacctgaggaacattactcggggggccatcaggattgagaaaaatgctgacctctgttacctctccactgtggactggtccctgatcctggatgcggtgtccaataactacattgtggggaataagcccccaaaggaatgtggggacctgtgtccagggaccatggaggagaagccgatgtgtgagaagaccaccatcaacaatgagtacaactaccgctgctggaccacaaaccgctgccagaaaatgtgcccaagcacgtgtgggaagcgggcgtgcaccgagaacaatgagtgctgccaccccgagtgcctgggcagctgcagcgcgcctgacaacgacacggcctgtgtagcttgccgccactactactatgccggtgtctgtgtgcctgcctgcccgcccaacacctacaggtttgagggctggcgctgtgtggaccgtgacttctgcgccaacatcctcagcgccgagagcagcgactccgaggggtttgtgatccacgacggcgagtgcatgcaggagtgcccctcgggcttcatccgcaacggcagccagagcatgtactgcatcccttgtgaaggtccttgcccgaaggtctgtgaggaagaaaagaaaacaaagaccattgattctgttacttctgctcagatgctccaaggatgcaccatcttcaagggcaatttgctcattaacatccgacgggggaataacattgcttcagagctggagaacttcatggggctcatcgaggtggtgacgggctacgtgaagatccgccattctcatgccttggtctccttgtccttcctaaaaaaccttcgcctcatcctaggagaggagcagctagaagggaattactccttctacgtcctcgacaaccagaacttgcagcaactgtgggactgggaccaccgcaacctgaccatcaaagcagggaaaatgtactttgctttcaatcccaaattatgtgtttccgaaatttaccgcatggaggaagtgacggggactaaagggcgccaaagcaaaggggacataaacaccaggaacaacggggagagagcctcctgtgaaagtgacgtcctgcatttcacctccaccaccacgtcgaagaatcgcatcatcataacctggcaccggtaccggccccctgactacagggatctcatcagcttcaccgtttactacaaggaagcaccctttaagaatgtcacagagtatgatgggcaggatgcctgcggctccaacagctggaacatggtggacgtggacctcccgcccaacaaggacgtggagcccggcatcttactacatgggctgaagccctggactcagtacgccgtttacgtcaaggctgtgaccctcaccatggtggagaacgaccatatccgtggggccaagagtgagatcttgtacattcgcaccaatgcttcagttccttccattcccttggacgttctttcagcatcgaactcctcttctcagttaatcgtgaagtggaaccctccctctctgcccaacggcaacctgagttactacattgtgcgctggcagcggcagcctcaggacggctacctttaccggcacaattactgctccaaagacaaaatccccatcaggaagtatgccgacggcaccatcgacattgaggaggtcacagagaaccccaagactgaggtgtgtggtggggagaaagggccttgctgcgcctgccccaaaactgaagccgagaagcaggccgagaaggaggaggctgaataccgcaaagtctttgagaatttcctgcacaactccatcttcgtgcccagacctgaaaggaagcggagagatgtcatgcaagtggccaacaccaccatgtccagccgaagcaggaacaccacggccgcagacacctacaacatcaccgacccggaagagctggagacagagtaccctttctttgagagcagagtggataacaaggagagaactgtcatttctaaccttcggcctttcacattgtaccgcatcgatatccacagctgcaaccacgaggctgagaagctgggctgcagcgcctccaacttcgtctttgcaaggactatgcccgcagaaggagcagatgacattcctgggccagtgacctgggagccaaggcctgaaaactccatctttttaaagtggccggaacctgagaatcccaatggattgattctaatgtatgaaataaaatacggatcacaagttgaggatcagcgagaatgtgtgtccagacaggaatacaggaagtatggaggggccaagctaaaccggctaaacccggggaactacacagcccggattcaggccacatctctctctgggaatgggtcgtggacagatcctgtgttcttctatgtccaggccaaaacaggatatgaaaacttcatccatctgatcatcgctctgcccgtcgctgtcctgttgatcgtgggagggttggtgattatgctgtacgtcttccatagaaagagaaataacagcaggctggggaatggagtgctgtatgcctctgtgaacccggagtacttcagcgctgctgatgtgtacgttcctgatgagtgggaggtggctcgggagaagatcaccatgagccgggaacttgggcaggggtcgtttgggatggtctatgaaggagttgccaagggtgtggtgaaagatgaacctgaaaccagagtggccattaaaacagtgaacgaggccgcaagcatgcgtgagaggattgagtttctcaacgaagcttctgtgatgaaggagttcaattgtcaccatgtggtgcgattgctgggtgtggtgtcccaaggccagccaacactggtcatcatggaactgatgacacggggcgatctcaaaagttatctccggtctctgaggccagaaatggagaataatccagtcctagcacctccaagcctgagcaagatgattcagatggccggagagattgcagacggcatggcatacctcaacgccaataagttcgtccacagagaccttgctgcccggaattgcatggtagccgaagatttcacagtcaaaatcggagattttggtatgacgcgagatatctatgagacagactattaccggaaaggagggaaagggctgctgcccgtgcgctggatgtctcctgagtccctcaaggatggagtcttcaccacttactcggacgtctggtccttcggggtcgtcctctgggagatcgccacactggccgagcagccctaccagggcttgtccaacgagcaagtccttcgcttcgtcatggagggcggccttctggacaagccagacaactgtcctgacatgctgtttgaactgatgcgcatgtgctggcagtataaccccaagatgaggccttccttcctggagatcatcagcagcatcaaagaggagatggagcctggcttccgggaggtctccttctactacagcgaggagaacaagctgcccgagccggaggagctggacctggagccagagaacatggagagcgtccccctggacccctcggcctcctcgtcctccctgccactgcccgacagacactcaggacacaaggccgagaacggccccggccctggggtgctggtcctccgcgccagcttcgacgagagacagccttacgcccacatgaacgggggccgcaagaacgagcgggccttgccgctgccccagtcttcgacctgctga

SEQ ID NO: 8 - protein, 1367 aa, homo sapiens
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 9 - DNA, 18 bp, Artificial
  Forward oligonucleotide primer
tttcgtccccaggccatc

SEQ ID NO: 10 - DNA, 16 bp, Artificial
  Reverse oligonucleotide primer
gcccgtgaagtgtcgc

SEQ ID NO: 11 - DNA, 18 bp, Artificial
  Oligonucleotide probe
ttgagaaggtggtgaaca